(19) United States
(12) Patent Application Publication
(10) Pub. No.: US 2026/0224716 A1
(43) Pub. Date: Aug. 6, 2026

(54) PTH ANALOGS FOR THE TREATMENT OF HYPOPARATHYROIDISM

(71) Applicant: INDIANA UNIVERSITY RESEARCH AND TECHNOLOGY CORPORATION, Indianapolis, IN (US)

(72) Inventors: Richard D. DIMARCHI, Carmel, IN (US); Fa ZHANG, Bloomington, IN (US)

(21) Appl. No.: 19/413,817

(22) Filed: Dec. 9, 2025

Related U.S. Application Data
(63) Continuation of application No. 17/925,477, filed on May 25, 2021.
(60) Provisional application No. 63/030,004, filed on May 26, 2020.
(60) Provisional application No. 63/033,586, filed on Jun. 2, 2020.

(51) Int. Cl. A61K 47/64; A61K 9/20; A61P 5/18; A61P 19/10; C07K 14/635

(52) U.S. Cl. CPC A61K 47/64; A61K 9/20; A61P 5/18; A61P 19/10; C07K 14/635

(57) ABSTRACT

A novel derivative of parathyroid hormone is provided that has an extended time of action relative to known native parathyroid hormone agonist peptides, while minimizing excessive action shortly after administration. Compositions comprising the novel parathyroid hormone conjugates can be used to treat hypoparathyroidism and osteoporosis.

### CROSS REFERENCE TO RELATED APPLICATIONS

This application claims priority to the following: U.S. Provisional Patent Application No. 63/030,004 filed on May 26, 2020 and U.S. Provisional Patent Application No. 63/033,586 filed on Jun. 2, 2020, the disclosures of which are expressly incorporated herein.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: a 230,000 bytes xml file named 433382A.xml created on May 11, 2026.

### BACKGROUND

Through secretion of parathyroid hormone (PTH), the parathyroid gland controls calcium in the blood in a very tight range (8-10 mg/dL in adults). Calcium, which is stored in bones, is released in response to PTH, supporting central nervous system functioning, blood vessel and muscle contraction, enzyme and hormone secretion, and blood clotting. Mechanistically, PTH increases concentrations of calcium by stimulating the production of biologically-active forms of vitamin D within the kidney, while mobilizing calcium and phosphate to be released from the bones, signaling kidneys to eliminate excess phosphate, and maximizing tubular reabsorption of calcium within the kidneys.

Hypoparathyroidism (hypo PT) is an uncommon disease in which there is insufficient production of PTH. Patients with hypo PT excrete too much calcium in their urine, have too much phosphate in their blood, and have abnormally low bone turnover. Until the approval of NPS/Shire's Natpara® (rhPTH) by the Food and Drug Administration (FDA) in January of 2015, hypoparathyroidism was one of the very few classic endocrine diseases not treated by hormone replacement. Natpara® provides only modest efficacy relative to the standard of care consisting of high dose calcium and vitamin D supplements. Even so, the drug has been well received since its 2015 commercial launch, demonstrating the significant unmet need that exists in treating hypo PT.

Because hypo PT is a rare disease, drugs being developed for hypo PT benefit from the Orphan Drug Designations from the FDA and European Medicines Agency (EMA). Each emerging competitor is directed at restoration of PTH in a more physiologic manner than Natpara®, to primarily normalize serum calcium and diminish long-term consequences of disease insufficiently managed. The competitors differ in their approach to normalizing PTH, with sizable differences in product profile, that include routes of administration, potency, dose frequency and therapeutic index. The compositions disclosed herein provide a weekly treatment for patients with hypo PT that will restore and maintain physiologic levels of PTH safely throughout the week, offering unique pharmacological benefit and significant convenience for patients.

Hypo PT can be divided into primary and secondary disease. Primary disease occurs when there are intrinsic defects within the parathyroid glands, from genetic causes. Hypo PT from genetic causes is especially rare, thought to cause less than 10% of total cases. Secondary, or acquired, disease occurs when a previously functioning parathyroid gland function is impaired, destroyed or ablated. Secondary disease is by far the most common, causing approximately 90% of total cases.

At least 75% of acquired hypo PT cases are caused by anterior neck surgery (i.e., total thyroidectomy or radical neck dissection for head and neck malignancies), due to inadvertent or unavoidable removal of or damage to parathyroid glands and/or their blood supply. Transient hypo PT after thyroid surgery is relatively common, occurring in an estimated 7-46% of thyroid surgeries. Transient hypo PT resolves within weeks or months of surgery. Chronic hypo PT is rare, occurring at a rate of 0.9-1.6% in surgical centers with experienced endocrine surgeons and a high case volume. However, rates as high as 6.6% have been reported post thyroid surgery, emphasizing the importance of expertise and experience in avoiding permanent damage resulting in hypo PT.

After anterior neck surgery, the next most common acquired cause of hypo PT in adults is thought to be autoimmune disease. It can affect either the parathyroid glands alone or multiple endocrine glands. Autoimmune mediated disease is estimated to cause less than 10% of acquired hypo PT. Other secondary causes include rare infiltrative disorders due to metastatic disease or iron/copper overload, ionizing radiation exposure, or can be of unknown origin (idiopathic).

Estimates of hypo PT prevalence in the US range from 60,000 to 115,000 patients. Experts suggest the best estimate, which is based on a large health plan claims database, is 77,000 patients, of which 58,793 were insured. Data for other regions are extremely limited, and documented estimates range from 70,000 to 267,000 for Europe, 20,000 for Japan, and 30,000 for the rest of the world.

Chronic hypo PT is a growing disease, driven by incidence of thyroid disease, including cancer, and its treatments, most notably surgery. For example, between 1996 and 2006, the total number of thyroidectomies performed in the US increased 39%, from 66,864 to 92,931. It is estimated that the number of thyroidectomies performed in the US each year has now reached 150,000. The number of people potentially affected by hypo PT is growing. Because thyroid disease impacts women more than men, more than 70% of patients with hypo PT are women.

Most people with calcium levels residing outside normal physiological range feel sick. Because of calcium's crucial role in nerve and muscle function, patients with hypocalcemia may experience tingling or burning in extremities (paresthesia), muscle twitching, muscle pain, involuntary muscle contractions (tetany), dry/rough skin, inability to concentrate or focus, anxiety and/or depression. Severely low levels of blood calcium can cause life-threatening laryngospasm, seizures, or cardiac arrhythmias, requiring emergency treatment with IV calcium. Serious long-term consequences of hypo PT can include nephrocalcinosis, impaired renal function/chronic kidney disease, calcium deposits in soft tissues, and overly mineralized bones.

Hypo PT is typically diagnosed through clinical history and laboratory tests. The diagnosis is typically characterized by low/undetectable levels of serum PTH and hypocalcemia, defined as total serum calcium below the lower limit of normal, and hyperphosphatemia. Levels of activated vitamin D and bone turnover markers are also typically in the lower portion of normal to overtly low range, and excretion of calcium is increased. Such lab results in the context of a recent neck surgery can result in a straightforward diagnosis of hypo PT. However, cases of hypo PT can be very difficult to diagnose, especially when there is no known damage to the parathyroid gland.

There are no definitive guidelines for the treatment of hypo PT, so treatment is based upon experience and clinical judgment. Generally accepted primary goals of chronic treatment are to maintain within an acceptable range serum total calcium (low to low-normal range), serum phosphorus (high-normal range), 24-hour urine calcium excretion (<7.5 mmol/d) and calcium phosphate product (under 4.4 mmol₂/L₂).

Standard treatment includes calcium, vitamin D metabolites, and sometimes thiazide diuretics. Recommended calcium supplements are calcium carbonate and calcium citrate, and the amount needed varies greatly (9-fold) among patients. 1,25(OH)₂D₃ (calcitriol) is an active metabolite of vitamin D and helps maintain serum calcium by improving the efficiency of intestinal calcium absorption. Calcitriol is also administered over a wide (8-fold) dosing range. By enhancing distal renal tubular calcium reabsorption, thiazide diuretics (drugs in the benzothiadiazines class) can also be useful in treating hypo PT.

While it may sound straightforward to replenish deficient calcium with calcium and vitamin D supplements, the reality is that it is extremely difficult to achieve good control. Most patients experience a roller coaster in dosing levels that alternate between too high and too low. Also, calcium and vitamin D supplements do nothing to restore the underlying PTH deficiency, which has therapeutic consequence.

Patients with hypo PT experience a heavy burden of illness, with significant, negative impact to their daily quality of life, and to the lives of their caregivers, families, and friends. NPS Pharmaceuticals conducted a 374-patient epidemiology study, PARADOX, to assess clinical, social, and economic implications of hypo PT. Data were collected through a 30-minute, web-based instrument which was developed with input from clinical experts, the Hypoparathyroidism Association, and patients. This instrument was primarily disseminated via email to Hypoparathyroidism Association members, including adults in the US who had been living with hypo PT for six or more months. Results were published by Hadker et al in Endocrine Practice, with the following key highlights:
72% experienced more than 10 symptoms daily, with the most frequently reported:
   Physical symptoms: fatigue (82%), muscle pain/cramping (78%), paresthesia (76%), tetany (70%), joint or bone pain (67%), and pain or weakness in the extremities (53%)
   Emotional symptoms: anxiety (59%) and depression (53%)
   Cognitive symptoms: brain fog/mental lethargy (72%), inability to concentrate (65%), memory loss (61.5%) and sleep disturbances (57%)
   79% required hospital stays or emergency department visits
   45% reported significant interference with their lives
   85% reported an inability to perform household activities, and
   20% experienced a disease-associated (negative) change in employment status.

Other publications have also confirmed the illness burden of hypo PT. A cross-sectional study published by Arlt et al in the European Journal of Endocrinology compared well-being and mood, using validated questionnaires in 25 women with postsurgical hypo PT who were stably managed with treatment of calcium and vitamin D to 25 women with intact parathyroid function following thyroid surgery. Hypo PT patients had significantly higher global complaint scores in the Geissen complaint list, the von Zerssen symptom list, and Symptom Checklist-90, with increases in subscale scores for anxiety, phobic anxiety, and their physical equivalents. Importantly, the current conventional standard of therapy for hypo PT treatment did not restore well-being in these patients.

It is often desirable to extend the release time of an injected drug to increase its duration of action, or to reduce its toxic effects. Formulations that are readily soluble in the body are usually absorbed rapidly and provide a sudden burst of available drug as opposed to a more desirable and gradual release of the pharmacologically active product.

A variety of attempts have been made to provide controlled and extended-release pharmaceutical compounds, but previously disclosed techniques have not succeeded in overcoming all of the problems associated with the technology, such as achieving an optimal extended release time, maximizing stability and efficacy, reducing toxicity, maximizing reproducibility in preparation, and eliminating unwanted physical, biochemical, or toxicological effects introduced by undesirable matrix materials. Accordingly, there is a need for formulations that safely and efficaciously extend the half-life of existing pharmaceuticals and improve their therapeutic index from dose to dose and from patient to patient. This is of utmost importance in drugs that have a narrow therapeutic index to separate the dose for efficacy from that of toxicity. PTH much like insulin or thyroid hormone is recognized as such a drug where slightly excessive elevation in calcium beyond the physiological range has acute and chronic adverse consequences.

Mechanisms for providing extended release and an enhanced therapeutic index include sequestering molecules at the injection site or the use of prodrug derivative forms of the pharmaceutical, wherein the prodrug derivative is designed to delay onset of action and extend the half-life of the drug. The delayed onset of action is advantageous in that it allows systemic distribution of the prodrug prior to its activation. Accordingly, the administration of prodrugs can eliminate complications caused by peak activities upon administration and increase the therapeutic index of the parent drug.

Furthermore, receptor recognition and subsequent processing of peptide and protein agonists is the primary route of degradation of many peptide and protein-based drugs. Thus, binding of the peptide drug to its receptor will result in biological stimulation but will also initiate the subsequent deactivation of the peptide/protein induced pharmacology through the enzymatic degradation of the peptide or protein. Therefore, the use of prodrugs can also delay the time of action for administered drugs to allow an even distribution throughout the body prior to activation. The present disclosure provides compositions and methods to safely extend the biological action of PTH while minimizing excessive action shortly after administration.

### SUMMARY

In accordance with the present disclosure, PTH peptides can be modified to prevent their interaction with their corresponding receptor. More particularly, as disclosed herein PTH peptides can be reversibly modified by the linkage of a non-enzymatic self-cleaving dipeptide to the drug to form a complex that functions either as a depot composition, to localize the drug at the injection site for release in a controlled manner, and/or as a prodrug that is distributed throughout the body, but incapable of interacting with its receptor. Furthermore, such prodrug derivatives of PTH peptides can be further modified by the covalent linkage of a fatty-acyl or diacid group to the PTH peptide to enhance retention time and extend the retention of the prodrug and the duration of action of the underlying peptide upon cleavage of the prodrug moiety.

Advantageously, the PTH conjugates of the present disclosure safely extend the biological action of PTH while minimizing excessive action shortly after administration. Thus, the compositions disclosed herein provide a patient the ability to safely normalize serum calcium without the danger of excessive elevation of calcium that can have life-altering consequences. The technology increases the convenience in administration of the drug which should lead to increased compliance with therapy. As disclosed herein the PTH analogs of the present disclosure demonstrate extended duration of action allowing once-weekly administration to patients. However daily administration to patients is also envisioned as a method of allowing even more precise control of patient serum calcium levels.

The compositions of the present disclosure can be administered using standard routes such as subcutaneous administration. In one embodiment the composition is formulated for oral delivery by coformulation of the PTH conjugates in the present disclosure with an absorption enhancer, which can sufficiently augment the absorption of the PTH conjugate. Sodium N-[8-(2-hydroxybenzoyl)amino]caprylate (SNAC) is a delivery agent that has been reported to enhance the permeability of a diverse spectrum of molecules, including peptides, such as insulin, GLP-1, calcitonin and other macromolecules such as heparin. In accordance with one embodiment pharmaceutical compositions are provided for oral delivery wherein the composition comprises a PTH conjugate of the present disclosure and SNAC, optionally wherein the pharmaceutical composition is formulated as a tablet.

In accordance with one embodiment an acylated, conjugated derivative of parathyroid hormone is provided wherein the derivative has an improved therapeutic index and an in vivo extended time of action when administered to a warm-blooded mammal including, for example, Homo sapiens. In some embodiments, the present invention provides an acylated 31 amino acid PTH peptide further modified by covalent linkage of a self-cleaving dipeptide via an amide bond. In some embodiments, the present invention provides an acylated 32 amino acid PTH peptide further modified by covalent linkage of a self-cleaving dipeptide via an amid bond. In some embodiments, the present invention provides an acylated 33 amino acid PTH peptide further modified by covalent linkage of a self-cleaving dipeptide via an amid bond. In some embodiments, the present invention provides an acylated 34 amino acid PTH peptide further modified by covalent linkage of a self-cleaving dipeptide via an amid bond. In some embodiments, the present invention provides an acylated 38 amino acid PTH peptide further modified by covalent linkage of a self-cleaving dipeptide via an amid bond. More particularly, in one embodiment the modified parathyroid hormone (PTH) is a 33, 34 or 35 amino acid peptide of SEQ ID NO: 2, 3 or 30, respectively, wherein the PTH peptide is further modified by covalent linkage of a self-cleaving dipeptide via an amide bond, optionally at the N-terminal alpha amine of the PTH peptide.

In one embodiment the PTH peptide comprises an amino acid sequence selected from the group consisting of:

(SEQ ID NO: 133)
SVSEIQLMHX₁₀LGX₁₃HLX₁₆SX₁₈ERVEWLRX₂₆X₂₇LQDX₃₁H-Z,;
(SEQ ID NO: 134)
SVSEIQLMHX₁₀LX₁₂KHLX₁₆X₁₇X₁₈ERVEWLRKKLQDVH-Z;;
(SEQ ID NO: 135)
SVSEIQLMHX₁₀LGKHLX₁₆SX₁₈ERVEWLRKKLQDVH-Z
and
(SEQ ID NO: 7)
SVSEIQLMHNLGKHLNSMERVEWLRKKLQDVH-Z;

wherein Z is X₃₃, X₃₃F, X₃₃FX₃₅, X₃₃FVX₃₅ (SEQ ID NO: 136), X₃₃FVAX₃₅ (SEQ ID NO: 137), X₃₃FVALX₃₅ (SEQ ID NO: 138), X₃₃FVALGX₃₅ (SEQ ID NO: 139), X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally wherein Z is X₃₃, X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143);
X₁₀ and X₁₆ are independently Asp, Gln or Asn;
X₁₂ is Gly or Aib;
X₁₇ is amino isobutyric acid (Aib) or Ser;
X₁₈ is Met, Met(O), Leu, or Nleu;
X₁₃, X₂₆, and X₂₇ are independently selected from the group consisting of Arg, Glu, Asp and Lys;
X₃₁ is Gly or Val;
X₃₃ and X₃₅ each comprise an acylated amino acid comprising a C16-C30 fatty acid or C16-C30 diacid covalently linked to the side chain of the amino acid, optionally via a spacer, optionally wherein the acylated amino acid is selected from the group consisting of Lys, dLys, ornithine, Cys and homocysteine;
X₅₃ is Gln or Asn, optionally with the proviso that no more than one of X₁₂, X₁₆ and X₁₇ is Aib, and optionally wherein the C-terminal amino acid is modified to replace the carboxy terminus with an amide. In some embodiments the PTH peptide further comprises a self-cleaving dipeptide covalently bound to said PTH peptide via an amide bond, optionally wherein the self-cleaving dipeptide is covalently linked to the N-terminal alpha amine of the PTH peptide. In one embodiment the self-cleaving dipeptide comprises the structure A-B where
A is an amino acid, optionally an acylated amino acid comprising a C16-C30 fatty acid or C16-C30 diacid covalently linked to the side chain of the amino acid, optionally via a spacer; and
B is an N-alkylated amino acid. In certain embodiments the PTH peptide comprises an epsilon-acylated-Lys, epsilon-acylated-dLys, ornithine, epsilon-acylated ornithine, cysteine, S-acylated cysteine, homocysteine or S-acylated homocysteine, optionally wherein the acylated amino acid is the C-terminal amino acid of the PTH peptide.

In some embodiments the PTH peptide further comprises a non-natural amino acid. In some embodiments the PTH peptide comprises 1, 2, or 3 amino acid substitutions. In some embodiments the amino acid substitutions are conservative substitutions. In some embodiments the substitutions are made with non-conservative amino acids. In some embodiments the PTH peptides of the present invention include one or more non-natural amino acids. Non-limiting examples of non-natural amino acids for use in a PTH peptide of the present invention include phenylalanine derivatives containing benzophenone, ketone, iodide, or azide substituents; O-propargyltyrosine; α-aminocaprylic acid, O-methyl tyrosine, O-nitrobenzyl cysteine; 3-(naphthalene-2-ylamino)-2-amino-propanoic acid; para-substituted phenylalanine derivatives such as p-aminophenylalanine and p-methoxyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronophenylalanine; o-nitrobenzyltyrosine; amide and carbamate substituted lysines such as 2-amino-6-((R)-tetrahydrofuran-2-carboxamido)hexanoic acid, N-ε-D-prolyl-L-lysine, and N-ε-cyclopentyloxycarbonyl-L-lysine; N-ε-Acryloyl-L-lysine; N-ε-[(1-(6-nitrobenzo[d][1,3]dioxol-5-yl)ethoxy)carbonyl]-L-lysine; azido alanine; 2-(4′-pentenyl)alanine; alaninal; and N-ε-(1-methylcyclopro-2-enecarboxamido)lysine.

In one embodiment a PTH conjugate is provided comprising any of the PTH peptides disclosed herein and a self-cleaving dipeptide covalently bound to said PTH peptide via an amide bond, optionally to the N-terminal alpha amine of the PTH peptide. In one embodiment the conjugate comprises a PTH peptide having an amino acid sequence selected from the group consisting of

(SEQ ID NO: 4)
SVSEIQLMHNLX₁₂X₁₃HLX₁₆X₁₇MERVEWLRX₂₆X₂₇LQDX₃₁H-Z,
(SEQ ID NO: 5)
SVSEIQLMHNLGX₁₃HLNSMERVEWLRX₂₆X₂₇LQDX₃₁H-Z,
(SEQ ID NO: 6)
SVSEIQLMHNLX₁₂KHLX₅₆X₁₇MERVEWLRKKLQDVH-Z;
(SEQ ID NO: 135)
SVSEIQLMHX₁₀LGKHLX₁₆SX₁₈ERVEWLRKKLQDVH-Z;
and
(SEQ ID NO: 7)
SVSEIQLMHNLGKHLNSMERVEWLRKKLQDVH-Z

wherein
Z is X₃₃F, X₅₃FX₃₅, X₃₃FX₃₅, or X₃₃, optionally Z is X₅₃FX₃₅ or X₃₃;
X₁₀ and X₁₆ are independently Asp, Gln or Asn;
X₁₂ is amino isobutyric acid (Aib) or Gly;
X₅₆ is amino isobutyric acid (Aib) or Asn;
X₁₇ is amino isobutyric acid (Aib) or Ser;
X₁₈ is Met, Met(O), or Nleu;
X₁₃, X₂₆, and X₂₇ are independently selected from the group consisting of Arg, Glu, Asp and Lys, optionally wherein X₁₃, X₂₆, and X₂₇ are independently selected from Glu and Lys;
X₃₁ is Gly or Val;
X₃₃ and X₃₅ each comprise an acylated amino acid;
X₅₃ is Gln, Asp, Glu, or Asn, optionally wherein X₅₃ is Asn,

optionally with the proviso that only one of X₁₂, X₁₆ and X₁₇, is Aib; wherein
the self-cleaving dipeptide of the conjugate comprises the general structure A-B—;

wherein

A is an acylated amino acid; and

B is an N-alkylated amino acid;

wherein said acylated amino acid of each of X₃₃, X₃₅ and A is an amino acid comprising a C16-C30 fatty acid or C16-C30 diacid covalently linked to its amino acid side chain, optionally via a spacer, and wherein the self-cleaving dipeptide is linked to said PTH peptide through formation of an amide bond between B and a primary amine of the PTH peptide, optionally wherein the primary amine is located on a side chain of a lysine substitution present at position 13, 16, 19, 22, or 26, or the N-terminal alpha amine of the PTH peptide. In a further embodiment amino acid “A” of the self-cleaving dipeptide is a lysine residue acylated with a C16-C30 fatty acid or C16-C30 diacid. In one embodiment A and B are selected to provide a chemical cleavage half-life (t½) of A-B from said PTH peptide of at least about 24 hours to about 240 hours, about 48 hours to about 168 hours, about 48 to about 120 hours, or about 70 to about 120 hours, about 80 to about 120 hours, about 90 to about 120 hours, or about 100 to about 120 hours in standard PBS solution under physiological conditions. In one embodiment the C-terminal amino acid of any of the PTH conjugates disclosed herein can be modified to replace the native carboxyl group with an amide.

In accordance with one embodiment a conjugate derivative of PTH is provided wherein a self-cleaving dipeptide is covalently bound to the N-terminal alpha amine of said PTH peptide via an amide bond, further wherein the PTH peptide comprises the sequence of SVSEIQLMHNLGKHLNSMERVEWLRKKLQDVHX₃₃-Z (SEQ ID NO: 2), SVSEIQLMHNLGKHLNSMERVEWLRKKLQDVHFNX₃₅ SEQ ID NO: 15) or SVSEIQLMHNLGKHLNSMERVEWLRKKLQDVHX₃₃ SEQ ID NO: 2), wherein
X₃₃ and X₃₅ are each independently an amino acid wherein the side chain of the amino acid is acylated with a C16-C20 fatty acid or C16-C20 diacid, optionally wherein X₃₃ and X₃₅ are independently selected from C16-C20 acylated lysine, C16-C20 acylated ornithine, C16-C20 acylated cysteine and C16-C20 acylated homocysteine, optionally wherein X₃₃ and X₃₅ are both a C16-C20 acylated Lys; Z is selected from the group consisting of F-R, FV-R FVA-R, FVAL-R (SEQ ID NO: 147), FVALG-R (SEQ ID NO: 148), and FVALGA-R (SEQ ID NO: 149), wherein R is COOH or CONH₂, and said self-cleaving dipeptide is a dipeptide of the structure:

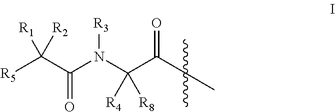

wherein
R₁, is a side chain selected from the group consisting of C₁-C₁₈ alkyl, (C₁-C₄ alkyl)OH, (C₁-C₄ alkyl)SH, (C₁-C₄ alkyl)COOH, and (C₁-C₄ alkyl)NH₂, optionally wherein a C16-C20 fatty acid or a C16-C20 diacid is covalently linked to said side chain;
R₂, R₄ and R₈ are independently H, or C₁-C₄ alkyl;
R₃ is C₁-C₄ alkyl, or R₄ and R₃ together with the atoms to which they are attached form a 5- or 6-member heterocyclic ring, including e.g., a pyrrolidine ring; and
R₅ is NH₂, with the proviso that when R₄ and R₃ together with the atoms to which they are attached form a 5- or 6-member heterocyclic ring, including e.g., a pyrrolidine ring, R₂ is not H. In one embodiment the acylated amino acid of A, X₃₃ and X₃₅ is independently selected from an amino acid having the general structure of

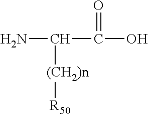

wherein n is an integer selected from the range of 1-4 and R₅₀ is selected from the group consisting of NH—CO(CH₂)₁₄₋₂₀COOH, NH-[spacer]-CO(CH₂)₁₄₋₂₀COOH, S(CH₂)₁₄₋₂₀COOH, S-[spacer]-CO(CH₂)₁₄₋₂₀COOH, N═N═N-[spacer]-CO(CH₂)₁₄₋₂₀COO, HC [symbol] C-[spacer]-CO(CH₂)₁₄₋₂₀COO, and CHO-[spacer]-CO(CH₂)₁₄₋₂₀COO. In one embodiment the acylated amino acid of A, X₃₃ and X₃₅ is independently selected from lysine, d-lysine, ornithine, cysteine, homocysteine, azidoalanine, 2-(4′-pentenyl)alanine, or alaninal wherein the side chain of said acylated amino acid is covalently linked to a C16-C22 fatty acid or C16-C22 diacid optionally through a spacer comprising an amino acid or dipeptide. In one embodiment the spacer comprises a gamma glutamic acid. In one embodiment the optional spacer comprises two gamma glutamic acids, optionally wherein the two gamma glutamic acids are joined to one another via an intervening functionalized PEG polymer, [COCH₂(OCH₂CH₂)ₖHN]q, wherein k and q are each integers independently selected from 1, 2, 3, 4, 5, 6, 7 or 8. In one embodiment the spacer is -{gamma glutamic acid-[COCH₂(OCH₂CH₂)₂NH—COCH₂(OCH₂CH₂)₂]NH-gamma glutamic acid}-. In a further embodiment the self-cleaving dipeptide has the structure of formula I wherein R₁, is (C₁-C₄ alkyl)NH—CO(CH₂)₁₄₋₂₀COOH or (C₁-C₄ alkyl)NH-[spacer]-CO(CH₂)₁₄₋₂₀COOH; R₂ and R₈ are each H; R₄ is H, or CH₃; R₃ is CH₃ and R₅ is NH₂, optionally wherein the first amino acid of the self-cleaving dipeptide is an amino acid in the D-stereochemical configuration and the spacer is selected from the group consisting of a gamma glutamic acid, a gamma glutamic acid-gamma glutamic acid dipeptide, and a gamma glutamic acid-[COCH₂(OCH₂CH₂)ₖHN]q-gamma glutamic acid, wherein k is an integer selected from the range of 1-8 or 2-4 and q is 1, 2, 4 or 8. In one embodiment k is 2 or 4 and q is 1 or 2.

In accordance with one embodiment a pharmaceutical composition is provided comprising any of the novel PTH conjugates disclosed herein, preferably at a purity level of at least 90%, 91%, 92%, 93%, 94%, 95%, 96%, 97%, 98% or 99%, and a pharmaceutically acceptable diluent, carrier or excipient. Such compositions may contain a PTH conjugate as disclosed herein at a concentration of at least 0.1-10 mg/ml, or higher. In one embodiment the pharmaceutical compositions comprise aqueous solutions that are sterilized and optionally stored within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

In accordance with one embodiment an improved method of treating hypoparathyroidism in patients in need thereof is provided. The method comprises the steps of administering a PTH conjugate of the present disclosure in an amount therapeutically effective for the control of hypoparathyroidism. In one embodiment PTH peptide is acylated with a fatty acid or diacid group of sufficient size to bind serum albumin with high affinity, and further wherein the PTH peptide is linked to a self-cleaving dipeptide wherein an amino acid of the dipeptide is optionally acylated with a fatty acid or diacid group of sufficient size to bind serum albumin with high affinity.

In accordance with one embodiment an improved method of treating osteoporosis or osteopenia in patients in need thereof is provided. The method comprises the steps of administering a PTH conjugate of the present disclosure in an amount therapeutically effective for the control of serum calcium levels. In one embodiment PTH peptide is acylated with a fatty acid or diacid group of sufficient size to bind serum albumin with high affinity, and further modified by linkage to a self-cleaving dipeptide wherein an amino acid of the dipeptide is optionally acylated with a fatty-acyl group of sufficient size to bind serum albumin with high affinity.

### BRIEF DESCRIPTION OF THE DRAWINGS

FIG. 1 is a graph showing the effect of Natpara® (SEQ ID NO: 1) on calcium urinary excretion rates in humans. Plasma PTH levels and calcium urinary excretion rates over time are shown after subcutaneous administration of 100 ug dose of Natpara®. Although Natpara® is effective in reversing calcium loss, the compound has an insufficient duration of action resulting in adequate control of serum calcium levels being limited to only ⅓ of the day with excessive highs and lows during the remaining time.

FIGS. 2A-2B demonstrates the use of a dipeptide to form a prodrug of PTH. FIG. 2A is a schematic drawing of the reaction that cleaves an amide linked dipeptide from a PTH conjugate to form a biologically activate PTH peptide and a diketopiperazine (DKP). FIG. 2B demonstrates chemical cleavage is a linear, zero-order reaction that is concentration-independent and requires no additional components (enzymes, or catalysts). The speed of the reaction at a specific pH and temperature is a function of the specific dipeptide and can be adjusted from less than 30 minutes to more than 500 hours.

FIGS. 3A & 3B show the ability of PTH analogs to stimulate the PTH receptor in PTH Receptor-1 stably transfected cells with a luciferase reporter of cAMP production. FIG. 3A provides data for SEQ ID Nos: 9, 10, 11 and 12 while FIG. 3B presents data for SEQ ID Nos: 9, 12, 13 and 14. The amino acid sequences of the PTH analogs are provided below. The data demonstrates that PTH analogs wherein a 35 amino acid PTH peptide is covalently modified by addition of an acylated lysine (having a C18 fatty diacid chain linked to its side chain) at position 35 retains high potency at the PTH receptor (SEQ ID NO: 12) whereas attachment of an acylated dipeptide (SEQ ID Nos: 10, 11, and 14) to PTH (1-34) decreases the potency, and the combination in attachment of a acylated dipeptide at the N-terminus and acylation at the carboxy terminal amino acid is also of low potency (SEQ ID NO: 13).

SVSEIQLMHNLGKHLNSMERVEWLRKKLQDVHNF (SEQ ID NO: 9);
(dK)K(γE-COC₁₆H₃₂CO₂H)GSVSEIQLMHNLGKHLNSMERVEWLRK
KLQDVHNF (SEQ ID NO: 10);
(dK)(γE-COC₁₆H₃₂CO₂H)(N-Me)GSVSEIQLMHNLGKHLNSMERV
EWLRKKLQDVHNF(SEQ ID NO: 11) ;
SVSEIQLMHNLGKHLNSMERVEWLRKKLQDVHNFK
(γE-COC₁₆H₃₂CO₂H)-NH2 (SEQ ID NO: 12);
(dK)(γE-COC₁₆H₃₂CO₂H)(N-Me)GSVSEIQLMHNLGKHLNSMERVE
WLRKKLQDVHNFK(γE-COC₁₆H₃₂CO₂H)-NH2 (SEQ ID NO: 13).
(dK)(γE-COC₁₆H₃₂CO₂H)(N-Me)GSVSEIQLMHNLGKHLNSMERVE
WLRKKLQDVHQF-OH (SEQ ID NO: 14).

FIG. 4 show the ability of 33 amino acid PTH analogs (comprising the PTH sequence of SEQ ID NO: 2) to stimulate the PTH receptor in PTH Receptor-1 stably transfected cells with a luciferase reporter of cAMP production. SEQ ID NO: 18 is a PTH analog comprising an alanine substitution at position 8 that decreases PTH activity at the PTH receptor. The data demonstrates that, similar to the 34 amino acid PTH analogs, addition of a fatty acid chain to the C-terminus of a 33 amino acid PTH analog retains the high potency of the parent compound (SEQ ID NO: 16) but the combination of a dipeptide with fatty-acylation at the carboxy terminus decreases the potency (SEQ ID NO: 17):

PTH (1-34; SEQ ID NO: 9)
PTH(1-33), K33(γE-2xOEG-γE-diacidC18) (SEQ ID NO: 16)
PTH(1-33), K33(γE-2xOEG-yγE-diacidC18) with N-terminal dipeptide
extension of dKN(Me)G (SEQ ID NO: 17)
PTH(1-33), A8, K33(γE-2xOEG-γE-diacidC18) (SEQ ID NO: 18)

FIGS. 5A-5C are graphs demonstrating the in vivo efficacy in mice for PTH analogs comprising an acylated amino acid at the C-terminus of the PTH peptide to increase serum calcium levels (FIG. 5A) and reduce serum phosphate (FIG. 5B) in a dose-responsive manner over an extended time period of 72 hours. SVSEIQLMHNLGKHLNSMERVEWLRKKLQDVHK(γE-COC₁₆H₃₂CO₂H)F—OH (SEQ ID NO: 102). FIG. 5C is a graph demonstrating the in vivo efficacy in mice of additional acylated PTH analogs (PTH(1-34), K33(γE-diacidC18) (SEQ ID NO: 102); PTH(1-33), K33(γE-diacidC18) (SEQ ID NO: 74); and PTH(1-33), K33(γE-(miniPEG)₂-γE-diacidC18) (SEQ ID NO: 77) to increase serum calcium levels.

FIGS. 6A and 6B represents results from pharmacokinetic (PK) studies to determine appearance and disappearance times in mice using PTH-analogs designed for sustained duration, but incapable of being converted to active drug. Each compound was administered subcutaneously at a dose of 100 nmol/kg SC in mice. FIG. 6A shows the data for (dK)GSVSEIQLMHNLGKHLNSMERVEWLRKKLQDVHK (γE-(miniPEG)₂-γE-COC₁₆H₃₂CO₂H)—OH (SEQ ID NO: 78); (dK)GSVSEIQLMHNLGKHLNSMERVEWLRKKLQDVHK (γE-(miniPEG)₂-γE-COC₁₈H₃₂CO₂H)—OH (SEQ ID NO: 84); and (dK)(γE-(miniPEG)₂-γE-COC₁₆H₃₂CO₂H)GSVSEIQLMHNLGKHLNSMERVEWLRKKLQDVHK (γE-(miniPEG)₂-γE-COC₁₆H₃₂CO₂H)—OH (SEQ ID NO: 79). FIG. 6B shows the same results as in FIG. 6A for SEQ ID Nos: 78, 84 and 79 but expressed in logarithmic form. FIGS. 6A and 6B demonstrate the importance of fatty acyl length and the difference between acylated and diacylated PTH analogs of the present invention.

FIGS. 7A and 7B represent results from pharmacokinetic (PK) studies to determine retention time in monkeys administered a single subcutaneous dose of a prodrug PTH-analog at 25 nmol/kg wherein the PTH peptide has been modified with an alanine substitution at position 8 to create a low potency PTH analog. FIG. 7A is a graph demonstrating the detected levels of the prodrug PTH analog SEQ ID NO: 94: PTH(1-33), A8, K33(γE-2×OEG-γE-diacidC18) dK₋₁, N(Me)G₀; and its activated form SEQ ID NO: 93: PTH(1-33), A8, K33(γE-2×OEG-γE-diacidC18) overtime after administration of a single dose of the prodrug (SEQ ID NO: 94). FIG. 7B is a graph demonstrating the detected levels of the prodrug PTH analog SEQ ID NO: 95: PTH(1-33), A8, K33(γE-2×OEG-γE-diacidC18) dK⁻¹(γE-2×OEG-γE-diacidC18), N(Me)G⁰ and its activated form: SEQ ID NO: 93: PTH(1-33), A8, K33(γE-2×OEG-γE-diacidC18) over time after administration of a single dose of the prodrug (SEQ ID NO: 95). The data demonstrate accumulation of the active form over time, corresponding with a decrease of the prodrug form, resulting in a relatively consistent amount of active form over an extended time-period. The doubly fatty acylated prodrug (SEQ ID: 95) achieves a higher concentration that is sustained for a longer period than the singly fatty acylated prodrug (SEQ ID: 94).

FIG. 7C is the same drug concentration results presented in FIG. 7A presented in a logritmetic scale.

FIG. 7D is the same drug concentration results presented in FIG. 7B presented in a logritmetic scale

FIGS. 8A-8C show the functional response of cyclic AMP production in CHO-K1 PTHR1 cells used in the cAMPHunter Teriparitide Bioassay, described in Example 11, in response to treatment, in FIG. 8A with PTH(1-34), a PTH agonist of the present invention SEQ ID NO: 77; in FIG. 8B with a non-cleavable prodrug SEQ ID NO: 79; in FIG. 8C with PTH(1-34), and a cleavable prodrug SEQ ID NO: 87.

FIGS. 9A-9F show LCMS results of a PTH prodrug of the present invention, SEQ ID NO: 87, to its active drug form, SEQ ID NO: 77, in PBS buffer over the course of 8 days. The 7.3-minute peak shows prodrug and in FIG. 9A, at 0 days, that is the only peak. In FIG. 9B, at one day, there is the beginning of an active drug peak at 6.5 minutes. In FIGS. 9C and 9D, 2 days and 3 days respectively, there is still less active drug than prodrug. In FIGS. 9E and 9F, at 5 and 8 days respectively, there is more active drug than prodrug.

FIG. 10 is a graph of the LCMS assay data from Example 11 fitted to zero-order reaction kinetics. The LCMS assay shows the conversion of prodrug SEQ ID NO: 87 to active drug SEQ ID NO: 77 over 192 hours and shows a half-life of 112 hours with a high linear correlation (R>0.99).

FIG. 11 shows the functional response of cyclic AMP production in CHO-K1 PTHR1 cells from the cAMPHunter Teriparitide Bioassay following varying incubation lengths, from 0 to 8 days of treatment as assessed in FIG. 9A-9F, with peptides of the present invention SEQ ID NO: 87, SEQ ID NO: 77 or with PTH(1-34).

FIG. 12 shows the functional response of cyclic AMP production in CHO-K1 PTHR1 cells from the cAMPHunter Teriparitide Bioassay following treatment with peptides SEQ ID NO: 77, SEQ ID NO: 109, and SEQ ID NO: 110.

FIGS. 13A and 13B shows the functional response of cyclic AMP production in CHO-K1 PTHR1 cells from the cAMPHunter Teriparitide Bioassay following treatment with peptides, in FIG. 13A, SEQ ID NO: 77, SEQ ID NO: 118, and SEQ ID NO: 120; and in FIG. 13B, SEQ ID NO: 77, SEQ ID NO: 122, and SEQ ID NO: 124.

FIGS. 14A and 14B are graphs of the absolute serum calcium levels (FIG. 14A) and relative changes in calcium levels (FIG. 14B) in mice following treatment with either vehicle, 20 or 40 nmol/kg of PTH analog SEQ ID NO: 77 over a time period of 48 hours.

FIG. 15 shows the serum concentration in a rat over a one week period after administration of prodrug SEQ ID NO: 87 and active drug SEQ ID NO: 77. A single dose by subcutaneous injection was given, either of prodrug or of active drug, and serum concentration measurements were taken over the course of a week. The amount of active drug derived from prodrug was also measured (black dashed line, square symbol).

FIGS. 16A and 16B shows the absolute serum calcium concentration and its change in a rat over the period of a week after subcutaneous dosing of prodrug SEQ ID NO: 87 at 30 or 60 nmol/kg.

FIGS. 17A and 17B shows the absolute serum phosphate concentration and its change in a rat over the period of a week after subcutaneous dosing of prodrug SEQ ID NO: 87 at 30 or 60 nmol/kg.

FIGS. 18A and 18B are graphs of the change in serum calcium levels (FIG. 18A) and serum phosphate levels (FIG. 18B) in Sprague-Dawley rats following daily subcutaneous injections with either vehicle, 20 nmol/kg active drug SEQ ID NO: 77, 20 nmol/kg or 40 nmol/kg prodrug SEQ ID NO: 87.

FIGS. 19A and 19B are graphs of serum concentration of prodrug SEQ ID NO: 87 (FIG. 19A) and active drug SEQ ID NO: 77 (FIG. 19B) in rats following seven daily subcutaneous injections beginning on day 0 with measurements at different time points over 144 hours. There is a 4-fold difference in total concentration of prodrug to drug.

FIGS. 20A and 20B are graphs showing the calcium levels in rodents treated with repeated 28 daily doses of vehicle starting on day 0, 4, 8, and 12 nmol/kg prodrug SEQ ID NO: 87 over 28 days (FIG. 20A) and the washout calcium levels following last dose on day 28 (FIG. 20B).

FIG. 21 is a pharmacokinetic analysis by LCMS of the conversion of prodrug to drug from varying starting doses (4, 8, or 12 nmol/kg) of prodrug SEQ ID NO: 87 in rats with repeated dosing over 28 days as detailed in Example 5. The solid lines show the plasma concentration in nM of prodrug while the corresponding dashed line is the plasma concentration of active drug SEQ ID NO: 77.

FIG. 22 is a pharmacokinetic analysis by LCMS of a smaller time portion of FIG. 21, and it shows the plasma concentration levels of active drug (solid bars) and prodrug (white dotted bars) following the stop of repeat dosing from Example 15 and the change in plasma concentrations in the hours (2, 7, and 24) following last dose.

FIG. 23 is a graph of the calcium plasma concentration in disease model rats with a control rat surgically manipulated (sham), and others with a surgical parathyroidectomy where they are then treated with either vehicle or 10, 25, or 40 nmol/kg prodrug SEQ ID NO: 87. Doses were given prior to each time measurement taken at time 0, 24, 48, 72 hours, which was the last dose, and an additional measurement taken at 144 hours, 72 hours following the last dose.

FIG. 24 is a graph showing the serum calcium levels in disease model rats with a sham control as described in Example 5. Rats were dosed with vehicle or varying levels of compound, 10, 25, and 40 nmol/kg prodrug SEQ ID NO: 87 dosed daily over 10 days and determination over 25 days.

FIG. 25 is a graph showing the serum phosphate levels in disease model rats with a sham control as described in Example 5. Rats were dosed with vehicle or varying levels of compound, 10, 25, and 40 nmol/kg prodrug SEQ ID NO: 87 dosed daily over 10 days and determination over 25 days.

FIGS. 26A and 26B are graphs of serum concentration of prodrug SEQ ID NO: 87 (FIG. 26A) and active drug SEQ ID NO: 77 (FIG. 26B) in monkeys following subcutaneous dosing with varying concentrations of prodrug SEQ ID NO: 87 (2.5, 3.75, and 5.0 nmol/kg). There is an approximate 4-fold difference of prodrug to drug.

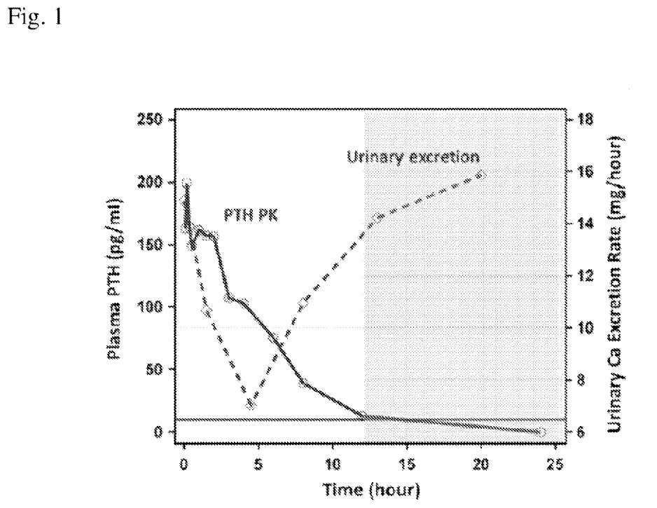

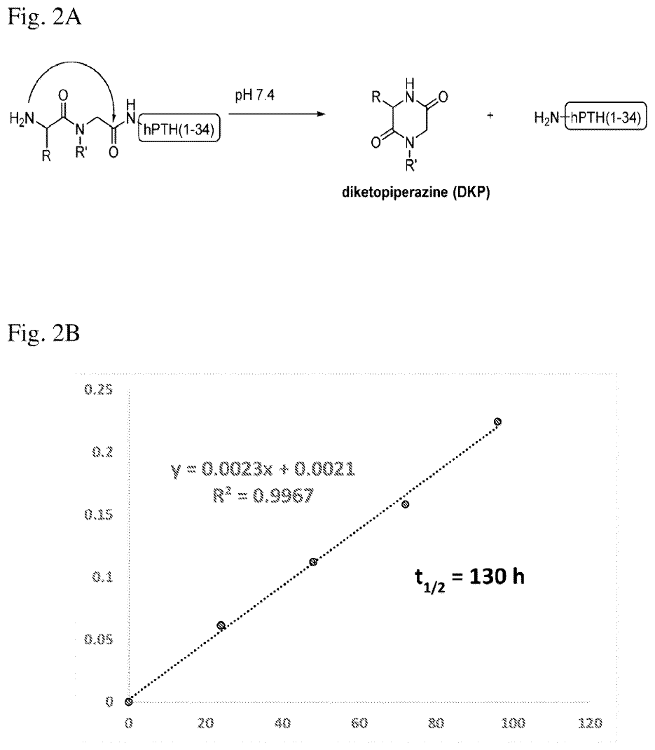

### DETAILED DESCRIPTION

### Definitions

In describing and claiming the invention, the following terminology will be used in accordance with the definitions set forth below.

As used herein the term “PTH peptide” as used herein includes any peptide comprising, either the amino acid sequence of SEQ ID NO: 7, or any analog of the amino acid sequence of SEQ ID NO: 7, including amino acid substitutions, additions, deletions or post translational modifications (e.g., methylation, acylation, alkylation, PEGylation, and the like) of the peptide, wherein the analog stimulates increased calcium in the blood upon administration to a patient.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein the terms “native” or “natural” define a condition found in nature. A “native amino acid” is an amino acid present in nature that was produced by natural means.

As used herein the term “amino acid” encompasses any molecule containing both amino and carboxyl functional groups, wherein the amino and carboxylate groups are attached to the same carbon (the alpha carbon). The alpha carbon optionally may have one or two further organic substituents. An amino acid can be designated by its three-letter code, one letter code, or in some cases by the name of its side chain. For example, a non-canonical amino acid comprising a cyclohexane group attached to the alpha carbon is termed “cyclohexane” or “cyclohexyl.” For the purposes of the present disclosure designation of an amino acid without specifying its stereochemistry is intended to encompass either the L or D form of the amino acid, or a racemic mixture. However, in the instance where an amino acid is designated by its three-letter code (e.g., Lys) or single letter (e.g., K), such a designation is intended to specify the native L form of the amino acid, whereas the D form will be specified by inclusion of a lower-case d before the three-letter code or single code (i.e., dLys or dK). As used herein the designation of a specific amino acid is intended to encompass the native amino acid as well as any isotopically enriched derivative thereof that differs in molecular weight from the native amino acid but has equivalent physical and biological properties as the native amino acid.

As used herein the term “hydroxyl acid” refers to amino acids that have been modified to replace the alpha carbon amino group with a hydroxyl group.

As used herein the term “non-coded (non-canonical) amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

A “dipeptide” is the result of the linkage of an alpha amino acid or an alpha hydroxyl acid to another amino acid, through a peptide bond.

As used herein the term “chemical cleavage” absent any further designation encompasses a non-enzymatic reaction that results in the breakage of a covalent chemical bond.

A “bioactive peptide” refers to peptides which can exert a biological effect in vitro and/or in vivo. As used herein a general reference to a peptide is intended to encompass peptides that have modified amino and carboxy termini. For example, an amino acid sequence designating the standard amino acids is intended to encompass standard amino acids at the N- and C-terminus as well as a corresponding hydroxyl acid at the N-terminus and/or a corresponding C-terminal amino acid modified to comprise an amide group in place of the terminal carboxylic acid.

As used herein an “acylated” amino acid is an amino acid comprising an acyl group which is non-native to a naturally occurring amino acid, regardless of the means by which it is produced. Exemplary methods of producing acylated amino acids and acylated peptides are known in the art and include acylating an amino acid before inclusion in the peptide or peptide synthesis followed by chemical acylation of the peptide. In some embodiments, the acyl group causes the peptide to have one or more of (i) a prolonged half-life in circulation, (ii) a delayed onset of action, (iii) an extended duration of action, (iv) an improved resistance to proteases, such as DPP-IV, and (v) altered potency at a receptor for PTH.

As used herein, an “alkylated” amino acid is an amino acid comprising an alkyl group which is non-native to a naturally occurring amino acid, regardless of the means by which it is produced. Exemplary methods of producing alkylated amino acids and alkylated peptides are known in the art and including alkylating an amino acid before inclusion in the peptide or peptide synthesis followed by chemical alkylation of the peptide.

As used herein, the term “prodrug” is defined as any compound that undergoes chemical modification before exhibiting its pharmacological effects.

As used herein a “receptor” is a molecule that recognizes and binds with specific molecules in a high affinity interaction, producing some biological effect (either directly or indirectly) in a cell, or on the cells and/or tissues of the host organism. A “cellular receptor” is a molecule on or within a cell that recognizes and binds with specific molecules, producing some effect (either directly or indirectly) in the cell.

The term “identity” as used herein relates to the similarity between two or more sequences. Identity is measured by dividing the number of identical amino acid residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “PTH peptide” is directed to those peptides which have biological activity (as agonists or antagonists) at a receptor for native PTH and comprise an amino acid sequence that shares at least 70% sequence identity (e.g., 70%, 75%, 80%, 85%, 90%, 95%) with the peptide sequence of (SEQ ID NO: 7) in the aligned portion.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this disclosure the phrase “standard PBS” refers to a solution having have a final concentration of 137 mM NaCl, 10 mM Phosphate, 2.7 mM KCl, and a pH of 7.2-7.4.

As used herein the term “pharmaceutically acceptable salt” refers to salts of compounds that retain the biological activity of the parent compound, and which are not biologically or otherwise undesirable. Many of the compounds disclosed herein can form acid and/or base salts by virtue of the presence of amino and/or carboxyl groups or groups similar thereto.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a drug refers to a nontoxic amount but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as subcutaneous, intramuscular, intraspinal, or intravenous.

As used herein an amino acid “substitution” refers to the replacement of one amino acid residue by a different amino acid residue.

As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine (hCys)
V. Large, aromatic residues:
   Phe, Tyr, Trp, acetyl phenylalanine, napthylalanine (Nal)

As used herein the general term “polyethylene glycol chain” or “PEG chain”, refers to mixtures of condensation polymers of ethylene oxide and water, in a branched or straight chain, represented by the general formula H(OCH₂CH₂)ₖOH, wherein k is at least 2. As used herein the term “miniPEG” or “OEG” defines a functionalized polyethylene compound comprising the structure:

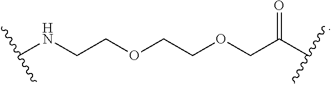

As used herein the term “pegylated” and like terms refers to a compound that has been modified from its native state by linking a polyethylene glycol chain to the compound. A “pegylated polypeptide” is a polypeptide that has a PEG chain covalently bound to the polypeptide.

As used herein a “linker” or “spacer” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties, and enzyme-cleavable groups.

As used herein a “dimer” is a complex comprising two subunits covalently bound to one another via a linker. The term dimer, when used absent any qualifying language, encompasses both homodimers and heterodimers. A homodimer comprises two identical subunits, whereas a heterodimer comprises two subunits that differ, although the two subunits are substantially similar with one another.

The term “C₁-Cₙ alkyl” wherein n can be from 1 through 6, as used herein, represents a branched or linear alkyl group having from one to the specified number of carbon atoms. Typical C₁-C₆ alkyl groups include, but are not limited to, methyl, ethyl, n-propyl, iso-propyl, butyl, iso-butyl, sec-butyl, tert-butyl, pentyl, hexyl and the like.

As used herein the term C16-C20 fatty acid designates the structure: —CO(CH₂)₁₅₋₁₈CH₃ and the term C16-C20 diacid designates the structure: —CO(CH₂)₁₄₁₈COOH, wherein the prefix “C16-C20” designates the variable total number of carbons in the compounds encompassed by the designation. For example, a C18 diacid represents the structure: —CO(CH₂)₁₆COOH. As used herein a generic reference to an acylated amino acid encompasses both an amino acid having its side chain acylated with a fatty acid and an amino acid having its side chain acylated with a diacid.

Physiological conditions as disclosed herein are intended to include a temperature of about 35 to 40° C. and a pH of about 7.0 to about 7.4, and more typically include a pH of 7.2 to 7.4 and a temperature of 36 to 38° C. Since physiological pH and temperature are tightly regulated in humans within a highly defined range, the speed of conversion from dipeptide/drug complex (prodrug) to drug will exhibit high intra and interpatient reproducibility.

As used herein the term “patient” without further designation is intended to encompass any warm-blooded vertebrate domesticated animal (including for example, but not limited to livestock, horses, cats, dogs and other pets) and humans and includes individuals not under the direct care of a physician.

### Abbreviations

Lower case k=d-isomer of lysine
γE=l-isomer of gamma, glutamic acid

(miniPEG)₂ = COCH₂OCH₂CH₂OCH₂CH₂NH

COC₁₆H₃₂CO₂H=(C18 diacid)
(N-Me)G=sarcosine

SVSEIQLMHNLGKHLNSMERVEWLRKKLQDVH (SEQ ID NO: 7) = PTH

Upper case K=l-isomer of lysine

### EMBODIMENTS

In accordance with one embodiment, improved analogs of parathyroid hormone (PTH) and a method of treating hypothyroidism and osteoporosis are provided. More particularly, the analogs of PTH disclosed herein have an improved therapeutic index and extended time of action relative to native PTH and known active fragments thereof.

In accordance with one embodiment a conjugated derivative of parathyroid hormone and conjugated derivatives of PTH-related peptide (PTHrp), are provided wherein the derivative has an in vivo extended time of action and improved therapeutic index, relative to the unmodified parent peptide, when administered to a warm-blooded mammal, including Homo sapiens. In one embodiment the conjugated derivatives comprise a self-cleaving dipeptide, optionally acylated, linked to the N-terminal alpha amine and acylation at the C-terminal amino acid. More particularly, in one embodiment the modified parathyroid hormone (PTH) is a 33, 34 or 35 amino acid peptide comprising the sequence of SEQ ID NO: 2, 3 or 30, respectively, or comprising an amino acid sequence having at least 85%, 90%, 95% or 97% sequence identity to SEQ ID NO: 2, 3, 7 or 30 in the aligned portion of the compared sequences, that has a fatty acid or diacid group (including for example a C16-C20 fatty acid or C16-C20 diacid) covalently linked to the side chain of an amino acid selected from position 13, 16, 19, 22, 26 and 33 (relative to SEQ ID NO: 7) or at the C-terminal amino acid, and optionally is further modified by the covalent linkage of a self-cleaving dipeptide. The self-cleaving dipeptide can be linked via an amide bond to any primary amine of the PTH peptide including the N-terminal alpha amine or any primary amine bearing side chain of the peptide, including for example at an amino acid at a position selected from 13, 16, 19, 22, 26 and 33 (relative to SEQ ID NO: 7). The side chains of amino acids comprising the self-cleaving dipeptide can be optionally linked to a fatty acid or diacid group or other polymer to more effectively block activity of the PTH peptide until removal of the self-cleaving dipeptide through a non-enzymatic self-cleaving mechanism. In one embodiment the first amino acid of the dipeptide has its side chain acylated with a C16-C20 fatty acid or C16-C20 diacid.

In one embodiment the self-cleaving dipeptide is covalently linked via an amide bond to the amino terminus of the PTH peptide, optionally at the N-terminal alpha amine, and further comprises an acylated amino acid at one or more positions selected from the group consisting of position 13, 16, 19, 22, 26 and 33 (relative to SEQ ID NO: 7) or as the C-terminal amino acid. In one embodiment the modified PTH peptide of the disclosed PTH conjugates comprises a sequence selected from the group consisting of

(SEQ ID NO: 7)
SVSEIQLMHNLGKHLNSMERVEWLRKKLQDVH,
(SEQ ID NO: 31)
SVSEIQLMHNLGKHLNSMERVEWLRKKLQDVHN,
(SEQ ID NO: 32)
SVSEIQLMHNLGKHLNSMERVEWLRKKLQDVHNF,
(SEQ ID NO: 33)
SVSEIQLMHNLGKHLNSMERVEWLRKKLQDVHNFV,
(SEQ ID NO: 34)
SVSEIQLMHNLGKHLNSMERVEWLRKKLQDVHNFVA,
(SEQ ID NO: 35)
SVSEIQLMHNLGKHLNSMERVEWLRKKLQDVHNFVAL,
(SEQ ID NO: 36)
SVSEIQLMHNLGKHLNSMERVEWLRKKLQDVHNFVALG,
and
(SEQ ID NO: 37)
SVSEIQLMHNLGKHLNSMERVEWLRKKLQDVHNFVALGA,

wherein the PTH peptide is modified by the covalent linkage of a self-cleaving dipeptide, optionally at the N-terminal alpha amine of the PTH peptide and the linkage of an acyl group, of sufficient size (e.g. C14-C22 fatty acid or C14-C22 diacid) to bind serum albumin, to the side chain of an amino acid at position 13, 16, 19, 22, 26, 33 or at the C-terminal portion of the peptide of any one of SEQ ID Nos: 7, 31-37, or the corresponding position of any analogs thereof, optionally wherein the peptides of SEQ ID Nos: 7, 31-37 comprises a substitution of an acylated lysine residue at one or two of positions selected from 13, 16, 19, 22, 26, 33 and the C-terminal amino acid. In one embodiment a conjugate of PTH is provided wherein the PTH peptide of any of SEQ ID Nos: 7, 31-37 is modified by an acylated lysine substitution for the native amino acid at position 33 and/or for the native amino acid at the C-terminal amino acid of the PTH peptide, or the corresponding position of any analogs thereof, and a self-cleaving dipeptide is covalently linked via an amide bond to the N-terminal amino acid of the PTH peptide, optionally wherein the first amino acid of said self-cleaving dipeptide is acylated, optionally wherein the acylated amino acid comprises a C14-C20 fatty acid a C14-C20 diacid, a C16-C18 fatty acid or C16-C18 diacid.

In accordance with the present disclosure, the self-cleaving dipeptide comprises a combination of two amino acids that are linked to a primary amine of a PTH peptide such that under physiological conditions, the dipeptide will be spontaneously cleaved via a non-enzymatic degradation mechanism releasing the dipeptide from the PTH peptide. In one embodiment, one of the amino acids of the self-cleaving dipeptide, optionally the first amino acid, is acylated with a C14-C20 fatty acid or a C14-C20 diacid to further inhibit the activity of PTH to which it is covalently linked. In one embodiment the self-cleaving dipeptide is linked to a primary amine located on the side chain of an amino acid at position 13, 16, 19, 22, or 26 of the PTH peptide (relative to SEQ ID NO: 7) or at the N-terminal primary amine. Thus, the presence of the self-cleaving dipeptide delays the ability of the conjugate form to interact with its target receptor until chemical cleavage releases the PTH in an active form. In accordance with the present disclosure, one embodiment of a PTH conjugate of the present disclosure is PTH(1-33), dK⁻¹(γE-(miniPEG)₂-γE-COC₁₆H₃₂CO₂H), N(Me)G⁰, K³³(γE-2×OEG-γE-diacidC18) the complete structure of which is: k(X)(N-Me)GSVSEIQLMHNLGKHLNSMERVEWLRKKLQDVHK(X)-OH (SEQ ID NO: 87),
wherein k is d-Lys;

X is γE-(miniPEG)₂-γE-COC₁₆H₃₂CO₂H;

γE is the l-isomer of gamma, glutamic acid;

(miniPEG)₂ is COCH₂OCH₂CH₂OCH₂CH₂NH;

COC₁₆H₃₂CO₂H is C18 diacid;

(N-Me)G is sarcosine;

(SEQ ID NO: 7)
SVSEIQLMHNLGKHLNSMERVEWLRKKLQDVHK
is PTH + K33;

K is l-Lys; and
—OH designates the C-terminal amino acid has a terminal carboxylic acid.

In certain embodiments the molecular weight of a PTH conjugate of the present invention is 5873.9 Dalton.

In some embodiments the present invention provides PTH conjugates wherein an alternative moiety is used for one or more miniPEG in the conjugate. Non-limiting examples include

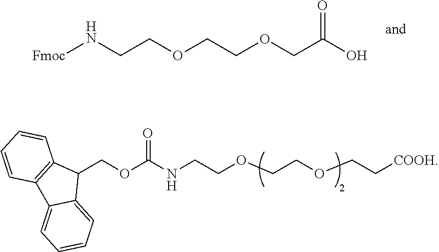

In some embodiments the present invention provides a PTH peptide wherein the PTH peptide comprises one or more of Tyr(OPO₃H₂):

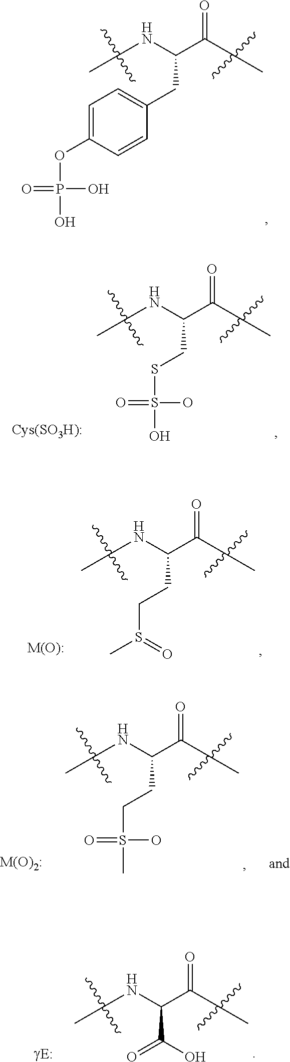

Advantageously, the rate of cleavage of the self-cleaving dipeptide depends on the structure and stereochemistry of the dipeptide element, and also on the strength of the nucleophile present on the dipeptide that induces cleavage to a diketopiperazine or diketomorpholine related entity. In one embodiment, based on the selected structure of the dipeptide, the non-enzymatic half time (t½) of the dipeptide/drug complex can be selected to be between 1-720 hrs under physiological conditions. Physiological conditions as disclosed herein are intended to include a temperature of about 35 to 40° C. and a pH of about 7.0 to about 7.4, and more typically include a pH of 7.2 to 7.4 and a temperature of 36 to 38° C. Since physiological pH and temperature are tightly regulated within a highly defined range, the speed of conversion from dipeptide/drug complex to drug will exhibit high intra and interpatient reproducibility. The speed of the chemical conversion will determine the time of onset and duration of in vivo biological action. Accordingly, the activation of the administered PTH analog of the present disclosure relies upon an intramolecular chemical reaction that is not dependent upon additional chemical additives, or enzymes, and wherein the speed of conversion is controlled by the inherent chemical nature of the dipeptide substituents.

In one embodiment, the self-cleaving dipeptide element is covalently bound to the PTH peptide via an amide linkage, and the dipeptide further comprises a depot polymer linked to an amino acid side chain of the self-cleaving dipeptide. In one embodiment two or more depot polymers are linked to a single self-cleaving dipeptide element. In one embodiment the depot polymer is selected to be biocompatible and of sufficient size that the PTH peptide with a covalently attached dipeptide remains sequestered at an injection site and/or incapable of interacting with its corresponding receptor upon administration to a patient. Subsequent cleavage of the dipeptide releases the PTH peptide to interact with its intended target. Selection of different combinations of substituents on the dipeptide element will allow for the preparation of injectable compositions that comprise a mixture of dipeptide/PTH peptides that release the drug over a desired time frame. Suitable depot polymers include but are not limited to dextrans, polylactides, polyglycolides, caprolactone-based polymers, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), polyvinylpyrrolidone, polyethylene glycol, polyhydroxycellulose, polysaccharides, chitin, chitosan, hyaluronic acid, and copolymers, terpolymers and mixtures thereof, and biodegradable polymers and their copolymers including caprolactone-based polymers, polycaprolactones and copolymers which include polybutylene terephthalate. In one embodiment the depot polymer is selected from the group consisting of polyethylene glycol, dextran, polylactic acid, polyglycolic acid and a copolymer of lactic acid and glycolic acid, and in one specific embodiment the depot polymer is polyethylene glycol. In one embodiment the depot polymer comprises one or more polyethylene glycol chains linked to the self-cleaving dipeptide element wherein the combined molecular weight of depot polymer(s) is 40,000 to 80,000 Daltons.

In accordance with one embodiment the self-cleaving dipeptide element is covalently bound to the PTH peptide via an amide linkage at an active site of the PTH to form a prodrug derivative of the drug. In one embodiment the first amino acid of the self-cleaving dipeptide is in the D-stereochemical configuration. In one embodiment the side chain of the first amino acid of the self-cleaving dipeptide is covalently linked to a moiety that enhances the retention of the PTH analog in a patient's bloodstream. In one embodiment the retention enhancing moiety linked to the first amino acid of the self-cleaving dipeptide comprises an alkyl chain of sufficient size to bind serum albumin upon administration to a patient. In one embodiment the side chain of the first amino acid of the self-cleaving dipeptide is covalently linked to an alkyl chain comprising 14-30, 14-22, 16-20, 16, 18, 20 or 22 carbon atoms. In one embodiment the first amino acid of the self-cleaving dipeptide is an alkylated or acylated amino acid in the D-stereochemical configuration. In one embodiment the first amino acid of the self-cleaving dipeptide is covalently linked to a fatty acid or fatty diacid having a length of 14-30, 14-22, 16-20, 16, 18, 20 or 22 carbon atoms.

In one embodiment a conjugate of a PTH peptide is provided wherein the PTH peptide is covalently linked to a self-cleaving dipeptide. In one embodiment the conjugate comprises the general structure of A-B-Q wherein
A is an amino acid or a hydroxyl acid, optionally wherein the side chain of the amino acid or a hydroxyl acid is covalently linked to depot polymer or an alkyl or acyl chain;
B is an N-alkylated amino acid;
Q is an acylated or alkylated PTH peptide in accordance with the present disclosure, with the proviso that when A is a non-acylated amino acid, then A is an amino acid in the D-stereochemical configuration. In a further embodiment, for any of the PTH conjugates disclosed herein, one of A or B of said A-B dipeptide can be a non-coded amino acid, including for example an amino acid in the D-stereochemical configuration.

In accordance with one embodiment the self-cleaving dipeptide element (A-B) comprises the structure:

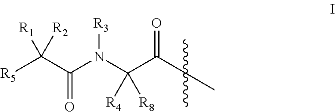

wherein
R₁, comprises a side chain selected from the group consisting of H, C₁-C₁₈ alkyl, C₂-C₁₈ alkenyl, (C₁-C₈ alkyl)OH, (C₁-C₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₅-C₆ cycloalkyl),

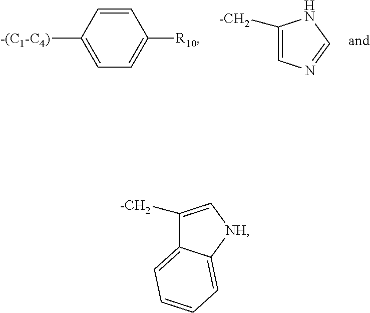

optionally further comprising a C16-C30 carbon chain covalently linked to said side chain;
R₂, and R₈ are independently H or C₁-C₆ alkyl;
R₄ is selected from the group consisting of H, C₁-C₁₈ alkyl, C₂-C₁₈ alkenyl, (C₁-C₈ alkyl)OH, (C₁-C₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₅-C₆ cycloalkyl),

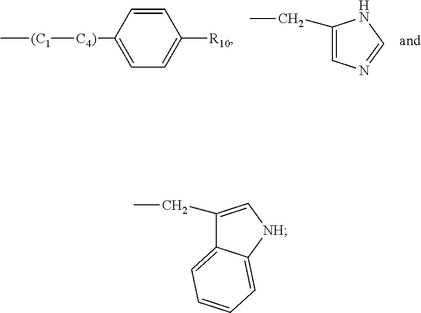

R₃ is selected from the group consisting of C₁-C₈ alkyl, or R₄ and R₃ together with the atoms to which they are attached form a pyrrolidine or piperidine ring;
R₅ is NH₂ or OH; and
R₁₀ is H, OH or NH₂, with the proviso that when R₄ and R₃ together with the atoms to which they are attached form a pyrrolidine ring, then R₁ and R₂ are not H.

In accordance with one embodiment the self-cleaving dipeptide element (A-B) comprises the structure:

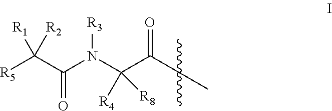

wherein
R₁, comprises a side chain selected from the group consisting of C₁-C₁₈ alkyl, (C₁-C₈ alkyl)OH, (C₁-C₈ alkyl)SH, and (C₁-C₄ alkyl)COOH, (C₁-C₄ alkyl)NH₂, optionally further comprising a C16-C30 fatty acid or C16-C30 diacid covalently linked to said side chain;
R₂, and R₈ are independently H or C₁-C₆ alkyl;
R₄ is selected from the group consisting of H, C₁-C₁₈ alkyl, C₂-C₁₈ alkenyl, (C₁-C₈ alkyl)OH, (C₁-C₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₅-C₆ cycloalkyl),

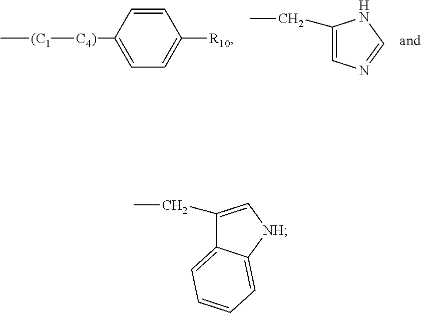

R₃ is selected from the group consisting of C₁-C₈ alkyl;
R₅ is NH₂; and
R₁₀ is H, OH or NH₂.

In accordance with one embodiment the self-cleaving dipeptide element (A-B) comprises the structure:

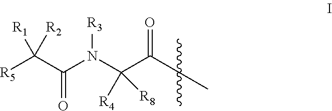

wherein
R₁, comprises a side chain selected from the group consisting of C₁-C₁₈ alkyl, (C₁-C₈ alkyl)OH, (C₁-C₈ alkyl)SH, (C₁-C₄ alkyl)COOH, (C₁-C₄ alkyl)NH₂, and a C16-C30 fatty acid or C16-C30 diacid covalently linked to said side chain, optionally via a spacer;
R₂, and R₈ are each H;
R₄ is selected from the group consisting of H and C₁-C₈ alkyl;
R₃ is C₁-C₆ alkyl; and
R₅ is NH₂, wherein said spacer is selected from the group consisting of a gamma glutamic acid, a gamma glutamic acid-gamma glutamic acid dipeptide, and a gamma glutamic acid-[COCH₂(OCH₂CH₂)ₖ—NH]_{q}-gamma glutamic acid, wherein k is an integer selected from the range of 1-8 or 2-4 and q is an integer selected from the range of 1-4. In one embodiment k is 2 or 4 and q is 1 or 2.

In accordance with one embodiment the self-cleaving dipeptide element (A-B) comprises the structure:

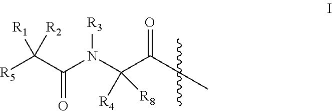

wherein
R₁ comprises a side chain of (C₁-C₄ alkyl)NH₂, optionally wherein a C16-C30 fatty acid or C16-C30 diacid is covalently linked to said side chain, optionally via a spacer;
R₂, R₄ and R₈ are independently H, or C₁-C₄ alkyl;
R₃ is C₁-C₆ alkyl; and
R₅ is NH₂ wherein said spacer is selected from the group consisting of a gamma glutamic acid, a gamma glutamic acid-gamma glutamic acid dipeptide, and a gamma glutamic acid-[COCH₂(OCH₂CH₂)ₖ—NH]_{q}-gamma glutamic acid, wherein k is an integer selected from the range of 1-8 or 2-4 and q is an integer selected from the range of 1-4, optionally wherein when R₁ consists of (C₁-C₄ alkyl)NH₂, then the first amino acid of the self-cleaving dipeptide is in the D-stereochemical configuration. In one embodiment k is 2 or 4 and q is 1 or 2.

In accordance with one embodiment the self-cleaving dipeptide element (A-B) comprises the structure:

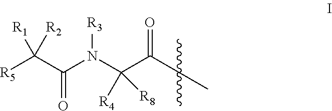

wherein
R₁, is selected from the group consisting of (C₁-C₈ alkyl)-CO(CH₂)₁₄₋₂₀CH₃, (C₁-C₈ alkyl)S—CO(CH₂)₁₄₋₂₀CH₃, (C₁-C₄ alkyl)NH—CO(CH₂)₁₄₋₂₀ CH₃, (C₁-C₈ alkyl)-CO(CH₂)₁₄₋₂₀COOH (C₁-C₈ alkyl)S—CO(CH₂)₁₄₋₂₀COOH, and (C₁-C₄ alkyl)NH—CO(CH₂)₁₄₋₂₀COOH;
R₂, and R₈ are each H;
R₄ is selected from the group consisting of H, C₁-C₈ alkyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₁-C₄ alkyl)(C₅-C₆ cycloalkyl),

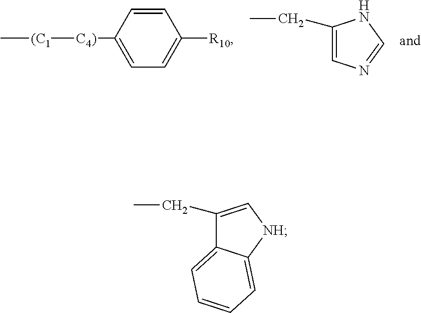

R₃ is selected from the group consisting of C₁-C₆ alkyl, or R₄ and R₃ together with the atoms to which they are attached form pyrrolidine or piperidine;
R₅ is NH₂; and
R₁₀ is H, OH or NH₂, with the proviso that when R₄ and R₃ together with the atoms to which they are attached form a pyrrolidine ring, then R₂ is not H, optionally wherein the chemical cleavage half-life (t_{1/2}) of A-B from said PTH peptide is at least about 20 to 240 hours in standard PBS solution under physiological conditions.

In accordance with one embodiment the self-cleaving dipeptide element (A-B) comprises the structure:

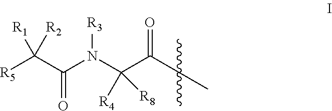

wherein
R₁, is selected from the group consisting of (C₁-C₄ alkyl)S—CO(CH₂)₁₄₋₂₀CH₃, (C₁-C₄ alkyl)NH—CO(CH₂)₁₄₋₂₀ CH₃, (C₁-C₄ alkyl)S—CO(CH₂)₁₄₋₂₀COOH, and (C₁-C₄ alkyl)NH—CO(CH₂)₁₄₋₂₀COOH;
R₂, and R₈ are each H;
R₄ is selected from the group consisting of H and C₁-C₈ alkyl;
R₃ is selected from the group consisting of C₁-C₆ alkyl; and
R₅ is NH₂, optionally wherein the chemical cleavage half-life (t_{1/2}) of A-B from said PTH peptide is at least about 48 to 168 hours in standard PBS solution under physiological conditions. In one further embodiment R₁, is (C₁-C₄ alkyl)NH—CO(CH₂)₁₄₋₂₀ CH₃, or (C₁-C₄ alkyl)NH—CO(CH₂)₁₄₋₂₀COOH; R₂, and R₈ are each H; R₄ is H or CH₃; R₃ is C₁-C₆ alkyl; and R₅ is NH₂.

In accordance with one embodiment the self-cleaving dipeptide element (A-B) comprises the structure:

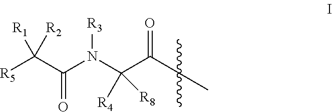

wherein
R₁, is selected from the group consisting of (C₁-C₄ alkyl)NH—CO(CH₂)₁₄₋₂₀CH₃, (C₁-C₄ alkyl)NH—CO(CH₂)₁₄₋₂₀COOH, (C₁-C₄ alkyl)NH-[spacer]-CO(CH₂)₁₄₋₂₀CH₃ and (C₁-C₄ alkyl)NH-[spacer]-CO(CH₂)₁₄₋₂₀COOH;
R₂, and R₈ are each H or C₁-C₄ alkyl;
R₄ and R₃ together with the atoms to which they are attached form a piperidine ring; and
R₅ is NH₂, optionally wherein the chemical cleavage half-life (t_{1/2}) of A-B from said PTH peptide is at least about 72 to 144 hours in standard PBS solution under physiological conditions, wherein said spacer comprises one or more moieties selected from a gamma glutamic acid and —[COCH₂(OCH₂CH₂)ₖ—NH]_{q}, optionally wherein said spacer is selected from the group consisting of a gamma glutamic acid, a gamma glutamic acid-gamma glutamic acid dipeptide, and a gamma glutamic acid-[COCH₂(OCH₂CH₂)ₖ—NH]_{q}-gamma glutamic acid, wherein k is an integer selected from the range of 1-8 or 2-4 and q is an integer selected from the range of 1-4, optionally wherein k is 2 or 4 and q is 1 or 2. In one further embodiment R₁, is (C₁-C₄ alkyl)NH—CO(CH₂)₁₄₋₂₀CH₃, (C₁-C₄ alkyl)NH—CO(CH₂)₁₄₋₂₀COOH, (C₁-C₄ alkyl)NH-[spacer]-CO(CH₂)₁₄₋₂₀CH₃ or (C₁-C₄ alkyl)NH-[spacer]-CO(CH₂)₁₄₋₂₀COOH; R₂, and R₈ are each H; R₄ is H or CH₃; R₃ is C₁-C₆ alkyl; and R₅ is NH₂.

In accordance with one embodiment the self-cleaving dipeptide element (A-B) comprises the structure:

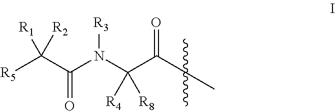

wherein
R₁, is (C₁-C₄ alkyl)NH—CO(CH₂)₁₆COOH or (C₁-C₄ alkyl)NH—CO(CH₂)₁₈COOH;
R₂ and R₈ are each H;
R₄ is H, or CH₃;
R₃ is CH₃ and
R₅ is NH₂, optionally wherein R₁, is (C₃-C₄ alkyl)NH—CO(CH₂)₁₆COOH and R₄ is CH₃.

In accordance with one embodiment the self-cleaving dipeptide element (A-B) comprises the structure:

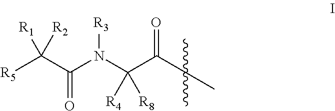

wherein
R₁, is (C₁-C₄ alkyl)NH-[spacer]-CO(CH₂)₁₆COOH or (C₁-C₄ alkyl)NH-[spacer]-CO(CH₂)₁₈COOH;
R₂ and R₈ are each H;
R₄ is H, or CH₃;
R₃ is CH₃ and
R₅ is NH₂, optionally wherein R₁, is (C₃-C₄ alkyl)NH-[spacer]-CO(CH₂)₁₆COOH and R₄ is CH₃, wherein said spacer is gamma glutamic acid-[COCH₂(OCH₂CH₂)ₖ—NH]_{q}-gamma glutamic acid, wherein k is an integer selected from the range of 1-8 or 2-4 and q is an integer selected from the range of 1-4, optionally wherein k is 2 or 4 and q is 1 or 2, optionally wherein k and q are both 2.

In one embodiment the PTH peptide of the PTH conjugates of the present disclosure comprises an amino acid sequence selected from the group consisting of

(SEQ ID NO: 20)
SVSEIQLMHNLGKHLNSMERVEWLRKKLQDVHNFVALGAPLA
PRDAGSQRPRKKEDNVLVESHEKSLGEADKADVNVLTKAKSQX₃₅;
(SEQ ID NO: 96)
AVSEHQLLHDKGKSIQDLRRRFFLHHLIAEIHTAEIRAX₃₅;
(SEQ ID NO: 21)
SVSEIQLMHNLGKHLNSMERVEWLRKKLQDVHNFVALX₃₅;
(SEQ ID NO: 12)
SVSEIQLMHNLGKHLNSMERVEWLRKKLQDVHNFX₃₅;
(SEQ ID NO: 14)
SVSEIQLMHNLGKHLNSMERVEWLRKKLQDVHQFX₃₅;
(SEQ ID NO: 4)
SVSEIQLMHNLX₁₂X₁₃HLX₁₆X₁₇MERVEWLRX₂₆X₂₇LQDX₃₁H-Z;
(SEQ ID NO: 5)
SVSEIQLMHNLGX₁₃HLNSMERVEWLRX₂₆X₂₇LQDX₃₁H-Z;
(SEQ ID NO: 6)
SVSEIQLMHNLX₁₂KHLX₅₆X₁₇MERVEWLRKKLQDVH-Z;
(SEQ ID NO: 133)
SVSEIQLMHX₁₀LGX₁₃HLX₁₆SX₁₈ERVEWLRX₂₆X₂₇LQDX₃₁H-Z,;
(SEQ ID NO: 134)
SVSEIQLMHX₁₀LX₁₂KHLX₁₆X₁₇X₁₈ERVEWLRKKLQDVH-Z;;
(SEQ ID NO: 135)
SVSEIQLMHX₁₀LGKHLX₁₆SX₁₈ERVEWLRKKLQDVH-Z;
(SEQ ID NO: 22)
SVSEIQLMHNLGKHLNSMERVEWLRKKLQDGH-Z;
(SEQ ID NO: 7)
SVSEIQLMHNLGKHLNSMERVEWLRKKLQDVH-Z;
(SEQ ID NO: 23)
SVSEIQLMHNLGEHLNSMERVEWLRKKLQDVH-Z;
(SEQ ID NO: 24)
SVSEIQLMHNLGKHLNSMERVEWLREKLQDVH-Z;
or
(SEQ ID NO: 25)
SVSEIQLMHNLGKHLNSMERVEWLRKELQDVH-Z;

wherein
Z is X₃₃, X₃₃F, X₃₃FX₃₅, X₃₃FVX₃₅ (SEQ ID NO: 136), X₃₃FVAX₃₅ (SEQ ID NO: 137), X₃₃FVALX₃₅ (SEQ ID NO: 138), X₃₃FVALGX₃₅ (SEQ ID NO: 139), X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally wherein Z is X₃₃, X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally Z is X₃₃F, X₅₃FX₃₅, or X₃₃;
X₁₀ and X₁₆ are independently Asp, Gln or Asn;
X₁₂ is amino isobutyric acid (Aib) or Gly;
X₅₆ is amino isobutyric acid (Aib) or Asn;
X₁₇ is amino isobutyric acid (Aib) or Ser;
X₁₈ is Met, Met(O), or Nleu;
X₁₃, X₂₆, and X₂₇ are independently selected from the group consisting of Arg, Glu, Asp and Lys;
X₃₁ is Gly or Val;
X₃₃ and X₃₅ each comprise an acylated or alkylated amino acid;
X₅₃ is Gln, Asp, Glu, or Asn, optionally wherein X₅₃ is Gln or Asn or X₅₃ is Asn, and optionally with the proviso that only one of X₁₂, X₁₆ and X₁₇ is Aib, optionally wherein the C-terminal amino acid is modified to replace the carboxy terminus with an amide.

In one embodiment the PTH peptide of the PTH conjugates of the present disclosure comprises an amino acid sequence selected from the group consisting of

(SEQ ID NO: 12)
SVSEIQLMHNLGKHLNSMERVEWLRKKLQDVHNFX₃₅;
(SEQ ID NO: 14)
SVSEIQLMHNLGKHLNSMERVEWLRKKLQDVHQFX₃₅;
(SEQ ID NO: 5)
SVSEIQLMHNLGX₁₃HLNSMERVEWLRX₂₆X₂₇LQDX₃₁H-Z;
(SEQ ID NO: 135)
SVSEIQLMHX₁₀LGKHLX₁₆SX₁₈ERVEWLRKKLQDVH-Z;
(SEQ ID NO: 22)
SVSEIQLMHNLGKHLNSMERVEWLRKKLQDGH-Z;
or
(SEQ ID NO: 7)
SVSEIQLMHNLGKHLNSMERVEWLRKKLQDVH-Z;

wherein
Z is X₃₃F, X₅₃FX₃₅, or X₃₃;
X₁₀ and X₁₆ are independently Asp, Gln or Asn;
X₁₈ is Met, Met(O), or Nleu;
X₁₃, X₂₆, and X₂₇ are independently selected from the group consisting of Arg, Glu, Asp and Lys;
X₃₁ is Gly or Val;
X₃₃ and X₃₅ each comprise an acylated or alkylated amino acid optionally wherein X₃₃ is an acylated Lys and X₃₅ is an acylated Cys;
X₅₃ is Gln, Asp, Glu, or Asn.

In one embodiment the PTH peptide of the PTH conjugates of the present disclosure comprises an amino acid sequence selected from the group consisting of

(SEQ ID NO: 5)
SVSEIQLMHNLGX₁₃HLNSMERVEWLRX₂₆X₂₇LQDX₃₁H-Z;
(SEQ ID NO: 22)
SVSEIQLMHNLGKHLNSMERVEWLRKKLQDGH-Z;
or
(SEQ ID NO: 7)
SVSEIQLMHNLGKHLNSMERVEWLRKKLQDVH-Z,

wherein
Z is X₃₃F, X₅₃FX₃₅, or X₃₃;
X₁₃, X₂₆, and X₂₇ are independently selected from the group consisting of Arg, Glu, Asp and Lys;
X₃₁ is Gly or Val;
X₃₃ and X₃₅ each independently is an amino acid having a side chain of (C₁-C₄ alkyl)NH—CO(CH₂)₁₄₋₂₀COOH, (C₁-C₄ alkyl)NH—CO(CH₂)₁₄₋₂₀CH₃, (C₁-C₄ alkyl)S—CO(CH₂)₁₄₋₂₀COOH, (C₁-C₄ alkyl)S—CO(CH₂)₁₄₋₂₀CH₃, (C₁-C₄ alkyl)NH-[spacer]-CO(CH₂)₁₄₋₂₀COOH, (C₁-C₄ alkyl)NH-[spacer]-CO(CH₂)₁₄₋₂₀CH₃, (C₁-C₄ alkyl)S-[spacer]-CO(CH₂)₁₄₋₂₀COOH, or (C₁-C₄ alkyl)S-[spacer]-CO(CH₂)₁₄₋₂₀CH₃; and
X₅₃ is Gln, Asp, Glu, or Asn, optionally wherein X₃₃ and X₃₅ are an amino acid having a side chain selected from the group consisting of (C₁-C₄ alkyl)NH—CO(CH₂)₁₄₋₂₀COOH, (C₁-C₄ alkyl)NH—CO(CH₂)₁₄₋₂₀CH₃, (C₁-C₄ alkyl)NH-[spacer]-CO(CH₂)₁₄₋₂₀COOH and (C₁-C₄ alkyl)NH-[spacer]-CO(CH₂)₁₄₋₂₀CH₃, wherein the spacer is selected from the group consisting of a gamma glutamic acid, a gamma glutamic acid-gamma glutamic acid dipeptide, and a gamma glutamic acid-[COCH₂(OCH₂CH₂)ₖ—NH]_{q}-gamma glutamic acid, wherein k is an integer selected from the range of 1-8 or 2-4 and q is an integer selected from the range of 1-4. In one embodiment k is 2 or 4 and q is 1 or 2.

In one embodiment a PTH peptide conjugate exhibiting an improved therapeutic index and improved in vivo retention time is provided, wherein the conjugate comprises an amino acid sequence selected from the group consisting of

(SEQ ID NO: 20)
SVSEIQLMHNLGKHLNSMERVEWLRKKLQDVHNFVALGAPLAPRDAG
SQRPRKKEDNVLVESHEKSLGEADKADVNVLTKAKSQX₃₅;
(SEQ ID NO: 21)
SVSEIQLMHNLGKHLNSMERVEWLRKKLQDVHNFVAL_{X35};
(SEQ ID NO: 12)
SVSEIQLMHNLGKHLNSMERVEWLRKKLQDVHNFX₃₅;
(SEQ ID NO: 14)
SVSEIQLMHNLGKHLNSMERVEWLRKKLQDVHQFX₃₅;
(SEQ ID NO: 4)
SVSEIQLMHNLX₁₂X₁₃HLX₁₆X₁₇MERVEWLRX₂₆X₂₇LQDX₃₁H-Z;
(SEQ ID NO: 5)
SVSEIQLMHNLGX₁₃HLNSMERVEWLRX₂₆X₂₇LQDX₃₁H-Z;
(SEQ ID NO: 6)
SVSEIQLMHNLX₁₂KHLX₅₆X₁₇MERVEWLRKKLQDVH-Z;
(SEQ ID NO: 135)
SVSEIQLMHX₁₀LGKHLX₁₆SX₁₈ERVEWLRKKLQDVH-Z;
(SEQ ID NO: 22)
SVSEIQLMHNLGKHLNSMERVEWLRKKLQDGH-Z;
(SEQ ID NO: 7)
SVSEIQLMHNLGKHLNSMERVEWLRKKLQDVH-Z;
(SEQ ID NO: 23)
SVSEIQLMHNLGEHLNSMERVEWLRKKLQDVH-Z;
(SEQ ID NO: 24)
SVSEIQLMHNLGKHLNSMERVEWLREKLQDVH-Z;
or
(SEQ ID NO: 25)
SVSEIQLMHNLGKHLNSMERVEWLRKELQDVH-Z;

wherein
Z is X₃₃, X₃₃F, X₃₃FX₃₅, X₃₃FVX₃₅ (SEQ ID NO: 136), X₃₃FVAX₃₅ (SEQ ID NO: 137), X₃₃FVALX₃₅ (SEQ ID NO: 138), X₃₃FVALGX₃₅ (SEQ ID NO: 139), X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally Z is X₃₃, X₅₃X₃₅, X₅₃FX₃₅, X₅₃FVX₃₅, X₅₃FVAX₃₅, X₅₃FVALX₃₅, or X₅₃FVALGX₃₅, optionally Z is X₃₃F, X₅₃FX₃₅, or X₃₃;
X₁₀ and X₁₆ are independently Asp, Gln or Asn;
X₁₂ is amino isobutyric acid (Aib) or Gly;
X₅₆ is amino isobutyric acid (Aib) or Asn;
X₁₇ is amino isobutyric acid (Aib) or Ser;
X₁₈ is Met, Met(O), or Nleu;
X₁₃, X₂₆, and X₂₇ are independently selected from the group consisting of Arg, Glu, Asp and Lys;
X₃₁ is Gly or Val;
X₃₃ and X₃₅ each comprise an acylated or alkylated amino acid;
X₅₃ is Gln, Asp, Glu, or Asn, optionally with the proviso that only one of X₁₂, X₁₆ and X₁₇ is Aib; and
said self-cleaving dipeptide comprising the general structure

A-B—;

wherein

A is an acylated or alkylated amino acid;

B is an N-alkylated amino acid;

wherein said acylated or alkylated amino acid of each of X₃₃, X₃₅ and A is an amino acid comprising a C16-C30 carbon chain covalently linked to its amino acid side chain, optionally via a spacer, and said self-cleaving dipeptide is linked to said PTH peptide through formation of an amide bond between B and the N-terminal alpha amine of said PTH peptide. In one embodiment A and B are selected to provide a chemical cleavage half-life (t½) of A-B from said PTH peptide of at least about 24 hours to about 96 hours, or about 48 to about 96 hours, or about 72 to about 120 hours in standard PBS solution under physiological conditions.

In one embodiment a conjugate is provided that comprises a PTH peptide/PTHrP and a self-cleaving dipeptide, wherein the dipeptide is covalently bound to said PTH/PTHr peptide via an amide bond, optionally at the N-terminal alpha amine of the PTH peptide, wherein the PTH/PTHr peptide comprises an amino acid sequence selected from the group consisting of

(SEQ ID NO: 39)
SRRLKRAVSEHQLLHDKGKSIQDLRRRFFLHHLIAEIHTAEIRATSEVS
PNSKPSPNTKNHPVRFGSDDEGRYLTQETNKVETYKEQPLKTPGKKKKG
KPGKRKEQEKKKRRTRSAWLDSGVTGSGLEGDHLSDTSTTSLELDSRR
H-X₃₃;
(SEQ ID NO: 5)
SVSEIQLMHNLGX₁₃HLNSMERVEWLRX₂₆X₂₇LQDX₃₁H-Z;
(SEQ ID NO: 6)
SVSEIQLMHNLX₁₂KHLX₅₆X₁₇MERVEWLRKKLQDVH-Z;
and
(SEQ ID NO: 7)
SVSEIQLMHNLGKHLNSMERVEWLRKKLQDVH-Z;

wherein
Z is X₃₃, X₃₃F, X₃₃FX₃₅, X₃₃FVX₃₅ (SEQ ID NO: 136), X₃₃FVAX₃₅ (SEQ ID NO: 137), X₃₃FVALX₃₅ (SEQ ID NO: 138), X₃₃FVALGX₃₅ (SEQ ID NO: 139), X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally wherein Z is X₃₃, X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143);
X₁₂ is amino isobutyric acid (Aib) or Gly;
X₅₆ is amino isobutyric acid (Aib) or Asn;
X₁₇ is amino isobutyric acid (Aib) or Ser;
X₁₃, X₂₆, and X₂₇ are independently selected from the group consisting of Arg, Glu, Asp and Lys;
X₃₁ is Gly or Val;
X₃₃ and X₃₅ each comprise an acylated amino acid, optionally wherein X₃₃ and X₃₅ are independently selected from an amino acid having the general structure of

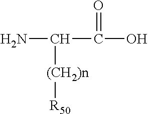

wherein n is an integer selected from the range of 1-4 and R₅₀ is NH₂, COOH or SH, optionally wherein the acylated amino acid of X₃₃ and X₃₅ is independently selected from lysine, ornithine, cysteine or homocysteine wherein the side chain of said acylated amino acid is covalently linked to a C16-C22 fatty acid or C16-C22 diacid, optionally through a spacer; and
X₅₃ is Gln or Asn, optionally with the proviso that only one of X₁₂, X₁₆ and X₁₇ is Aib; and
said self-cleaving dipeptide comprises the general structure

A-B—;

wherein

A is an amino acid or acylated amino acid optionally wherein said amino acid is selected from a cysteine or lysine, wherein the side chain of said cysteine or lysine is optionally covalently linked to a C16-C22 fatty acid or C16-C22 diacid, optionally via a spacer joining the C16-C22 fatty acid or C16-C22 diacid to the amino acid side chain;

B is an N-alkylated amino acid, optionally wherein B is N-methyl glycine or N-methyl alanine;

In one embodiment, the acylated amino acid of each of X₃₃, X₃₅ and A is an amino acid comprising a C16-C30 fatty acid or C16-C30 diacid covalently linked to its amino acid side chain, optionally via a spacer and said self-cleaving dipeptide is linked to said PTH peptide through formation of an amide bond between B and the N-terminal alpha amine of said PTH peptide. Optionally A is in the D-stereochemical configuration. In one embodiment a conjugate is provided that comprises a PTH peptide and a self-cleaving dipeptide, wherein the dipeptide is covalently bound to said PTH peptide via an amide bond, optionally at the N-terminal alpha amine of the PTH peptide, wherein

the PTH peptide comprises an amino acid sequence selected from the group consisting of

(SEQ ID NO: 5)
SVSEIQLMHNLGX₁₃HLNSMERVEWLRX₂₆X₂₇LQDX₃₁H-Z;
(SEQ ID NO: 6)
SVSEIQLMHNLX₁₂KHLX₅₆X₁₇MERVEWLRKKLQDVH-Z;
(SEQ ID NO: 135)
SVSEIQLMHX₁₀LGKHLX₁₆SX₁₈ERVEWLRKKLQDVH-Z;
and
(SEQ ID NO: 7)
SVSEIQLMHNLGKHLNSMERVEWLRKKLQDVH-Z;

wherein
Z is X₃₃, X₃₃F, X₅₃FX₃₅, or;
X₁₀ and X₁₆ are independently Asp, Gln or Asn;
X₁₂ is amino isobutyric acid (Aib) or Gly;
X₅₆ is amino isobutyric acid (Aib) or Asn;
X₁₇ is amino isobutyric acid (Aib) or Ser;
X₁₈ is Met, Met(O), or Nleu;
X₁₃, X₂₆, and X₂₇ are independently selected from the group consisting of Arg, Glu, Asp and Lys;
X₃₁ is Gly or Val;
X₃₃ and X₃₅ each comprise an acylated amino acid, optionally wherein X₃₃ and X₃₅ are independently selected from cysteine, homocysteine, ornithine or lysine, wherein the side chain of said cysteine, homocysteine, ornithine or lysine is covalently linked to a C16-C22 fatty acid or C16-C22 diacid, optionally through a spacer;
X₅₃ is Gln or Asn, optionally with the proviso that only one of X₁₂, X₁₆ and X₁₇ is Aib; and
said self-cleaving dipeptide comprises the general structure

A-B—;

wherein

A is an acylated amino acid optionally selected from a cysteine or lysine wherein the side chain of said cysteine or lysine is covalently linked to a C16-C22 fatty acid or C16-C22 diacid, optionally through a spacer;

B is an N-alkylated amino acid, optionally wherein B is N-methyl glycine or N-methyl alanine;

wherein said acylated amino acid of each of X₃₃, X₃₅ and A is an amino acid comprising a C16-C30 fatty acid or C16-C30 diacid covalently linked to its amino acid side chain, optionally via a spacer and said self-cleaving dipeptide is linked to said PTH peptide through formation of an amide bond between B and the N-terminal alpha amine of said PTH peptide. Optionally A is in the D-stereochemical configuration.

In one embodiment a conjugate is provided comprising a PTH peptide and a self-cleaving dipeptide, wherein the dipeptide is covalently bound to said PTH peptide via an amide bond, optionally at the N-terminal alpha amine of the PTH peptide, wherein
the PTH peptide comprises the sequence of SVSEIQLMHNLGKHLNSMERVEWLRKKLQDVH-Z (SEQ ID NO: 7), or a peptide that differs from SEQ ID NO: 7 by 1 or 2 amino acid substitutions,
   wherein
   Z is X₃₃F, NFX₃₅, or X₃₃;
   X₃₃ and X₃₅ are each independently an amino acid comprising a C16-C30 fatty acid or C16-C30 diacid covalently linked to the side chain of the amino acid, optionally via a spacer, optionally wherein X₃₃ and X₃₅ are independently selected from a cysteine or lysine wherein the side chain of said cysteine or lysine is covalently linked to a C16-C22 fatty acid or C16-C22 diacid, optionally through a spacer; and
   X₅₃ is Gln or Asn, and the self-cleaving dipeptide comprises the structure:

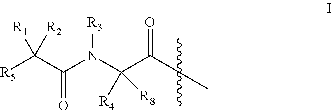

wherein
R₁, comprises a side chain selected from the group consisting of C₁-C₈ alkyl, (C₁-C₄ alkyl)OH, (C₁-C₄ alkyl)SH, (C₁-C₄ alkyl)COOH, and (C₁-C₄ alkyl)NH₂, and optionally a C16-C30 carbon chain, wherein said C16-C30 carbon chain, when present is covalently linked to said side chain, optionally via a spacer;
R₂, R₄ and R₈ are independently H, or C₁-C₄ alkyl;
R₃ is C₁-C₆ alkyl or R₃ and R₄ together with the atoms to which they are attached form a piperidine ring; and
R₅ is NH₂.

In one embodiment a conjugate is provided comprising a PTH peptide and any of the self-cleaving dipeptides disclosed herein, wherein the dipeptide is covalently bound to said PTH peptide via an amide bond, optionally at the N-terminal alpha amine of the PTH peptide, wherein
the PTH peptide comprises the sequence of SVSEIQLMHNLGKHLNSMERVEWLRKKLQDVH-Z (SEQ ID NO: 7), or a peptide that differs from SEQ ID NO: 7 by 1, 2 or 3 amino acid substitutions,
   wherein
   Z is X₃₃, X₃₃F, X₃₃FX₃₅, X₃₃FVX₃₅ (SEQ ID NO: 136), X₃₃FVAX₃₅ (SEQ ID NO: 137), X₃₃FVALX₃₅ (SEQ ID NO: 138), X₃₃FVALGX₃₅ (SEQ ID NO: 139), X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally wherein Z is X₃₃, X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143); optionally wherein Z is X₃₃, X₅₃X₃₅, or X₅₃FX₃₅;
   X₃₃ and X₃₅ are each independently an amino acid comprising a C16-C30 carbon chain covalently linked to the side chain of the amino acid, optionally via a spacer, optionally wherein X₃₃ and X₃₅ are independently selected from a cysteine or lysine wherein the side chain of said cysteine or lysine is covalently linked to a C16-C22 fatty acid or C16-C22 diacid, optionally through a spacer; and
   X₅₃ is Gln or Asn. In one embodiment the self-cleaving dipeptide comprises the structure:

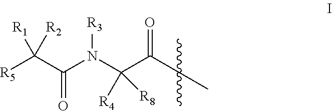

wherein
R₁, comprises a side chain selected from the group consisting of (C₁-C₄ alkyl)NH₂, and optionally a C16-C30 carbon chain, wherein said C16-C30 carbon chain, when present, is covalently linked to said side chain, optionally via a spacer;
R₂, R₄ and R₈ are independently H, or C₁-C₄ alkyl;
R₃ is C₁-C₆ alkyl or R₃ and R₄ together with the atoms to which they are attached form a piperidine ring; and
R₅ is NH₂.

In one embodiment a conjugate is provided comprising a PTH peptide and a self-cleaving dipeptide, wherein the dipeptide is covalently bound to said PTH peptide via an amide bond, optionally at the N-terminal alpha amine of the PTH peptide, wherein
the PTH peptide comprises the sequence of SVSEIQLMHNLGKHLNSMERVEWLRKKLQDVH-Z (SEQ ID NO: 7), or a peptide that differs from SEQ ID NO: 7 by 1, 2 or 3 amino acid substitutions,
   wherein
   Z is X₃₃, X₃₃F, X₃₃FX₃₅, X₃₃FVX₃₅ (SEQ ID NO: 136), X₃₃FVAX₃₅ (SEQ ID NO: 137), X₃₃FVALX₃₅ (SEQ ID NO: 138), X₃₃FVALGX₃₅ (SEQ ID NO: 139), X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally wherein Z is X₃₃, X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally wherein Z is X₃₃, X₅₃X₃₅, or X₅₃FX₃₅;
   X₃₃ and X₃₅ are each independently an amino acid comprising a side chain selected from the group consisting of (C₁-C₄ alkyl)NH-[spacer]-CO(CH₂)₁₄₋₂₀COOH, (C₁-C₄ alkyl)NH-[spacer]-CO(CH₂)₁₄₋₂₀CH₃, (C₁-C₄ alkyl)NH—CO(CH₂)₁₄₋₂₀COOH, or (C₁-C₄ alkyl)NH—CO(CH₂)₁₄₋₂₀CH₃;
   X₅₃ is Asn, and the self-cleaving dipeptide comprises the structure:

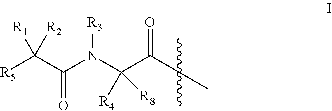

wherein
R₁, is selected from the group consisting of (C₁-C₄ alkyl)NH-[spacer]-CO(CH₂)₁₄₋₂₀COOH, (C₁-C₄ alkyl)NH-[spacer]-CO(CH₂)₁₄₋₂₀CH₃, (C₁-C₄ alkyl)NH—CO(CH₂)₁₄₋₂₀COOH, and (C₁-C₄ alkyl)NH—CO(CH₂)₁₄₋₂₀CH₃;
R₂, R₄ and R₈ are independently H, or C₁-C₄ alkyl;
R₃ is C₁-C₆ alkyl or R₃ and R₄ together with the atoms to which they are attached form a piperidine ring; and
R₅ is NH₂, optionally wherein the first amino acid is in the D-stereochemical configuration. In a further embodiment R₂, and R₈ are both H, and R₃ and R₄ are independently C₁-C₄ alkyl.

In one embodiment, a PTH conjugate is provided wherein the conjugate comprises a PTH peptide and a self-cleaving dipeptide, wherein the dipeptide is covalently bound to said PTH peptide via an amide bond at the N-terminal alpha amine of the PTH peptide, wherein the PTH peptide comprises the sequence of SVSEIQLMHNLGKHLNSMERVEWLRKKLQDVH-Z (SEQ ID NO: 7), wherein
Z is X₃₃, X₃₃F, X₃₃FV, NX₃₅, or NFX₃₅;
X₃₃ and X₃₅ are each independently an amino acid (optionally a lysine or ornithine) comprising a C16-C20 carbon chain covalently linked to the side chain of the amino acid, optionally via a spacer, optionally wherein X₃₃ and X₃₅ are independently a lysine acid comprising a C16-C20 fatty acid or C16-C20 diacid covalently linked to the lysine side chain, optionally wherein X₃₃ and X₃₅ are independently an amino acid comprising a side chain having the structure (C₁-C₄ alkyl)NH-[spacer]CO(CH₂)₁₄₋₂₀COOH;
X₅₃ is Gln or Asn, and the self-cleaving dipeptide comprises the structure:

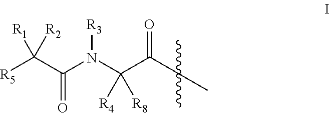

wherein

R₁, comprises a side chain of (C₁-C₄ alkyl)NH₂, and optionally a C16-C20 carbon chain, wherein said C16-C20 carbon chain, when present, is covalently linked to said side chain, optionally via a spacer, optionally wherein the side chain is acylated with a C16-C20 fatty acid or C16-C20 diacid, optionally via a spacer;

R₂, R₄ and R₈ are independently H, or C₁-C₄ alkyl;

R₃ is C₁-C₆ alkyl; and

R₅ is NH₂, optionally with the proviso that when R₁ lacks said C16-C20 carbon chain, the first amino acid of the dipeptide of Formula I is in the D-stereochemical configuration.

In one embodiment, a PTH conjugate is provided wherein the conjugate comprises a PTH peptide and a self-cleaving dipeptide, wherein the dipeptide is covalently bound to said PTH peptide via an amide bond at the N-terminal alpha amine of the PTH peptide, wherein the PTH peptide comprises the sequence of SVSEIQLMHNLGKHLNSMERVEWLRKKLQDVH-Z (SEQ ID NO: 7), wherein Z is X₃₅, NX₃₅, or NFX₃₅; wherein X₃₅ is an amino acid comprising a side chain having the structure (C₁-C₄ alkyl)NH-[spacer]CO(CH₂)₁₄₋₂₀COOH, optionally (C₄ alkyl)NH-[spacer]CO(CH₂)₁₄₋₂₀COOH, and said dipeptide comprises the structure A-B, where A is Lys, epsilon acylated Lys, epsilon acylated dLys or dLys and B is N-methyl glycine (sarcosine), optionally wherein the acylated Lys, or acylated dLys of A comprising a side chain having the structure (C₄ alkyl)NH-[spacer]CO(CH₂)₁₄₋₂₀COOH wherein said spacer is selected from the group consisting of gamma glutamic acid, a gamma glutamic acid dimer or a gamma glutamic acid-[COCH₂(OCH₂CH₂)ₖ—NH]_{q}-gamma glutamic acid, wherein k is an integer selected from the range of 1-8 or 2-4, and q is an integer selected from 1-4, optionally wherein k is 2 or 4 and q is 2, optionally wherein the spacer is gamma glutamic acid-[COCH₂(OCH₂CH₂)₂—NH—COCH₂(OCH₂CH₂)₂—NH-gamma glutamic acid having the structure of

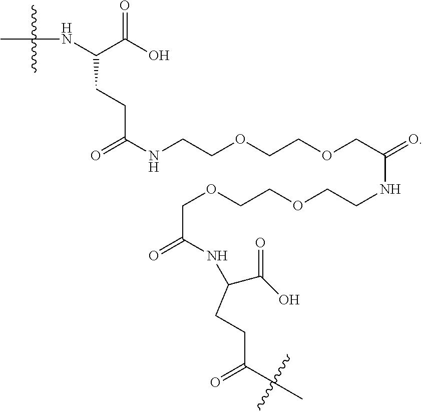

In one embodiment, a PTH conjugate is provided wherein the conjugate comprises a PTH peptide and a self-cleaving dipeptide, wherein the dipeptide is covalently bound to said PTH peptide via an amide bond at the N-terminal alpha amine of the PTH peptide, wherein the PTH peptide comprises the sequence of SVSEIQLMHNLGKHLNSMERVEWLRKKLQDVH-Z (SEQ ID NO: 7), wherein Z is X₃₅, NX₃₅, or NFX₃₅; wherein X₃₅ is an amino acid comprising a side chain having the structure (C₁-C₄ alkyl)NH-[spacer]CO(CH₂)₁₄₋₂₀COOH, optionally (C₄ alkyl)NH-[spacer]CO(CH₂)₁₄₋₂₀COOH, and said dipeptide comprises the structure A-B, where A is an amino acid in the L or D stereochemical configuration and comprising a side chain structure of (C₁-C₄ alkyl)NH-[spacer]CO(CH₂)₁₄₋₂₀COOH, optionally (C₄ alkyl)NH-[spacer]CO(CH₂)₁₄₋₂₀COOH, and B is N-methyl glycine (sarcosine), wherein said spacer is selected from the group consisting of gamma glutamic acid, a gamma glutamic acid dimer or a gamma glutamic acid-[COCH₂(OCH₂CH₂)ₖ—NH]_{q}-gamma glutamic acid, wherein k is an integer selected from the range of 1-8 or 2-4 and q is an integer selected from the range of 1-4, optionally wherein k is 2 or 4 and q is 2, optionally wherein the spacer is gamma glutamic acid-[COCH₂(OCH₂CH₂)₂NH]₂-gamma glutamic acid.

In accordance with any of the conjugate embodiments disclosed herein the spacer of the conjugates, when present, comprises an amino acid or dipeptide. In one embodiment the amino acid of the spacer is gamma glutamic acid. In one embodiment the spacer comprises two amino acids separated by a polyethylene glycol polymer. In accordance with any of the conjugate embodiments disclosed herein, the spacer may comprise the structure: gamma glutamic acid-[COCH₂(OCH₂CH₂)ₖ—NH]_{q}-gamma glutamic acid, wherein k is an integer selected from the range of 1-8 or 2-4 and q is an integer selected from the range of 1-4. In one embodiment k is 2 or 4 and q is 1 or 2. In one embodiment k is 2 and q is 2 or 4. In one embodiment k is 2 and q is 2. In one embodiment k is 2 and q is 4. In one embodiment k is 2 and q is 8. In one embodiment k is 2 or 4 and q is 1. In one embodiment k is 2 and q is 1. In one embodiment k is 4 and q is 1. In one embodiment k is 8 and q is 1. In one embodiment k is 1 and q is an integer selected from 2 to 8 or 2 to 4. In one embodiment k is 2 and q is an integer selected from 2 to 8 or 2 to 4. In one embodiment k is 2 and q is 1.

In one embodiment the PTH peptide of the conjugate comprises the sequence of SVSEIQLMHNLGKHLNSMERVEWLRKKLQDVHX₃₃ (SEQ ID NO: 2) or SVSEIQLMHNLGKHLNSMERVEWLRKKLQDVHNFX₃₅ (SEQ ID NO: 26),
wherein X₃₃ and X₃₅ are each independently an amino acid comprising a C16-C30 carbon chain covalently linked to the side chain of the amino acid, optionally via a spacer, optionally wherein X₃₃ and X₃₅ are each independently an amino acid comprising a side chain acylated with a C16-C20 fatty acid or C16-C20 diacid, optionally via a spacer and the self-cleaving dipeptide comprises the structure:

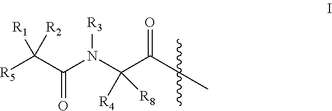

wherein
R₁ is (C₁-C₄ alkyl)NH—CO(CH₂)₁₄₋₂₀COOH, (C₁-C₄ alkyl)NH—CO(CH₂)₁₄₋₂₀CH₃, (C₁-C₄ alkyl)NH-[spacer]CO(CH₂)₁₄₋₂₀COOH or (C₁-C₄ alkyl)NH-[spacer]CO(CH₂)₁₄₋₂₀CH₃;
R₂, R₄ and R₈ are independently H, or C₁-C₄ alkyl;
R₃ is C₁-C₆ alkyl; and
R₅ is NH₂, optionally wherein R₂ and R₈ are both H, and R₄ is H or CH₃.

In one embodiment the PTH conjugate comprises a self-cleavable dipeptide covalently linked to the N-terminal alpha amine of a PTH peptide, wherein the PTH peptide comprises the sequence of

(SEQ ID NO: 2)
SVSEIQLMHNLGKHLNSMERVEWLRKKLQDVHX₃₃
or
(SEQ ID NO: 26)
SVSEIQLMHNLGKHLNSMERVEWLRKKLQDVHNFX₃₅,

wherein
X₃₃ and X₃₅ are each an amino acid comprising a side chain having a structure selected from the group consisting of (C₁-C₄ alkyl)NH—CO(CH₂)₁₄₋₂₀COOH, (C₁-C₄ alkyl)NH—CO(CH₂)₁₄₋₂₀CH₃, (C₁-C₄ alkyl)NH-[spacer]-CO(CH₂)₁₄₋₂₀COOH or (C₁-C₄ alkyl)NH-[spacer]-CO(CH₂)₁₄₋₂₀CH₃, wherein the spacer is selected from the group consisting of gamma glutamic acid, a gamma glutamic acid dimer or a gamma glutamic acid-[COCH₂(OCH₂CH₂)ₖ—NH]_{q}-gamma glutamic acid, wherein k is an integer selected from the range of 1-8 or 2-4, and q is an integer selected from the range of 1-4, optionally wherein k is 2 and q is 2;
and the self-cleaving dipeptide comprises the structure:

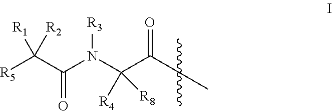

wherein
R₁ is a structure selected from the group consisting of (C₁-C₄ alkyl)NH—CO(CH₂)₁₄₋₂₀COOH, (C₁-C₄ alkyl)NH—CO(CH₂)₁₄₋₂₀CH₃, (C₁-C₄ alkyl)NH-[spacer]-CO(CH₂)₁₄₋₂₀COOH or (C₁-C₄ alkyl)NH-[spacer]-CO(CH₂)₁₄₋₂₀CH₃, wherein the spacer is selected from the group consisting of gamma glutamic acid, a gamma glutamic acid dimer or a gamma glutamic acid-[COCH₂(OCH₂CH₂)ₖ—NH]_{q}-gamma glutamic acid, wherein k is an integer selected from the range of 1-8 or 2-4, and q is an integer selected from the range of 1-4;
R₂, R₄ and R₈ are independently H, or C₁-C₄ alkyl;
R₃ is C₁-C₆ alkyl; and
R₅ is NH₂, optionally wherein R₂ and R₈ are both H, and R₄ is H or CH₃.

In one embodiment the PTH conjugate comprises a self-cleavable dipeptide covalently linked to the N-terminal alpha amine of a PTH peptide, wherein the PTH peptide comprises the sequence of

(SEQ ID NO: 2)
SVSEIQLMHNLGKHLNSMERVEWLRKKLQDVHX₃₃

wherein
X₃₃ is an amino acid comprising a side chain having a structure selected from the group consisting of (C₁-C₄ alkyl)NH-[spacer]-CO(CH₂)₁₄₋₂₀COOH or (C₁-C₄ alkyl)NH-[spacer]-CO(CH₂)₁₄₋₂₀CH₃, wherein the spacer is gamma glutamic acid-[COCH₂(OCH₂CH₂)ₖ—NH]_{q}-gamma glutamic acid, wherein k is an integer selected from the range of 2-4 and q is 1 or 2, optionally wherein k and q are both 2;
and the self-cleaving dipeptide comprises the structure:

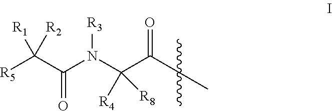

wherein
R₁ is a structure selected from the group consisting of (C₁-C₄ alkyl)NH-[spacer]-CO(CH₂)₁₄₋₂₀COOH or (C₁-C₄ alkyl)NH-[spacer]-CO(CH₂)₁₄₋₂₀CH₃, wherein the spacer is gamma glutamic acid-[COCH₂(OCH₂CH₂)ₖ—NH]_{q}-gamma glutamic acid, wherein k is an integer selected from the range of 2-4 and q is 1 or 2, optionally wherein k and q are both 2;
R₂, R₄ and R₈ are independently H, or C₁-C₄ alkyl;
R₃ is C₁-C₆ alkyl; and
R₅ is NH₂, optionally wherein R₂ and R₈ are both H, R₃ is C₁-C₃ alkyl. And R₄ is H or CH₃.

In one embodiment a conjugate is provided that comprises a PTH peptide and a self-cleaving dipeptide covalently bound to the N terminal alpha amine of the PTH peptide via an amide bond, wherein the PTH peptide comprises an amino acid sequence of

(SEQ ID NO: 7)
SVSEIQLMHNLGKHLNSMERVEWLRKKLQDVH-Z

wherein
Z is X₃₃F, X₅₃FX₃₅, or X₃₃;
X₃₃ and X₃₅ each independently comprise a cysteine, homocysteine, ornithine, d-lysine or lysine, wherein the side chain of the cysteine, homocysteine, ornithine, d-lysine or lysine residue is acylated or alkylated with a C14-C30 carbon chain;
X₅₃ is Gln or Asn; and
said self-cleaving dipeptide comprises the general structure:

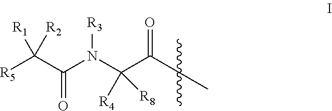

wherein

R₁, is a polymer chain comprising a side chain selected from the group consisting of (C₁-C₄ alkyl)SH, or (C₁-C₄ alkyl)NH₂, and a C16-C30 carbon chain covalently linked to said side chain;

R₂, R₄ and R₈ are independently H, or C₁-C₄ alkyl;

R₃ is C₁-C₆ alkyl; and

R₅ is NH₂, optionally wherein R₁, is (C₁-C₄ alkyl)NH—CO(CH₂)₁₄₋₂₀COOH; R₂ and R₈ are each H; R₄ is H; R₃ is CH₃ and R₅ is NH₂.

In accordance with one embodiment a conjugate is provided wherein the conjugate comprises a PTH peptide and a self-cleaving dipeptide, wherein the dipeptide is covalently linked to the N-terminal alpha amine of said PTH peptide via an amide bond, further wherein
the PTH peptide comprising the amino acid sequence of

(SEQ ID NO: 2)
SVSEIQLMHNLGKHLNSMERVEWLRKKLQDVHX₃₃,
or
(SEQ ID NO: 12)
SVSEIQLMHNLGKHLNSMERVEWLRKKLQDVHNFX₃₅,

wherein
X₃₃ and X₃₅ are each an amino acid comprising a side chain of (C₁-C₄ alkyl)NH-gamma glutamic acid-[COCH₂(OCH₂CH₂)ₖ—NH]_{q}-gamma glutamic acid-CO(CH₂)₁₄₋₂₀COOH; and
said self-cleaving dipeptide comprises the general structure:

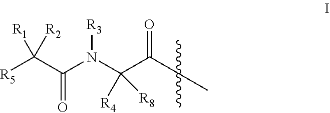

wherein
R₁, is (C₁-C₄ alkyl)NH-gamma glutamic acid-[COCH₂(OCH₂CH₂)ₖ—NH]_{q}-gamma glutamic acid-CO(CH₂)₁₄₋₂₀COOH;
R₂ and R₈ are each H;
R₄ is H, or CH₃;
R₃ is C₁-C₃ alkyl;
R₅ is NH₂;
k is and integer selected from 1, 2, 3, 4, 5, 6, 7 or 8; and
q is an integer selected from 1, 2, 3, 4, 5 and 6, optionally wherein R₄ is H and k is 2 or 4 and q is 1 or 2, optionally wherein K is 2 and q is 2.

In accordance with one embodiment a conjugate comprising a PTH peptide and a self-cleaving dipeptide is provided wherein the dipeptide is covalently linked to the N-terminal alpha amine of said PTH peptide via an amide bond, wherein the PTH peptide comprises the amino acid sequence of
SVSEIQLMHNLGKHLNSMERVEWLRKKLQDVHNFX₃₅ (SEQ ID NO: 12), wherein
X₃₅ is an amino acid comprising a side chain of (C₁-C₄ alkyl)NH-gamma glutamic acid-[COCH₂(OCH₂CH₂)ₖ—NH]_{q}-gamma glutamic acid-CO(CH₂)₁₄₋₂₀COOH; and
said self-cleaving dipeptide comprises the general structure:

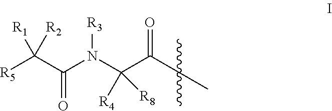

wherein
R₁, is (C₄ alkyl)NH-gamma glutamic acid-[COCH₂(OCH₂CH₂)ₖ—NH]_{q}-gamma glutamic acid-CO(CH₂)₁₄₋₂₀COOH;
R₂, R₄ and R₈ are each H;
R₃ is CH₃;
R₅ is NH₂;
k is an integer selected from the range of 1-4; and
q is an integer selected from the range of 1-4, optionally wherein k is 1 or 2 and q is 1, 2 or 4, optionally wherein k and q are both 2.

In accordance with one embodiment a conjugate comprising a PTH peptide and a self-cleaving dipeptide is provided wherein the dipeptide is covalently linked to the N-terminal alpha amine of said PTH peptide via an amide bond, wherein the PTH peptide comprises the amino acid sequence of

(SEQ ID NO: 2)
SVSEIQLMHNLGKHLNSMERVEWLRKKLQDVHX₃₃,

wherein
X₃₃ is an amino acid comprising a side chain of (C₁-C₄ alkyl)NH-gamma glutamic acid-[COCH₂(OCH₂CH₂)ₖ—NH]_{q}-gamma glutamic acid-CO(CH₂)₁₄₋₂₀COOH; and
said self-cleaving dipeptide comprises the general structure:

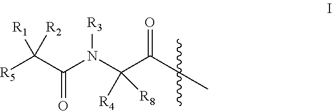

wherein
R₁, is (C₄ alkyl)NH-gamma glutamic acid-[COCH₂(OCH₂CH₂)ₖ—NH]_{q}-gamma glutamic acid-CO(CH₂)₁₄₋₂₀COOH;
R₂, R₄ and R₈ are each H;
R₃ is CH₃; and
R₅ is NH₂;
k is an integer selected from the range of 1-4; and
q is an integer selected from the range of 1-4, optionally wherein k is 1 or 2 and q is 1, 2 or 4, optionally wherein k and q are both 2.

In accordance with one embodiment the present invention provides a PTH peptide selected from the group comprising SEQ ID NO: 77, 78, 79, 84, 87, 95, 102, 108, 110, 127, or 128. In some embodiments the PTH peptide is selected from the group consisting of SEQ ID NO: 87, 95, 108, 127, or 128. In some embodiments the PTH peptide of the present invention is selected from 77 or 87. In one embodiment the PTH peptide is SEQ ID NO: 77. In another embodiment the PTH peptide is SEQ ID NO: 87. In one embodiment the present invention encompasses a PTH peptide listed in Table 2. In another embodiment the present invention encompasses a PTH peptide from Table 2 with an ornithine substitution made for one lysine in the sequence. In another embodiment the provided herein are PTH peptides from Table 2 with more than one ornithine substituted for more than one lysine.

In accordance with some embodiments the present invention provides a PTH peptide prodrug that, when administered to a patient in need thereof, provides one half or less active drug to prodrug after one week. In accordance with some embodiments the present invention provides a PTH peptide prodrug that, when administered to a patient in need thereof, provides one third or less active drug to prodrug after one week. In accordance with some embodiments the present invention provides a PTH peptide prodrug that, when administered to a patient in need thereof, provides one quarter or less active drug to prodrug after one week.

The present disclosure also encompasses other conjugates in which PTH conjugates of the present disclosure are linked, optionally via covalent bonding and optionally via a linker, to an additional conjugate moiety. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other.

The disclosed PTH peptide conjugates are believed to be suitable for any use that has previously been described for its corresponding parent PTH. Accordingly, the PTH conjugates can be administered to patients to treat any disease or conditions associated with insufficient levels of PTH (hypoparathyroidism), or disease responsive to PTH therapy such as osteoporosis. In accordance with one embodiment a method of treating hypoparathyroidism is provided wherein a patient in need of such therapy is administered a composition or conjugate in accordance with any of those described herein in an amount effective to treat or prevent hypoparathyroidism or alleviate a medical condition associated with hypoparathyroidism. In one embodiment the route of administration is parenteral, including subcutaneous. In another embodiment the route of administration is oral. In another embodiment the rout of administration is pulmonary. In certain embodiments PTH peptides of the present invention are inhaled as an aerosol, a mist or a powder formulation. Inhaled pharmaceutical compositions described herein can be delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. Such methods include those described in U.S. Pat. No. 6,468,798, herein incorporated in its entirety by reference. In certain embodiments the dosage unit of PTH peptide is determined by providing a valve to deliver a metered amount.

In certain embodiments of this disclosure, a method of treatment of osteoporosis is described. The method comprises administering any of the PTH conjugates of the present disclosure to a patient in need thereof, optionally wherein the PTH conjugate is administered in conjunction with a short acting PTH agonist (e.g. a PTH peptide of SEQ ID NO; 7, or any of SEQ ID Nos: 31-37). In one embodiment the patient is administered a composition comprising a PTH conjugate of the present disclosure and a second component selected from the group consisting of Teriparatide (Forteo®), SEQ ID NO: 7, 31, 32, 33, 34, 35, 36, 37 and calcitonin. In one embodiment the method of treating osteoporosis or osteopenia comprises administering a composition comprising a PTH conjugate of the present disclosure by daily subcutaneous injection or daily oral administration to a subject in need thereof.

In some embodiments, the subject in need of treatment has osteoporosis. In some embodiments, the subject in need of treatment has osteopenia. In certain embodiments, the subject in need of treatment is a post-menopausal woman. In some embodiments, the subject in need of treatment has glucocorticoid induced osteoporosis. In certain embodiments, the subject in need of treatment has glucocorticoid induced osteopenia. In another embodiment a method of treatment of osteoporosis is provided comprising treating a subject in need thereof by daily subcutaneous injection with a PTH conjugate of the present disclosure. In another embodiment a method of treatment of osteoporosis is provided comprising treating a subject in need thereof by every other day subcutaneous injection with a PTH conjugate of the present disclosure. In another embodiment this disclosure provides a method of treatment of osteoporosis comprising treating a subject in need thereof by weekly or monthly subcutaneous injection with a PTH conjugate of the present disclosure.

Pharmaceutical compositions comprising the conjugates disclosed herein can be formulated and administered to patients using standard pharmaceutically acceptable carriers and routes of administration known to those skilled in the art. Accordingly, the present disclosure also encompasses pharmaceutical compositions comprising one or more of the conjugates disclosed herein, or a pharmaceutically acceptable salt thereof, in combination with a pharmaceutically acceptable carrier.

In accordance with one embodiment a pharmaceutical composition is provided comprising any of the novel dipeptide/PTH peptide complexes disclosed herein, preferably sterile and preferably at a purity level of at least 90%, 91%, 92%, 93%, 94%, 95%, 96%, 97%, 98% or 99%, and a pharmaceutically acceptable diluent, carrier or excipient. Such compositions may contain a dipeptide/PTH peptide conjugate as disclosed herein, wherein the resulting active agent is present at a concentration of at least 0.1-10 mg/ml or higher. In one embodiment the pharmaceutical compositions comprise aqueous solutions that are sterilized and optionally stored within various containers. The compounds disclosed herein can be used in accordance with one embodiment to prepare pre-formulated solutions ready for injection. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

In accordance with one embodiment pharmaceutical compositions are provided wherein the disclosed prodrug forms of PTH conjugates are selected to provide an optimized level of active PTH in the patient's blood/serum/plasma. In some embodiments after a first administration of a prodrug form of one of the PTH conjugate peptides of the present disclosure to a patient in need thereof, the plasma concentration of the prodrug remains higher than the concentration of the released drug for at least 48-96 hours. In certain embodiments after initial dosing with a prodrug form of one of the PTH conjugate peptides of the present disclosure the plasma concentration of prodrug is higher than the plasma concentration of the active drug for at least 120 hours. In certain embodiments peptides of the present disclosure are administered to a patient in need thereof and the plasma concentration of active peptide peaks 48-120 hours after the initial dose. In some embodiments a prodrug form of one of the PTH conjugate peptides of the present disclosure is administered to a patient in need thereof and the plasma concentration of active peptide remains greater than 75% of its Cmax one week after administration. In some embodiments a prodrug form of one of the PTH conjugate peptides of the present disclosure is administered to a patient in need thereof and the plasma concentration of the resulting active peptide remains greater than 50% of its Cmax ten days after administration. In some embodiments a prodrug form of one of the PTH conjugate peptides of the present disclosure is administered to a patient in need thereof and the plasma concentration of resulting active peptide remains greater than 25% of its Cmax two weeks after administration.

The PTH conjugates of the present disclosure can be administered to patients using any of the known standard routes, alone or in combination with other suitable components.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The analog of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropyl methylcellulose, or carboxymethylcellulose, or emulsifying agents and other pharmaceutical adjuvants.

Administration of a compound of the present disclosure may be done in combination with shorter-acting PTH or PTH analogs. Peptides of the present disclosure may be administered to a patient in need thereof in sequence or coadministration with PTH compounds of a relatively short biological half-life.

In other embodiments, a compound of the disclosure may be administered alone or in combination with other agents, for example, bone antiresorptive agents, including calcitonin, bisphosphonates, SERMs (e.g. raloxifene), hormone replacement therapy (HRT), calcium, Vitamin D1, Vitamin D2, Vitamin D3, Vitamin D4 and estrogen. A compound of this disclosure may be co-administered with another agent. Alternatively, a compound of this disclosure may be administered sequentially with another agent; for example a compound of this disclosure is administered alone for a period from one week to one year followed by administration of another agent, either together with said compound or in the absence of said compound.

In accordance with one embodiment a pharmaceutical composition is provided comprising a PTH conjugate of the present disclosure and one or more bone antiresorptive agents, including calcitonin, bisphosphonates, SERMs (e.g. raloxifene), hormone replacement therapy (HRT), calcium, Vitamin D1, Vitamin D2, Vitamin D3, Vitamin D4 and estrogen. In one embodiment such a composition is administered to patients to treat osteopenia or osteoporosis, optionally wherein the pharmaceutical composition is formulated for oral administration.

Formulations suitable for oral administration can consist of (a) liquid solutions, such as an effective amount of the analog of the present disclosure dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and suitable emulsions. In accordance with one embodiment formulations suitable for oral administration comprises a PTH conjugate of the present disclosure and an absorption enhancer, such as sodium N-[8-(2-hydroxybenzoyl)amino]caprylate (SNAC). Sodium N-[8-(2-hydroxybenzoyl)amino]caprylate (SNAC) is a delivery agent that has been reported to enhance the permeability of a diverse spectrum of molecules, including proteins, such as insulin, calcitonin and other macromolecules such as heparin. In accordance with one embodiment pharmaceutical compositions are provided for oral delivery wherein the composition comprises a PTH conjugate of the present disclosure and SNAC, optimally wherein the pharmaceutical composition is formulated as a tablet.

All therapeutic methods, pharmaceutical compositions, kits, and other similar embodiments described herein contemplate that the dipeptide/PTH peptide complexes include all pharmaceutically acceptable salts thereof.

In one embodiment the kit is provided with a device for administering the dipeptide/PTH peptide complex composition to a patient. The kit may further include a variety of containers, e.g., vials, tubes, bottles, and the like. Preferably, the kits will also include instructions for use. In accordance with one embodiment the device of the kit is an aerosol dispensing device, wherein the composition is prepackaged within the aerosol device. In another embodiment the kit comprises a syringe and a needle, and in one embodiment the prodrug composition is prepackaged within the syringe or injection pen, using a low gauge needle, for example having a 29-31 size.

In accordance with one embodiment a modified PTH peptide is provided comprising a sequence of SEQ ID NO: 31, 32 or 33 or a sequence that differs from SEQ ID NO: 31, 32 or 33 by a lysine substitution at one or two position selected from 13, 16, 19, 22, 26 and 33, wherein said modified PTH peptide comprises an acylated amino acid at one or two position selected from 13, 16, 19, 22, 26 and 33, optionally wherein the acylated amino acid has a C16-C20 fatty acid or C16-C20 diacid covalently linked to its amino acid side chain, optionally via a spacer, and the PTH peptide further comprises a dipeptide A-B covalently linked via an amide bond to the N-terminus of the PTH peptide wherein A is an amino acid (e.g., Lys, ornithine, cysteine or homocysteine), optionally an amino acid in the D-stereochemical configuration, and optionally is an acylated amino acid; and B is an N-alkylated amino acid, optionally N-methyl glycine or N-methyl alanine.

In accordance with embodiment 1 a conjugate comprising a PTH peptide and a self-cleaving dipeptide covalently bound to said PTH peptide via an amide bond is provided, optionally wherein the self-cleaving dipeptide is linked via an amide bond to the N-terminal alpha amine of the PTH peptide, wherein
said PTH peptide comprises an amino acid sequence selected from the group consisting of

(SEQ ID NO: 133);
SVSEIQLMHX₁₀LGX₁₃HLX₁₆SX₁₈ERVEWLRX₂₆X₂₇LQDX₃₁H-Z,
(SEQ ID NO: 134);
SVSEIQLMHX₁₀LX₁₂KHLX₅₆X₁₇X₁₈ERVEWLRKKLQDVH-Z;
(SEQ ID NO: 135(
SVSEIQLMHX₁₀LGKHLX₁₆SX₁₈ERVEWLRKKLQDVH-Z
and
(SEQ ID NO: 7)
SVSEIQLMHNLGKHLNSMERVEWLRKKLQDVH-Z;

wherein
   Z is X₃₃, X₃₃F, X₃₃FX₃₅, X₃₃FVX₃₅ (SEQ ID NO: 136), X₃₃FVAX₃₅ (SEQ ID NO: 137), X₃₃FVALX₃₅ (SEQ ID NO: 138), X₃₃FVALGX₃₅ (SEQ ID NO: 139), X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally wherein Z is X₃₃, X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143);
X₁₀ and X₁₆ are independently Asp, Gln or Asn;
X₁₂ is Gly or Aib;
X₅₆ is amino isobutyric acid (Aib) or Asn;
X₁₇ is amino isobutyric acid (Aib) or Ser;
X₁₈ is Met, Met(O), Leu, or Nleu;
X₁₃, X₂₆, and X₂₇ are independently selected from the group consisting of Arg, Glu, Asp and Lys;
X₃₁ is Gly or Val;
X₃₃ and X₃₅ each comprise an acylated amino acid comprising a C16-C30 fatty acid or C16-C30 diacid covalently linked to the side chain of the amino acid, optionally via a spacer, optionally wherein the acylated amino acid is selected from the group consisting of Lys, dLys, ornithine, Cys and homocysteine;
X₅₃ is Gln or Asn, optionally with the proviso that no more than one of X₁₂, X₁₆ and X₁₇ is Aib, and optionally wherein the C-terminal amino acid is modified to replace the carboxy terminus with an amide; and
said self-cleaving dipeptide comprises the structure A-B where
A is an amino acid, optionally an acylated amino acid comprising a C16-C30 fatty acid or C16-C30 diacid covalently linked to the side chain of the amino acid, optionally via a spacer; and
B is an N-alkylated amino acid.

In accordance with embodiment 2 a conjugate of embodiment 1 is provided wherein A is selected from the group consisting of Lys, dLys, epsilon-acylated-Lys, epsilon-acylated-dLys, ornithine, epsilon-acylated ornithine, cysteine, S-acylated cysteine, homocysteine and S-acylated homocysteine, optionally wherein A is selected from the group consisting of Lys, dLys, acylated-Lys and acylated-dLys. In another embodiment a conjugate of embodiment 1 is provided wherein A is dLys. In another embodiment a conjugate of embodiment 1 is provided wherein A is epsilon-acylated-dLys. In another embodiment a conjugate of embodiment 1 is provided wherein A is selected from l-Lys or epsilon-acylated l-Lys.

In accordance with embodiment 3 a conjugate of embodiment 1 or 2 is provided wherein Z is Lys; X₁₀ and X₁₆ are Asn; X₁₃, X₂₆, and X₂₇ are Lys; X₁₇ is Ser; X₁₈ is Met; X₃₁ is Val; X₃₃ is Lys; A is dLys wherein the side chain is acylated with COC₁₆H₃₂CO₂H via a gamma Glu-COCH₂(OCH₂CH₂)₂NH-gamma Glu spacer; and B is n-methyl glycine.

In accordance with embodiment 4 a conjugate comprising a PTH peptide and a self-cleaving dipeptide covalently bound to said PTH peptide via an amide bond, optionally at the N-terminal alpha amine of the PTH protein, is provided wherein said PTH peptide comprises an amino acid sequence selected from the group consisting of
I)

(SEQ ID NO: 5);
SVSEIQLMHNLGX₁₃HLNSMERVEWLRX₂₆X₂₇LQDX₃₁H-Z,
(SEQ ID NO: 6);
SVSEIQLMHNLX₁₂KHLX₅₆X₁₇MERVEWLRKKLQDVH-Z;
and
(SEQ ID NO: 7)
SVSEIQLMHNLGKHLNSMERVEWLRKKLQDVH-Z;

wherein
Z is X₃₃, X₃₃F, X₃₃FX₃₅, X₃₃FVX₃₅ (SEQ ID NO: 136), X₃₃FVAX₃₅ (SEQ ID NO: 137), X₃₃FVALX₃₅ (SEQ ID NO: 138), X₃₃FVALGX₃₅ (SEQ ID NO: 139), X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143), optionally wherein Z is X₃₃, X₅₃X₃₅, X₅₃FX₃₅, X₅₃FVX₃₅ (SEQ ID NO: 140), X₅₃FVAX₃₅ (SEQ ID NO: 141), X₅₃FVALX₃₅ (SEQ ID NO: 142), or X₅₃FVALGX₃₅ (SEQ ID NO: 143);
X₁₂ is amino isobutyric acid (Aib) or Gly;
X₅₆ is amino isobutyric acid (Aib) or Asn;
X₁₇ is amino isobutyric acid (Aib) or Ser;
X₁₃, X₂₆, and X₂₇ are independently selected from the group consisting of Arg, Glu, Asp and Lys, optionally wherein X₁₃, X₂₆, and X₂₇ are independently selected from Glu and Lys;
X₃₁ is Gly or Val;
X₃₃ and X₃₅ each comprise an acylated amino acid comprising a C16-C30 fatty acid or C16-C30 diacid covalently linked to the side chain of the amino acid, optionally via a spacer;
X₅₃ is Gln or Asn, optionally with the proviso that only one of X₁₂, X₁₆ and X₁₇ is Aib, and optionally wherein the C-terminal amino acid is modified to replace the carboxy terminus with an amide; or
II)

(SEQ ID NO: 2)
SVSEIQLMHNLGKHLNSMERVEWLRKKLQDVHX₃₅;
(SEQ ID NO: 12)
SVSEIQLMHNLGKHLNSMERVEWLRKKLQDVHNFX₃₅;
(SEQ ID NO: 14)
SVSEIQLMHNLGKHLNSMERVEWLRKKLQDVHQFX₃₅;
(SEQ ID NO: 103)
SVSEIQLMHNLX₁₂X₁₃HLX₁₆X₁₇MERVEWLRX₂₆X₂₇LQDX₃₁HN;
(SEQ ID NO: 104)
SVSEIQLMHNLGX₁₃HLNSMERVEWLRX₂₆X₂₇LQDX₃₁HN;
(SEQ ID NO: 105)
SVSEIQLMHNLX₁₂KHLX₁₆X₁₇MERVEWLRKKLQDVHN;
(SEQ ID NO: 106)
SVSEIQLMHNLGKHLNSMERVEWLRKKLQDVHN;

wherein
X₁₂ is amino isobutyric acid (Aib) or Gly;
X₁₆ is amino isobutyric acid (Aib) or Asn;
X₁₇ is amino isobutyric acid (Aib) or Ser;
X₁₃, X₂₆, and X₂₇ are independently selected from the group consisting of Arg, Glu, Asp and Lys;
X₃₁ is Gly or Val; and
X₃₅ is an acylated amino acid comprising a C16-C30 fatty acid or C16-C30 diacid covalently linked to the side chain of the amino acid, optionally via a spacer
wherein the PTH peptide of SEQ ID NO: 2, 12, 14, 103, 104, 105, 106 or 107 is modified a substitution at any one of positions 13, 16, 19, 22, 26, 33 or at the C-terminal amino acid of the peptide of SEQ ID NO: 103, 104, 105, 106 or 107 with a lysine acylated with a C14-C20 fatty acid or C14-C20 diacid, optionally via a spacer; and
said self-cleaving dipeptide comprises the structure A-B where
   A is an amino acid, optionally an amino acid in the D-stereochemical configuration, and optionally wherein the side chain of the “A” amino acid is acylated with a C16-C30 fatty acid or C16-C30 diacid optionally via a spacer; and
   B is an N-alkylated amino acid, optionally an N—(C₁-C₄) alkylated glycine, N-methyl glycine or N-methyl alanine; optionally with the proviso that the acylated amino acid at X₃₃, X₃₅ and the “A” amino acid of the dipeptide of Formula I are the same or different, optionally wherein the amino acid at X₃₃, X₃₅ and the “A” amino acid of the dipeptide of Formula I are both lysine but differ in the spacer, stereochemistry or the acylating group attached to the lysine side chain, optionally wherein “A” is an epsilon-acylated dLys and X₃₃, and X₃₅ are each an epsilon-acylated Lys, optionally wherein the acylating group is a C16-C30 fatty acid or C16-C30 diacid optionally linked via a spacer.

In accordance with embodiment 5, a conjugate of any one of embodiments 1-2 or 4 is provided wherein each of the acylated amino acids of the conjugate comprises a C16-C30 fatty acid or C16-C30 diacid linked via a spacer wherein the spacer comprises one or more linker moieties independently selected from the group consisting of a gamma glutamic acid, and COCH₂(OCH₂CH₂)ₖNH, wherein k is an integer selected from the range of 1-8 with the optional proviso that when A is a non-acylated amino acid, then A is an amino acid in the D-stereochemical configuration.

In accordance with embodiment 6 a conjugate of embodiment 1 is provide wherein A is selected from the group consisting of Lys, dLys, acylated-Lys, acylated-dLys, ornithine, acylated omithine, cysteine, acylated cysteine, homocysteine and acylated homocysteine, optionally wherein A is selected from the group consisting of Lys, dLys, acylated-Lys and acylated-dLys.

In accordance with embodiment 7 a conjugate of any one of embodiments 1-6 is provided wherein Z is X₃₃, X₅₃X₃₅, or X₅₃FX₃₅;

In accordance with embodiment 8 a conjugate comprising a PTH peptide and a self-cleaving dipeptide covalently bound to said PTH peptide via an amide bond, optionally at the N-terminal alpha amine of the PTH protein, is provided wherein,
said PTH peptide comprising an amino acid sequence selected from the group consisting of
SVSEIQLMHNLGX₁₃HLNSMERVEWLRX₂₆X₂₇LQDX₃₁H-Z, (SEQ ID NO: 5);
SVSEIQLMHNLX₁₂KHLX₅₆X₁₇MERVEWLRKKLQDVH-Z; (SEQ ID NO: 6);
SVSEIQLMHNLGKHLNSMERVEWLRKKLQDVH-Z (SEQ ID NO: 7), and a peptide that differs from the peptide of SEQ ID NO: 7 by 1, 2 or 3 amino acid substitutions;
   wherein
   Z is X₃₃F, X₅₃X₃₅, X₅₃FX₃₅, or X₃₃;
   X₁₂ is amino isobutyric acid (Aib) or Gly;
   X₅₆ is amino isobutyric acid (Aib) or Asn;
   X₁₇ is amino isobutyric acid (Aib) or Ser;
   X₁₃, X₂₆, and X₂₇ are independently selected from the group consisting of Arg, Glu, Asp and Lys;
   X₃₁ is Gly or Val;
   X₃₃ and X₃₅ each comprise an acylated amino acid;
   X₅₃ is Gln or Asn; and
   said self-cleaving dipeptide comprising the general structure

A-B—;
wherein
A is an amino acid or an acylated amino acid, optionally wherein the amino acid or acylated amino acid is in the D-stereochemical configuration;
B is an N-alkylated amino acid;
wherein said acylated amino acid of each of X₃₃, X₃₅ and A is independently selected from an amino acid comprising a C16-C30 fatty acid or C16-C30 diacid covalently linked to its amino acid side chain, optionally via a spacer, and said self-cleaving dipeptide is linked to said PTH peptide through formation of an amide bond between B and the N-terminal alpha amine of said PTH peptide or at any one of positions 13, 16, 19, 22, 26 and 33, wherein said optional spacer of the acylated amino acids of X₃₃, X₃₅ and A is independently selected from the group consisting of a gamma glutamic acid, a gamma glutamic acid-gamma glutamic acid dipeptide, and a gamma glutamic acid-[COCH₂(OCH₂CH₂)ₖ—NH]_{q}-gamma glutamic acid, wherein k is an integer selected from the range of 1-8 and q is an integer selected from the range of 1-4; and
with the proviso that when A is a non-acylated amino acid, then A is an amino acid in the D-stereochemical configuration.

In accordance with embodiment 9 a conjugate of embodiment 8 is provided wherein said PTH peptide comprises the sequence

(SEQ ID NO: 7)
SVSEIQLMHNLGKHLNSMERVEWLRKKLQDVH-Z,

wherein
Z is X₃₃F, X₅₃X₃₅, X₅₃FX₃₅, or X₃₃;
X₃₃ and X₃₅ are each independently an amino acid comprising a C16-C30 fatty acid or C16-C30 diacid covalently linked to the acid side chain of the amino acid, optionally via a spacer; and
X₅₃ is Asn with the optional proviso that the acylated amino acid at X₃₃, X₃₅ and the “A” amino acid of the dipeptide of Formula I are the same or different, optionally wherein the amino acid at X₃₃, X₃₅ and the “A” amino acid of the dipeptide of Formula I are each lysine but differ in the spacer or the acylating group attached to the lysine side chain, optionally wherein the acylated amino acid at X₃₃, X₃₅ and the “A” amino acid of the dipeptide of Formula I are identical.

In accordance with embodiment 10 a conjugate of any one of embodiments 4-9 is provided wherein said PTH peptide comprises the sequence of

(SEQ ID NO: 16)
SVSEIQLMHNLGKHLNSMERVEWLRKKLQDVHX₃₃
or
(SEQ ID NO: 12)
SVSEIQLMHNLGKHLNSMERVEWLRKKLQDVHNFX₃₅

wherein X₃₃ and X₃₅ are each independently an amino acid comprising a C16-C30 fatty acid or C16-C30 diacid covalently linked to the acid side chain of the amino acid, optionally via a spacer.

In accordance with embodiment 11 conjugate of any one of embodiments 1-10 is provided wherein the acylated amino acid of A, X₃₃ and X₃₅ is independently selected from cysteine, homocysteine, ornithine and lysine wherein the side chain of said cysteine, homocysteine, omithine or lysine is covalently linked to a C16-C22 fatty acid or C16-C22 diacid, optionally through a spacer comprising a gamma glutamic acid linkage.

In accordance with embodiment 12 a conjugate of any one of embodiments 1-11 is provided wherein the acylated amino acid of A, X₃₃ and X₃₅ are each lysine or d-lysine wherein the side chain of said lysine or d-lysine is covalently linked to a C16-C22 fatty acid or C16-C22 diacid, optionally through a spacer comprising a gamma glutamic acid linkage.

In accordance with embodiment 13 a conjugate of any one of embodiments 1-12 is provided wherein A is selected from the group consisting of Lys, dLys, epsilon-acylated-Lys and epsilon-acylated-dLys wherein said self-cleaving dipeptide is covalently linked to the N-terminal alpha amine of the PTH peptide via the carboxy terminus of the B amino acid.

In accordance with embodiment 14 a conjugate of any one of embodiments 1-13 is provided wherein said acylated amino acid of each of X₃₃, X₃₅ and A comprises a C16-C30 fatty acid or C16-C30 diacid covalently linked to the amino acid side chain via a spacer, wherein the spacer of each of X₃₃, X₃₅ and A is independently selected from a gamma glutamic acid-gamma glutamic acid dipeptide, (Xaa)-[COCH₂(OCH₂CH₂)ₖNH]_{q}-gamma glutamic acid, a gamma glutamic acid-[COCH₂(OCH₂CH₂)ₖNH]_{q}-gamma glutamic acid, wherein
Xaa is selected from Arg, Tyr(OPO₃H₂), and hCys(SO₃H);
k is an integer selected from the range of 1-8; and
q is an integer selected from the range of 1-8, optionally wherein k is 2 and q is selected from the range of 1-4}, optionally wherein the spacer is -{gamma glutamic acid-[COCH₂(OCH₂CH₂)₂NH—COCH₂(OCH₂CH₂)₂]NH-gamma glutamic acid}-.

In accordance with embodiment 15 a conjugate of any one of embodiments 1-14 is provided wherein said spacer comprises the structure: gamma glutamic acid-[COCH₂(OCH₂CH₂)ₖ—NH]_{q}-gamma glutamic acid, wherein
k is an integer selected from the range of 1-8; and
q is an integer selected from the range of 1-8, optionally wherein k is 2, 4, 6 or 8 and q is 1, optionally wherein k is 2 or 4 and q is 2 or 4, optionally wherein k is 2 or 4 and q is 1, optionally wherein k is 2 and q is selected from the range of 1-8, optionally wherein k is 2 and q is 2.

In accordance with embodiment 16 a conjugate of any one of embodiments 1-15 is provided wherein A-B comprises the structure:

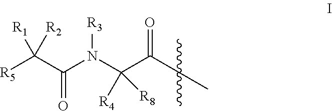

wherein
R₁, comprises a side chain selected from the group consisting of C₁-C₈ alkyl, (C₁-C₄ alkyl)OH, (C₁-C₄ alkyl)SH, (C₁-C₄ alkyl)COOH, and (C₁-C₄ alkyl)NH₂, optionally wherein a C16-C30 fatty acid or C16-C30 diacid is covalently linked to said side chain, optionally via said spacer;
R₂, R₄ and R₈ are independently H, or C₁-C₄ alkyl;
R₃ is C₁-C₆ alkyl; and
R₅ is NH₂, wherein said spacer comprises an amino acid or dipeptide.

In accordance with embodiment 17 a conjugate of any one of embodiments 1-16 is provided wherein said spacer is selected from the group consisting of a gamma glutamic acid, a gamma glutamic acid-gamma glutamic acid dipeptide, and a gamma glutamic acid-[COCH₂(OCH₂CH₂)ₖ—NH]_{q}-gamma glutamic acid, wherein k is an integer selected from the range of 2-4 and q is 1 or 2, optionally wherein the spacer is -{gamma glutamic acid-[COCH₂(OCH₂CH₂)₂NH—COCH₂(OCH₂CH₂)₂]NH-gamma glutamic acid}-.

In accordance with embodiment 18 a conjugate of any one of embodiments 1-17 is provided wherein the chemical cleavage half-life (t_{1/2}) of A-B from said PTH peptide is at least about 48 to 168 hours in standard PBS solution under physiological conditions.

In accordance with embodiment 19 a conjugate of any one of embodiments 1-17 is provided wherein the chemical cleavage half-life (t_{1/2}) of A-B from said PTH peptide is at least about 70 to 140 hours in standard PBS solution under physiological conditions.

In accordance with embodiment 20 a conjugate of any one of embodiments 1-17 is provided wherein the chemical cleavage half-life (t_{1/2}) of A-B from said PTH peptide is at least about 90 to 120 hours in standard PBS solution under physiological conditions.

In accordance with embodiment 21, a conjugate of embodiment 16 is provided wherein
R₁, is (C₁-C₄ alkyl)NH;
R₂ and R₈ are each H;
R₄ is H, or CH₃;
R₃ is CH₃ and
R₅ is NH₂.

In accordance with embodiment 22 a conjugate of embodiment 16 is provided wherein
R₁, is (C₁-C₄ alkyl)NH-[spacer]-CO(CH₂)₁₄₋₂₀COOH, (C₁-C₄ alkyl)NH-[spacer]-CO(CH₂)₁₄₋₂₀CH₃, (C₁-C₄ alkyl)NH—CO(CH₂)₁₄₋₂₀COOH, or (C₁-C₄ alkyl)NH—CO(CH₂)₁₄₋₂₀CH₃;
R₂ and R₈ are each H;
R₄ is H, or CH₃;
R₃ is CH₃ and
R₅ is NH₂, wherein said [spacer] is a linking moiety selected from the group consisting of a gamma glutamic acid, a gamma glutamic acid-gamma glutamic acid dipeptide, and a gamma glutamic acid-[COCH₂(OCH₂CH₂)ₖNH]_{q}-gamma glutamic acid, wherein k is an integer selected from the range of 2-4 and q is an integer selected from the range of 1-8.

In accordance with embodiment 23 a conjugate of embodiment 22 is provided wherein R₁, is (C₁-C₄ alkyl)NH-[spacer]-CO(CH₂)₁₄₋₂₀COOH wherein k is 2 or 4 and q is 1, 2 or 4, optionally wherein k and q are both 2.

In accordance with embodiment 24 a conjugate of embodiment 22 is provided wherein
R₁, is (C₄ alkyl)NH-[gamma glutamic acid-[COCH₂(OCH₂CH₂)ₖ—NH]_{q}-gamma glutamic acid]-CO(CH₂)₁₄₋₂₀COOH, wherein k is 2-4 and q is 1 or 2.

In accordance with embodiment 25 a conjugate of any one of embodiments 1-24 is provided wherein the first amino acid of the cleavable dipeptide is an amino acid in the D-stereochemical configuration.

In accordance with embodiment 26 a conjugate comprising a PTH peptide and a self-cleaving dipeptide covalently linked to the N-terminal alpha amine of said PTH peptide via an amide bond is provided, wherein
said PTH peptide comprises the amino acid sequence of
SVSEIQLMHNLGKHLNSMERVEWLRKKLQDVHX₃₃ (SEQ ID NO: 16), SVSEIQLMHNLGKHLNSMERVEWLRKKLQDVHNFX₃₅ (SEQ ID NO: 12), or a peptide that differs from the peptide of SEQ ID NO: 16 or SEQ ID NO: 12 by 1 or 2 amino acid substitutions wherein
X₃₃ and X₃₅ are each an amino acid comprising a side chain of (C₁-C₄ alkyl)NH—CO(CH₂)₁₄₋₂₀COOH, (C₁-C₄ alkyl)NH-[spacer]-CO(CH₂)₁₄₋₂₀COOH, (C₁-C₄ alkyl)NH—CO(CH₂)₁₄₋₂₀CH₃ or (C₁-C₄ alkyl)NH-[spacer]-CO(CH₂)₁₄₋₂₀CH₃; and
said self-cleaving dipeptide comprises the general structure:

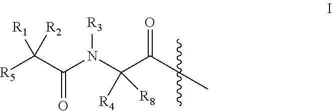

wherein
R₁, is (C₁-C₄ alkyl)NH, (C₁-C₄ alkyl)NH—CO(CH₂)₁₄₋₂₀COOH, (C₁-C₄ alkyl)NH-[spacer]-CO(CH₂)₁₄₋₂₀COOH, (C₁-C₄ alkyl)NH—CO(CH₂)₁₄₋₂₀CH₃ or (C₁-C₄ alkyl)NH-[spacer]-CO(CH₂)₁₄₋₂₀CH₃;
R₂ and R₈ are each H;
R₄ is H, or CH₃;
R₃ is C₁-C₃ alkyl and
R₅ is NH₂, wherein said spacer is selected from the group consisting of a gamma glutamic acid, a gamma glutamic acid-gamma glutamic acid dipeptide, and a gamma glutamic a-d--[COCH₂(OCH₂CH₂)ₖ—NH]_{q}-gamma glutamic acid, wherein k is an integer selected from the range of 2-4 and q is 1 or 2 optionally wherein the C-terminal amino acid is modified to replace the carboxy terminus with an amide.

In accordance with embodiment 27 a conjugate of claim 26 wherein said PTH peptide comprises the amino acid sequence of
SVSEIQLMHNLGKHLNSMERVEWLRKKLQDVHNFX₃₅ (SEQ ID NO: 12), wherein
X₃₅ is an amino acid comprising a side chain of (C₁-C₄ alkyl)NH-[gamma glutamic acid-[COCH₂(OCH₂CH₂)ₖ—NH]_{q}-gamma glutamic acid]-CO(CH₂)₁₄₋₂₀COOH. optionally wherein X₃₅ is an amino acid comprising a side chain of (C₄ alkyl)NH-{gamma glutamic acid-[COCH₂(OCH₂CH₂)₂NH—COCH₂(OCH₂CH₂)₂]NH-gamma glutamic acid}-CO(CH₂)₁₄₋₂₀COOH; and
said self-cleaving dipeptide comprises the general structure:

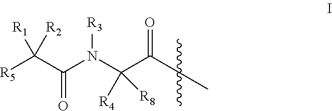

wherein
R₁, is (C₁-C₄ alkyl)NH-[gamma glutamic acid-[COCH₂(OCH₂CH₂)ₖ—NH]_{q}-gamma glutamic acid]-CO(CH₂)₁₄₋₂₀COOH;
R₂, R₄ and R₈ are each H;
R₃ is CH₃;
R₅ is NH₂;
q is 2 or 4 and
k is 2, optionally wherein q is 2, optionally wherein R₁ is (C₄ alkyl)NH-{gamma glutamic acid-[COCH₂(OCH₂CH₂)₂NH—COCH₂(OCH₂CH₂)₂]NH-gamma glutamic acid}-CO(CH₂)₁₄₋₂₀COOH with the optional proviso that the acylated amino acid at X₃₅ and the “A” amino acid of the dipeptide of Formula I are the, optionally wherein the amino acid at X₃₅ and the “A” amino acid of the dipeptide of Formula I are both lysine but differ in the spacer, stereochemistry or the acylating group attached to the lysine side chain, optionally wherein A is an epsilon-acylated dLys and X₃₅ is an epsilon-acylated Lys optionally wherein the C-terminal amino acid is modified to replace the carboxy terminus with an amide.

In accordance with embodiment 28 a conjugate of embodiment 26 is provided wherein said PTH peptide comprises the amino acid sequence of

(SEQ ID NO: 16)
SVSEIQLMHNLGKHLNSMERVEWLRKKLQDVHX₃₃,

wherein
X₃₃ is an amino acid comprising a side chain of (C₁-C₄ alkyl)NH-[gamma glutamic acid-[COCH₂(OCH₂CH₂)ₖ—NH]_{q}-gamma glutamic acid]-CO(CH₂)₁₄₋₂₀COOH, optionally an amino acid comprising a side chain of (C₄ alkyl)NH-{gamma glutamic acid-[COCH₂(OCH₂CH₂)₂NH—COCH₂(OCH₂CH₂)₂]NH-gamma glutamic acid}-CO(CH₂)₁₆₋₁₈COOH; and
said self-cleaving dipeptide comprises the general structure:

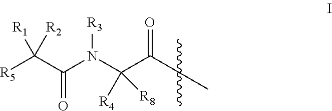

wherein
R₁, is (C₁-C₄ alkyl)NH-[gamma glutamic acid-[COCH₂(OCH₂CH₂)ₖ—NH]_{q}-gamma glutamic acid]-CO(CH₂)₁₄₋₂₀COOH, optionally (C₄ alkyl)NH-{gamma glutamic acid-[COCH₂(OCH₂CH₂)₂NH—COCH₂(OCH₂CH₂)₂]NH-gamma glutamic acid}-CO(CH₂)₁₆₋₁₈—COOH;
R₂, R₄ and R₈ are each H;
R₃ is CH₃;
R₅ is NH₂;
q is 2 and
k is 2 or 4.

In accordance with embodiment 29 a conjugate of any one of embodiments 1-28 is provided wherein the first amino acid of the self-cleaving dipeptide is in the D-stereochemical configuration and optionally said spacer when present is -{gamma glutamic acid-[COCH₂(OCH₂CH₂)₂NH—COCH₂(OCH₂CH₂)₂]NH-gamma glutamic acid}-.

In accordance with embodiment 30 a pharmaceutical composition comprising the conjugate of any one of embodiments 1-29, or pharmaceutically acceptable salt thereof, and a pharmaceutically acceptable carrier is provided.

In accordance with embodiment 31 a pharmaceutical composition of embodiment 30 is provided formulated for oral delivery wherein said composition further comprises sodium N[8-(2-hydroxybenzoyl)amino caprylate], optionally wherein the composition is formulated in a tablet form.

In accordance with embodiment 32 a pharmaceutical composition of embodiment 30 or 31 is provided further comprising a peptide of SEQ ID NO: 7, SEQ ID NO: 31, or SEQ ID NO: 32 and optionally calcitonin.

In accordance with embodiment 33 a method of treating hypoparathyroidism, is provided, wherein said method comprises administering an effective amount of a pharmaceutical composition of any one of embodiments 30, 31 or 32 to a patient in need of treatment.

In accordance with embodiment 34 a liquid pharmaceutical composition of embodiment 30 is provided formulated for intravenous, subcutaneous, or intramuscular delivery.

In accordance with embodiment 35 a method of treating osteoporosis or osteopenia is provided, wherein said method comprises administering an effective amount of a pharmaceutical composition of any one of embodiments 30, 31 or 32 to a patient in need of treatment.

In accordance with embodiment 36 a method in accordance with any one of embodiments 33-35 is provided wherein the composition is administered daily, every other day, once per week or once every two weeks.

In accordance with embodiment 37 method of any one of embodiments 33, 35-36 is provided wherein the composition is administered orally.

In accordance with embodiment 38 method of any one of embodiments 33, 35-36 is provided wherein the composition is administered via pulmonary delivery.

In accordance with embodiment 37 the use of any one of the conjugates of embodiments 1-29 for treating hypoparathyroidism or alleviating symptoms associated with hypoparathyroidism is provided.

### Example 1: Determination of Rate of Model Dipeptide Cleavage (in PBS)

A specific hexapeptide (HSRGTF-NH₂; SEQ ID NO: 28) was used as a model peptide to determine the half-life of various dipeptides linked to the hexapeptide through an amide bond. The hexapeptide was assembled on a peptide synthesizer and Boc-protected sarcosine and lysine were successively added to the model peptide-bound resin to produce peptide A (Lys-Sar-HSRGTF-NH₂; SEQ ID NO: 29). Peptide A was cleaved by HF and purified by preparative HPLC.

### Preparative Purification Using HPLC:

Purification was performed using HPLC analysis on a silica based 1×25 cm Vydac C18 (5μ particle size, 300 A° pore size) column. The instruments used were: Waters Associates model 600 pump, Injector model 717, and UV detector model 486. A wavelength of 230 nm was used for all samples. Solvent A contained 10% CH₃CN/0.1% TFA in distilled water, and solvent B contained 0.1% TFA in CH₃CN. A linear gradient was employed (0 to 100% B in 2 hours). The flow rate was 10 ml/min and the fraction size was 4 ml. From ~150 mgs of crude peptide, 30 mgs of the pure peptide were obtained.

Peptide A was dissolved at a concentration of 1 mg/ml in PBS buffer. The solution was incubated at 37° C. Samples were collected for analysis at 5 h, 8 h, 24 h, 31 h, and 47 h. The dipeptide cleavage was quenched by lowering the pH with an equal volume of 0.1% TFA. The rate of cleavage was qualitatively monitored by LC-MS and quantitatively studied by HPLC. The retention time and relative peak area for the prodrug and the parent model peptide were quantified using Peak Simple Chromatography software.

### Analysis Using Mass Spectrometry

The mass spectra were obtained using a Sciex API-III electrospray quadrupole mass spectrometer with a standard ESI ion source. Ionization conditions that were used are as follows: ESI in the positive-ion mode; ion spray voltage, 3.9 kV; orifice potential, 60 V. The nebulizing and curtain gas used was nitrogen flow rate of 0.9 L/min. Mass spectra were recorded from 600-1800 Thompsons at 0.5 Th per step and 2 msec dwell time. The sample (about 1 mg/mL) was dissolved in 50% aqueous acetonitrile with 1% acetic acid and introduced by an external syringe pump at the rate of 5 μL/min. Peptides solubilized in PBS were desalted using a ZipTip solid phase extraction tip containing 0.6 μL C4 resin, according to instructions provided by the manufacturer (Millipore Corporation, Billerica, MA) prior to analysis.

### Analysis Using HPLC

The HPLC analyses were performed using a Beckman System Gold Chromatography system equipped with a UV detector at 214 nm and a 150 mm×4.6 mm C8 Vydac column. The flow rate was 1 ml/min. Solvent A contained 0.1% TFA in distilled water, and solvent B contained 0.1% TFA in 90% CH₃CN. A linear gradient was employed (0% to 30% B in 10 minutes). The data were collected and analyzed using Peak Simple Chromatography software.

The initial rates of cleavage were used to measure the rate constant for the dissociation of the dipeptides from the respective prodrugs. The concentrations of the prodrugs and the model parent peptide were determined by their respective peak areas, ‘a’ and ‘b’ for each of the different collection times. The zero order dissociation rate constants of the prodrugs were determined by plotting the logarithm of the concentration of the prodrug at various time intervals. The slope of this plot provides the rate constant ‘k’. The half-lives for cleavage of the various prodrugs were calculated by using the formula t_{1/2}=0.693/k. The results generated in these experiments are presented in Table 1.

TABLE 1
Cleavage of the Dipeptide A-B linked to histidine (or a histidine
derivative) at position 1 (X) from the Model Hexapeptide
(XSRGTF-NH₂; SEQ ID NO: 28) in PBS
NH₂-A-B-XSRGTF-NH₂ (SEQ ID NO: 28)

| | A (amino | B (amino | X₁ (amino | | |
|---|---|---|---|---|---|
| # | acid) | acid) | acid) | t_{1/2} | |
| 1 | F | P | H | No cleavage | |
| 2 | Hydroxyl-F | P | H | No cleavage | |
| 3 | G | P | H | No cleavage | |
| 4 | Hydroxyl-G | P | H | No cleavage | |
| 5 | A | P | H | No cleavage | |
| 6 | C | P | H | No cleavage | |
| 7 | S | P | H | No cleavage | |
| 8 | P | P | H | No cleavage | |
| 9 | K | P | H | No cleavage | |
| 10 | E | P | H | No cleavage | |
| 11 | Dehydro V | P | H | No cleavage | |
| 12 | P | d-P | H | No cleavage | |
| 13 | d-P | P | H | No cleavage | |
| 14 | Aib | P | H | 32 | h |
| 15 | Aib | d-P | H | 20 | h |
| 16 | Aib | P | d-H | 16 | h |
| 17 | Cyclohexyl- | P | H | 5 | h |
| 18 | Cyclopropyl- | P | H | 10 | h |
| 19 | N-Me-Aib | P | H | >500 | h |
| 20 | α,α-diethyl-Gly | P | H | 46 | h |
| 21 | Hydroxyl-Aib | P | H | 61 | |
| 22 | Aib | P | A | 58 | |
| 23 | Aib | P | N-Methyl-His | 30 | h |
| 24 | Aib | N-Methyl-Gly | H | 49 | min |
| 25 | Aib | N-Hexyl-Gly | H | 10 | min |
| 26 | Aib | Azetidine-2- | H | >500 | h |
| | | carboxylic acid | | | |
| 27 | G | N-Methyl-Gly | H | 104 | h |
| 28 | Hydroxyl-G | N-Methyl-Gly | H | 149 | h |
| 29 | G | N-Hexyl-Gly | H | 70 | h |
| 30 | dK | N-Methyl-Gly | H | 27 | h |
| 31 | dK | N-Methyl-Ala | H | 14 | h |
| 32 | dK | N-Methyl-Phe | H | 57 | h |
| 33 | K | N-Methyl-Gly | H | 14 | h |
| 34 | F | N-Methyl-Gly | H | 29 | h |
| 35 | S | N-Methyl-Gly | H | 17 | h |
| 36 | P | N-Methyl-Gly | H | 181 | h |

### Example 2: Synthesis of PTH Conjugates

The PTH peptide analogs were assembled on a 0.1 mmol Fmoc-Lys(Mtt)-Wang resin using an ABI-433A peptide synthesizer and Fmoc/Oxyma/DIC coupling protocols. Fmoc-Sar-OH and Boc-D-Lys(Boc)-OH were sequentially coupled to the N-terminus of the native first amino acid (Ser¹). The Mtt side chain of Lys³³ was deprotected and the resultant free amine was used for additional extension. This amine was coupled sequentially with Fmoc-Glu-OtBu, and two repeat additions of Fmoc-NH-PEG₂-CH₂COOH, followed by Fmoc-Glu-OtBu, and finally octadecanedioic acid mono-tert-butyl ester. The peptide was chemically removed from the synthetic resin by treatment with a TFA solution containing 2.5% TIS, 2.5% 2-mercaptoethanol, 2.5% anisole, and 2.5% H₂O at room temperature with gentle agitation for 2 hours. The resin was removed by filtration, and the peptide precipitated by addition of cold ether (50 ml). The peptide precipitate was collected by centrifugation and washed with cold ether (3×50 ml).

The impure peptide was subjected to purification by preparative reverse-phase HPLC column (Kinetex® 5 μm C8 100 Å LC Column 250×21.2 mm, 10-50% aqueous ACN (0.1% TFA), at a flow rate of 15 mL/min). The pure peptide was assessed by analytical LCMS and pooled fractions were lyophilized to provide the final product as a white fluffy solid.

In those instances where fatty acylation with two different structures were employed the sidechain of the dipeptide-prodrug at the last lysine was coupled as Boc-D-Lys(Fmoc)-OH. The lysine Fmoc sidechain was deprotected by standard base treatment. The assembly of the fatty-acid side chain occurred as described for Lys³³. To install the second but differing fatty acid at Lys³³ the protected peptide resin was further employed as described above for the mono-fatty acylated peptide analogs where the Mtt side chain of Lys³³ was deprotected next and the lysine sidechain was used for additional chemical extension.

For the peptide analogs where double fatty acylation of the same structure was employed at the N and C-terminal lysine residues the last amino acid was added as Boc-D-Lys(Fmoc)-OH. Following full assembly of the resin the Fmoc and Mtt protecting groups were removed so that the sidechains were simultaneously extended by the common coupling procedure described above.

### PEGylation

The peptide [Cys³⁵]-PTH(1-35) was assembled on a 0.1 mmol Rink Amide resin using an ABI-433A peptide synthesizer and Fmoc/Oxyma/DIC coupling protocols. TFA cleavage and ether precipitation as described previously in preparation of peptides employed in fatty acylation provided the impure Cys³⁵-peptide, which was purified by preparative HPLC. The thiol conjugation was performed in 100 mM sodium citrate buffer, pH 4.0 containing 1 mM EDTA and 10 mM TCEP. Excess 40 k maleimido, methoxy PEG was added and the reaction mixture was stirred overnight at room temperature. Unreacted PEG reagent was removed by cation exchange chromatography using SP-Sepharose resin and a linear salt gradient with 20 mM sodium acetate pH 4.0 as buffer A and same buffer containing 1 M NaCl as buffer B. The pooled fractions were desalted with C2 SPE cartridge and lyophilized to give the desired PEGylated PTH. A listing of compounds prepared in accordance with the present disclosure is provided in Table 2.

TABLE 2
PTH Analogs

| Sequence | Description | SEQ ID NO |
|---|---|---|
| SVSEIQLMHNLGKHLNSMERVEWLRKKLQDVHNF-OH | PTH(1-34) | 32 |
| SVSEIQLMHNLGKHLNSMERVEWLRKKLQDVHNFa-NH2 | PTH(1-35)a, dA35 | 40 |
| VSEIQLMHNLGKHLNSMERVEWLRKKLQDVHNF-OH | PTH(2-34) | 41 |
| GVSEIQLMHNLGKHLNSMERVEWLRKKLQDVHNF-OH | PTH(1-34), G1 | 42 |
| AVSEIQLMHNLGKHLNSMERVEWLRKKLQDVHNF-OH | PTH(1-34), A1 | 43 |
| k(γE-COC16H32CO2H)G | PTH(1-34), | 44 |
| SVSEIQLMHNLGKHLNSMERVEWLRKKLQDVHNF-OH | dK-1(γE- | |
| | diacidC18), G0 | |
| k(γE-COC16H32CO2H)(N-Me)G | PTH(1-34), | 45 |
| SVSEIQLMHNLGKHLNSMERVEWLRKKLQDVHNF-OH | dK-1(γE-diacidC18), | |
| | N(Me)G0 | |
| k(γE-COC16H32CO2H)(N-iPr)G | PTH(1-34), | 46 |
| SVSEIQLMHNLGKHLNSMERVEWLRKKLQDVHNF-OH | dK-1(γE-diacidC18), | |
| | N(iPr)G0 | |
| kGSVSEIQLMHNLGKHLNSMERVEWLRKKLQDVHNF- | PTH(1-34), | 47 |
| OH | dK-1, G0 | |
| K(γE-COC16H32CO2H)(N-iPr)G | PTH(1-34), K- | 48 |
| SVSEIQLMHNLGKHLNSMERVEWLRKKLQDVHNF-OH | 1(γE-diacidC18), | |
| | N(iPr)G0 | |
| k(γE-COC16H32CO2H)(N-iPr)G | PTH(1-34), | 49 |
| SVSEIQLMHNLGKHLNSMERVEWLRKKLQDVHNF-OH | dK-1(γE-diacidC18), | |
| | N(iPr)G0, dS2 | |
| k(γE-COC16H32CO2H)P | PTH(1-34), | 50 |
| SVSEIQLMHNLGKHLNSMERVEWLRKKLQDVHNF-OH | dK-1(γE- | |
| | diacidC18), P0 | |
| K(γE-COC16H32CO2H)P | PTH(1-34), K-1(γE- | 51 |
| SVSEIQLMHNLGKHLNSMERVEWLRKKLQDVHNF-OH | diacidC18), P0 | |
| K(γE-COC16H32CO2H)(N-Me)G | PTH(1-34), K- | 52 |
| SVSEIQLMHNLGKHLNSMERVEWLRKKLQDVHNF-OH | 1(γE- | |
| | diacidC18), N(Me)G0 | |
| K(γE-COC16H32CO2H)(N-Me)A | PTH(1-34), K- | 53 |
| SVSEIQLMHNLGKHLNSMERVEWLRKKLQDVHNF-OH | 1(γE-diacidC18), | |
| | N(Me)A0 | |
| K(γE-COC16H32CO2H)(Pip) | PTH(1-34), K- | 54 |
| SVSEIQLMHNLGKHLNSMERVEWLRKKLQDVHNF-OH | 1(γE- | |
| | diacidC18), Pip0 | |
| SVSEIQLMHNLGKHLNSMERVEWLRKKLQDVHQF-OH | PTH(1-34), Q33 | 55 |
| SVSEIQLMHNLGKHLNSMERVEWLRKKLQDVHNFK | PTH(1-35)a, | 56 |
| (γE-COC16H32CO2H)-NH2 | K35(γE-diacidC18) | |
| k(N-Me)G | PTH(1-35)a, | 57 |
| SVSEIQLMHNLGKHLNSMERVEWLRKKLQDVHNFK | dK-1, N(Me)G0, | |
| (γE-COC16H32CO2H)-NH2 | K35(γE-diacidC18) | |
| k(γE-COC16H32CO2H)(N-Me)G | PTH(1-35)a, | 58 |
| SVSEIQLMHNLGKHLNSMERVEWLRKKLQDVHNFK | dK-1(γE- | |
| (γE-COC16H32CO2H)-NH2 | diacidC18), | |
| | N(Me)G0, | |
| | K35(γE-diacidC18) | |
| SVSEIQLMHNLGKHLNSMERVEWLRKKLQDVHN-OH | PTH(1-33) | 59 |
| SVSEIQLMHNLGKHLNSMERVEWLRKKLQDVHD-NH2 | PTH(1-33)a, D33 | 60 |
| k(γE-COC16H32CO2H)(N-Me)G | PTH(1-34), | 61 |
| SVSEIQLMHNLGKHLNSMERVEWLRKKLQDVHQF-OH | dK-1 (γE-diacidC18), | |
| | N(Me)G0, Q33 | |
| QLMHNLGKHLNSMERVEWLRKKLQDGHNF-OH | PTH(6-34), G31 | 62 |
| SEIQLMHNLGKHLNSMERVEWLRKKLQDGHNF-OH | PTH(3-34), G31 | 97 |
| SVSEIQLMHNLGKHLNSMERVEWLRKKLQDGHNF-OH | PTH(1-34), G31 | 98 |
| QLMHNLGKHLNSMERVEWLREKLQDVHNF-OH | PTH(6-34), E26 | 99 |
| SEIQLMHNLGKHLNSMERVEWLREKLQDVHNF-OH | PTH(3-34), E26 | 100 |
| SVSEIQLMHNLGKHLNSMERVEWLREKLQDVHNF-OH | PTH(1-34), E26 | 101 |
| SVSEIQLMHNLGKHLNSMERVEWLRKKLQDVHK(γE- | PTH(1-34), | 102 |
| COC16H32CO2H)F-OH | K33(γE-diacidC18) | |
| SVSEIQLMHNLGKHLNSMERVEWLRKKLQDVHNFVAL | PTH(1-38)a, | 63 |
| K(γE-COC16H32CO2H)-NH2 | K38(γE-diacidC18) | |
| SVSEIQLMHNLGRHLNSMERVEWLRRRLQDVHK | PTH(1-34), | 64 |
| (γE-COC16H32CO2H)F-OH | R13, R26, R27, | |
| | K33(γE-diacidC18) | |
| k(γE-(miniPEG)2-γE-COC16H32CO2H)G | PTH(1-35)a, dK-1(γE- | 65 |
| SVSEIQLMHNLGKHLNSMERVEWLRKKLQDVHNFK | 2xOEG-γE- | |
| (γE-(miniPEG)2-γE-COC16H32CO2H)-NH2 | diacidC18), G0, | |
| | K35(γE-2xOEG-γE- | |
| | diacidC18) | |
| SVSEIQLMHNLGKHLNSMERVEWLRKELQDVHNFK | PTH(1-35)a, | 66 |
| (γE-COC16H32CO2H)-NH2 | E27, K35(γE- | |
| | diacidC18) | |
| SVSEIQLMHNLGKHLNSMERVEWLREKLQDVHNFK | PTH(1-35)a, | 67 |
| (γE-COC16H32CO2H)-NH2 | E26, K35(γE- | |
| | diacidC18) | |
| SVSEIQLMHNLGEHLNSMERVEWLRKKLQDVHNFK | PTH(1-35)a, | 68 |
| (γE-COC16H32CO2H)-NH2 | E13, K35(γE- | |
| | diacidC18) | |
| SVSEIQLMHNLGKHLNSMEAibVEWLRKKLQDVHNFK | PTH(1-35)a, | 69 |
| (γE-COC16H32CO2H)-NH2 | Aib20, K35(γE- | |
| | diacidC18) | |
| SVSEIQLMHNLGKHLNAibMERVEWLRKKLQDVHNFK | PTH(1-35)a, | 70 |
| (γE-COC16H32CO2H)-NH2 | Aib17, K35(γE- | |
| | diacidC18) | |
| SVSEIQLMHNLGKHLAibSMERVEWLRKKLQDVHNFK | PTH(1-35)a, | 71 |
| (γE-COC16H32CO2H)-NH2 | Aib16, K35(γE- | |
| | diacidC18) | |
| SVSEIQLMHNLAibKHLNSMERVEWLRKKLQDVHNFK | PTH(1-35)a, | 72 |
| (γE-COC16H32CO2H)-NH2 | Aib12, K35(γE- | |
| | diacidC18) | |
| SVSEIQLMHNLGEHLNSMERVEWLRERLQDVHK | PTH(1-33), | 74 |
| (γE-COC16H32CO2H)-OH | E13, E26, R27, | |
| | K33(γE- | |
| | diacidC18) | |
| SVSEIQLMHNLGEHLNSMERVEWLREKLQDVHKF | PTH(1-34), | 73 |
| (γE-COC16H32CO2H)F-OH | E13, E26, | |
| | K33(γE- | |
| | diacidC18) | |
| SVSEIQLMHNLGKHLNSMERVEWLRKKLQDVHK | PTH(1-33), | 74 |
| (γE-COC16H32CO2H)-OH | K33(γE- | |
| | diacidC18) | |
| SVSEIQLMHNLGKHLNSMERVEWLRK | PTH(1-33), | 75 |
| (γE-COC16H32CO2H)KLQDVHQ-OH | K26(γE- | |
| | diacidC18), Q33 | |
| SVSEIQLMHNLGKHLNSMERVEWLRKKLQDVHK | PTH(1-33), | 76 |
| ((γE)3-COC16H32CO2H)-OH | K33((3xγE- | |
| | diacidC18) | |
| SVSEIQLMHNLGKHLNSMERVEWLRKKLQDVHK | PTH(1-33), | 77 |
| (γE-(miniPEG)2-γE-COC16H32CO2H)-OH | K33(γE- | |
| | 2xOEG-γE- | |
| | diacidC18) | |
| kGSVSEIQLMHNLGKHLNSMERVEWLRKKLQDVHK | PTH(1-33), | 78 |
| (γE-(miniPEG)2-γE-COC16H32CO2H)-OH | dK-1, G0, K33(γE- | |
| | 2xOEG-γE-diacidC18) | |
| k(γE-(miniPEG)2-γE-COC16H32CO2H)G | PTH(1-33), dK- | 79 |
| SVSEIQLMHNLGKHLNSMERVEWLRKKLQDVHK | 1(γE-2xOEG- | |
| (γE-(miniPEG)2-γE-COC16H32CO2H)-OH | γE-diacidC18), | |
| | G0, K33(γE- | |
| | 2xOEG-γE-diacidC18) | |
| k(N-Me) | PTH(1-33), dK- | 80 |
| GSVSEIQLMHNLGKHLNSMERVEWLRKKLQDVHK | 1, N(Me)G0, | |
| (γE-(miniPEG)2-γE-COC16H32CO2H)-OH | K33(γE- | |
| | 2xOEG-γE-diacidC18) | |
| k(N-iPr) | PTH(1-33), dK- | 81 |
| GSVSEIQLMHNLGKHLNSMERVEWLRKKLQDVHK | 1, N(iPr)G0, | |
| (γE-(miniPEG)2-γE-COC16H32CO2H)-OH | K33(γE- | |
| | 2xOEG-γE-diacidC18) | |
| Aib(N-Me)G | PTH(1-33), | 82 |
| SVSEIQLMHNLGKHLNSMERVEWLRKKLQDVHK | Aib-1, | |
| (γE-(miniPEG)2-γE-COC16H32CO2H)-OH | N(Me)G0, | |
| | K33(γE- | |
| | 2xOEG-γE- | |
| | diacidC18) | |
| SVSEIQLMHNLGKHLNSMERVEWLRKKLQDVHK | PTH(1-33), | 83 |
| (K(γE-(miniPEG)2-γE-COC16H32CO2H)2)-OH | K33(K(γE-2xOEG-γE- | |
| | diacidC18)2) | |
| kGSVSEIQLMHNLGKHLNSMERVEWLRKKLQDVHK | PTH(1-33), | 84 |
| (γE-(miniPEG)2-γE-COC18H36CO2H)-OH | dK-1, G0, | |
| | K33(γE-2xOEG-γE- | |
| | diacidC20) | |
| k(γE-(miniPEG)2-γE-COC16H32CO2H)(N-Me)G | PTH(1-33), dK- | 85 |
| SVSEIQLMHNLGKHLNSMERVEWLRKKLQDVHK | 1(γE-2xOEG- | |
| (COCH3)-OH | γE-diacidC18), | |
| | N(Me)G0, K33(Ac) | |
| k(γE-(miniPEG)2-γE-COC16H32CO2H)(N-Me)G | PTH(1-33), dK- | 86 |
| SVSEIQLMHNLGKHLNSMERVEWLRKKLQDVHK | 1(γE-2xOEG- | |
| (γE-COC15H31)-OH | γE-diacidC18), | |
| | N(Me)G0, K33(γE- | |
| | monoacidC16) | |
| k(γE-(miniPEG)2-γE-COC16H32CO2H)(N- | PTH(1-33), dK- | 87 |
| Me)GSVSEIQLMHNLGKHLNSMERVEWLRKKLQDVHK( | 1(γE-2xOEG- | |
| γE-(miniPEG)2-γE-COC16H32CO2H)-OH | γE-diacidC18), | |
| | N(Me)G0, K33(γE- | |
| | 2xOEG-γE- | |
| | diacidC18) | |
| SVSEIQAMHNLGKHLNSMERVEWLRKKLQDVHK-OH | PTH(1-33), A7, K33 | 88 |
| SVSEIQLAHNLGKHLNSMERVEWLRKKLQDVHK-OH | PTH(1-33), A8, K33 | 89 |
| SVSEIQLMHNLGKHLNSMEAVEWLRKKLQDVHK-OH | PTH(1-33), | 90 |
| | A20, K33 | |
| SVSEIQLMHNLGKHLNSMERVEALRKKLQDVHK-OH | PTH(1-33), | 91 |
| | A23, K33 | |
| SVSEIQLMHNLGKHLNSMERVEWARKKLQDVHK-OH | PTH(1-33), | 92 |
| | A24, K33 | |
| SVSEIQLAHNLGKHLNSMERVEWLRKKLQDVHK | PTH(1-33), A8, | 93 |
| (γE-(miniPEG)2-γE-COC16H32CO2H)-OH | K33(γE-2xOEG-γE- | |
| | diacidC18) | |
| k(N- | PTH(1-33), dK- | 94 |
| Me)GSVSEIQLAHNLGKHLNSMERVEWLRKKLQDVHK | 1, N(Me)G0, | |
| (γE-(miniPEG)2-γE-COC16H32CO2H)-OH | A8, K33(γE-2xOEG-γE- | |
| | diacidC18) | |
| k(γE-(miniPEG)2-γE-COC16H32CO2H)(N-Me)G | PTH(1-33), dK- | 95 |
| SVSEIQLAHNLGKHLNSMERVEWLRKKLQDVHK | 1(γE-2xOEG- | |
| (γE-(miniPEG)2-γE-COC16H32CO2H)-OH | γE-diacidC18), | |
| | N(Me)G0, A8, | |
| | K33(γE-2xOEG-γE- | |
| | diacidC18) | |

### Example 3: Assessment of Cleavage Half-Life

PTH prodrugs were dissolved in PBS buffer and adjusted to obtain a pH of 7.4. The resulting solution was incubated at 37° C. Aliquots were taken at designed time points and analyzed by LC-MS. The analysis was performed using an Agilent 1260 Infinity instrument with Phenomenex Kinetex C8 2.6μ 100 A (75×4.6 mm) column. Flow rate of 1 mL/min and a gradient of 10%-80% acetonitrile in water, with 0.1% trifluoroacetic acid over 10 min. Data was collected using absorption at 214 nm. Positive mode MS data were obtained with an Agilent 6120 Quadrupole LC/MS. The concentration of prodrug and drug were determined by their relative peak areas. The zero order dissociation rate constant of the prodrug was determined by plotting the logarithm in the concentration of the prodrug at various time points. The slope of this plot provides the rate constant ‘k’. The half-life of the cleavage was them calculated based upon the formula t_{1/2}=0.693/k. Table 3 presents the cleavage half-lives of various embodiment of the PTH conjugates of the present disclosure.

TABLE 3
Dipeptide-SVSEIQLMHNLGKHLNSMERVEWLRKKLQDVHNF
→ DKP + Active peptide

| Peptides | Prodrug Dipeptide | Half life | |
|---|---|---|---|
| SEQ ID | dK(γE-diacidC18), N(Me)G | 90 h | |
| NO: 45 | PTH-peptide | | |
| SEQ ID | K(γE-diacidC18), N(Me)G | 130 h | 1.4 slower than dK |
| NO: 52 | PTH-peptide | | (vs SEQ ID NO: 45) |
| SEQ ID | dK(γE-diacidC18), N(iPr)G | 47 h | 1.9 faster than Sar |
| NO: 46 | PTH-peptide | | (vs SEQ ID NO: 45) |
| SEQ ID | K(γE-diacidC18), N(iPr)G | 67 h | 1.9 faster than Sar |
| NO: 48 | PTH-peptide | | (vs SEQ ID NO: 52) & |
| | | | 1.4 slower than dK |
| | | | (vs SEQ ID NO: 46) |
| SEQ ID | K(γE-diacidC18), N(Me)A | 71 h | 1.8 faster than Sar |
| NO: 53 | PTH-peptide | | (vs SEQ ID NO: 52) |
| SEQ ID | K(γE-diacidC18), Pip | 250 h | 1.9 slower than Sar |
| NO: 54 | PTH-peptide | | (vs SEQ ID NO: 52) |
| SEQ ID | dK, N(Me)G, PTH | 49 h | 1.8 faster without |
| NO: 57 | K35(γE-diacidC18) | | dipeptide fatty acid |
| | | | (vs SEQ ID NO: 45) |
| SEQ ID | dK(γE-diacidC18), N(Me)G, | 73 h | 1.5 slower with |
| NO: 58 | PTH K³⁵(γE-diacidC18) | | dipeptide fatty acid |
| | | | (vs SEQ ID NO: 57) |

### Example 4: Bioassay Experimental Design: Luciferase-Based Reporter Gene Assay for cAMP Detection

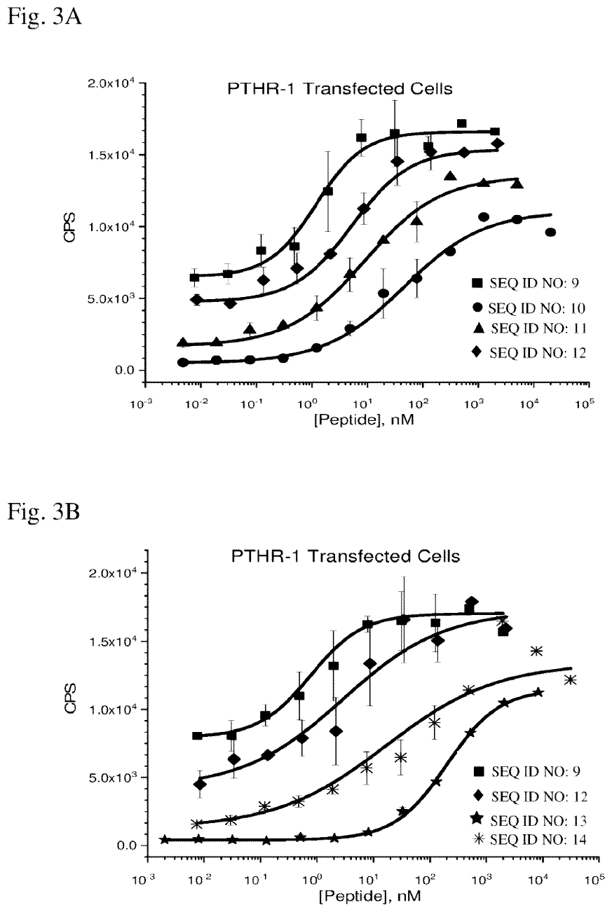

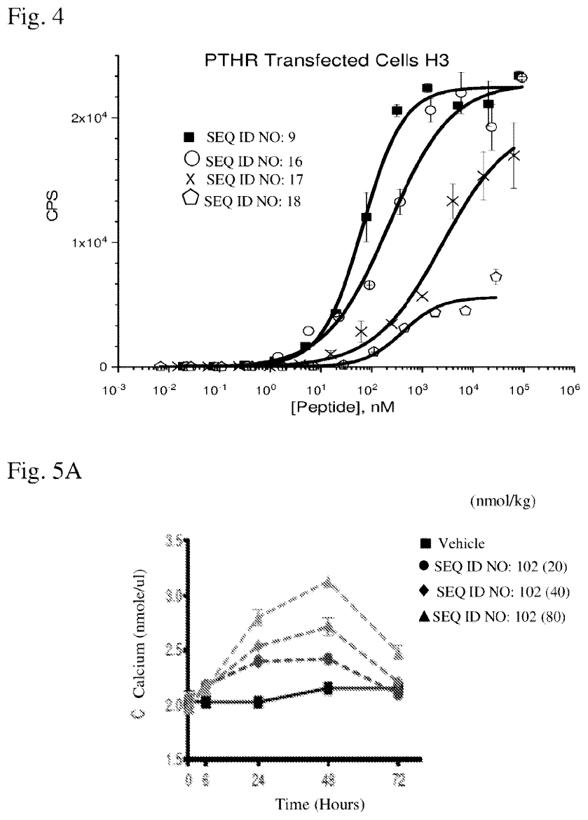

The ability of each PTH analog or prodrug to induce cAMP was measured in a firefly luciferase-based reporter assay. The cAMP production that is induced is directly proportional to the PTH binding to its receptor. HEK293 cells co-transfected with the PTH1 receptor and luciferase gene linked to a cAMP responsive element were employed for the bioassay. Results are shown in FIGS. 3A, 3B, 4 and Table 4.

The cells were serum-deprived by culturing 16 hours in Dulbecco Minimum Essential Medium (Gibco, Life Technologies, Grand Island, NY) supplemented with 0.3% FetalClone III (HyClone, Logan, UT) and then incubated with serial dilutions of PTH analogs or prodrugs for 5 hours at 37° C., 5% CO₂ in 96-well “Costar 3610” Assay plates (Corning, Kennebunk, ME). At the end of the incubation, 50 μL of Steady-Lite Plus (PerkinElmer, Waltham, MA) were added to each well. The plate was shaken briefly, incubated four minutes and light output was measured on an EnSpire Alpha Multi-mode Plate Reader (PerkinElmer, Waltham, MA). The effective 50% concentrations (EC50) were calculated using Origin 2019b software (OriginLab, Northampton, MA).

TABLE 4
In vitro analysis of PTH analogs in drug and prodrug form

| | Time | Therapeutic | Potency | | Form of |
|---|---|---|---|---|---|
| Peptide | action | Indent | EC₅₀ | | Control |
| SEQ ID NO: 32; | | drug | 1.2 | nM | |
| PTH(1-34) | | | | | |
| SEQ ID NO: 56: | protracted | drug | 2.9 | nM | Kₑₗᵢₘ |
| PTH(1-35)a, | | | | | |
| K35(γE-diacidC18) | | | | | |
| SEQ ID NOs: 45, | | prodrug | 45.5 | nM | K_{conv} |
| 36 and 61 | | | | | |
| PTH(1-34) dK- | | | | | |
| 1(γE-diacidC18), | | | | | |
| N(Me)G0 | | | | | |
| SEQ ID NO: 58: | protracted | prodrug | 200.0 | nM | K_{elim & conv} |
| PTH(1-35)a, dK- | | | | | |
| 1(γE-diacidC18), | | | | | |
| N(Me)G0, K35(γE- | | | | | |
| diacidC18) | | | | | |

### Example 5: Assessment of Pharmacology in Normal Rats

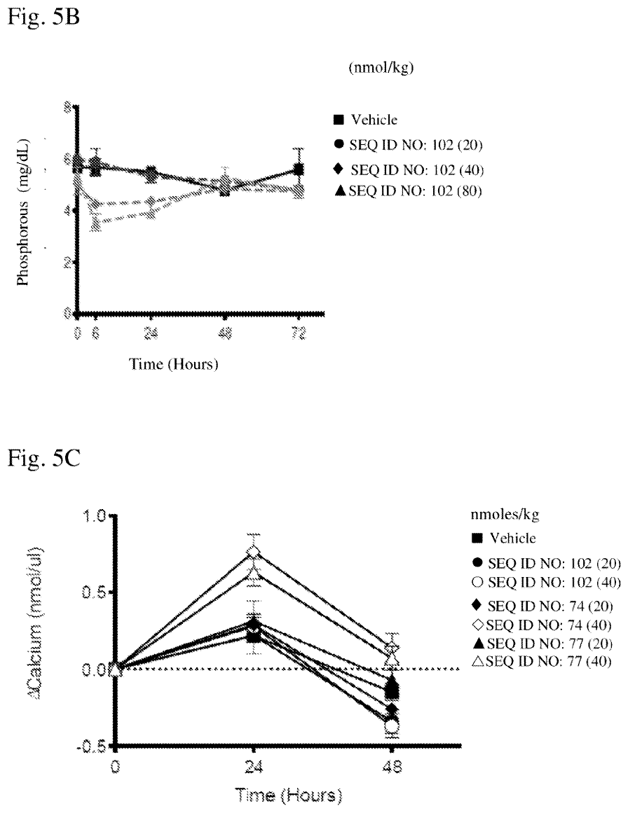

Vehicle and compound SEQ ID NO: 102 (at doses of 20, 40 & 80 nmol/kg) were injected subcutaneously in four groups of six rats (Sex: female; Strain: Sprague Dawley; Average Body Weight: 267.3 g; Age: 20-22 week-old; Diet: standard chow). Each group of rats was bled immediately before the injection and after 6, 24, 48 and 72 hours. Blood was spun and serum was collected and stored at −20° C. Calcium and phosphorous concentrations were determined used commercially available assays following the manufacturer's instructions. Results are shown in FIGS. 5A and 5B.

### Example 6: Assessment of Pharmacology in Normal Mice

Vehicle and compounds SEQ ID NO: 102, SEQ ID NO: 74 and SEQ ID NO: 77 (each at doses of 20 or 40 nmol/kg) were injected subcutaneously in seven groups of eight mice (Sex: Male; Strain: C57Bl6/J; Average Body Weight: 24.8 g; Age: 8-10 week of age; Diet: standard chow). Each group of mice was bled immediately before the injection and after 24 and 48 hours. Blood was centrifuges and serum was collected and stored at −20 C. Calcium concentrations were determined used commercially available assays following the manufacturer's instructions. Results are shown in FIG. 5C demonstrating the in vivo efficacy in mice for PTH analogs comprising an acylated amino acid at the C-terminus of the PTH peptide to reduce serum calcium levels.

### Example 7: Assessment of Pharmacokinetics in Normal Mice—Measuring Plasma Peptide Concentration by LCMS

Compounds SEQ ID NO: 78, SEQ ID NO: 79 and SEQ ID NO: 84 were injected subcutaneously at a dose of 100 nmol/kg in mice (Sex: Male; Strain: C57Bl6/J; Average Body Weight: 34.8 g; Age: 42-44-week-old; Diet: standard chow). Each compound was injected in 20 mice and blood was collected on EDTA-coated tubes from 4 mice from each treatment at times: 1, 4, 8, 24 & 48 hours. Plasma was collected after centrifugation and stored at −20° C.

Standard curve samples were prepared by serial dilution with cd-1 mouse plasma on the day of analysis. Aliquots (40 μl) of standard curve and study samples were transferred to a 96-well plate and mixed with 160 μl of Methanol:Acetonitrile (ACN) (1:1, v/v) internal standard solution. After 10 minutes of centrifugation, supernatants were diluted 2-fold with acidified (0.1% formic acid) ACN:Water (3:1, v/v) and analyzed by LC-MS/MS.

A Shimadzu CBM-20A Nexera UPLC system and a CTC PAL autosampler comprised the front end of the LC-MS/MS system. Chromatography was based on an Accurcore C8 Column, 2.6 μm, 2.1 mm×30 mm (Thermo 17226-032130) and a binary gradient program of 0.1% formic acid (aq) and 0.1% formic acid in ACN. Mobile phase solvent A consisted of micro filtered water:formic acid (1000:1 v/v), and solvent B consisted of ACN:formic acid (1000:1). The flow rate was 0.8 ml/min., the column temperature was ambient, and the injection volume was 5 μl. The two-needle rinses were ACN:Water (25:75, v/v) and ACN:Isopropanol:Acetone in 0.1% formic acid (5:4:1, v/v/v). The gradient cycle started at 15% B (concentration) with a linear increase to 65% B in 0.75 minute. The column was cleaned with 98% B for 0.25 min. and returned to initial % B during acquisition time of 1.20 minute. The total cycle time for each injection, including re-equilibration of initial % B, was approximately 3 minutes. The first 0.3 min. of each run was diverted to waste.

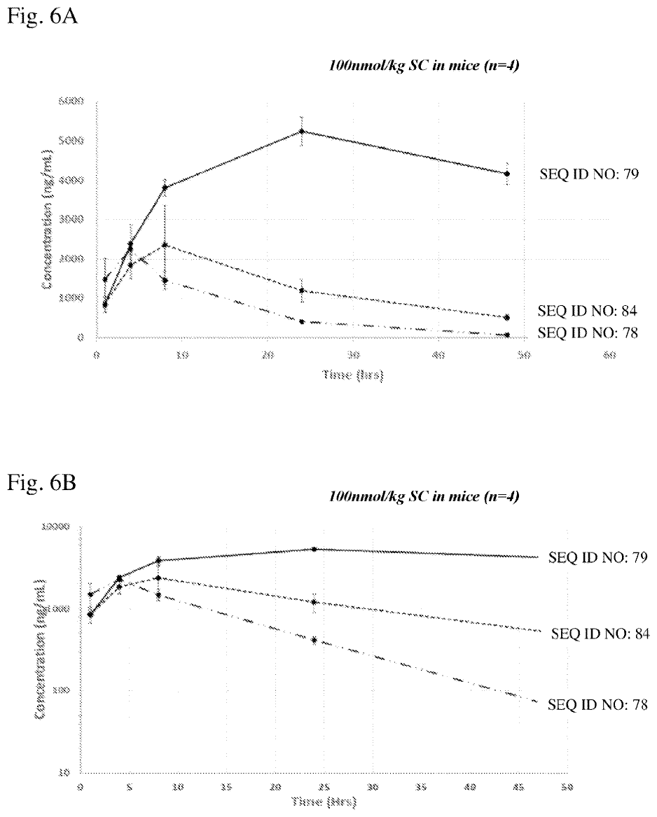

Mass spectrometric data were generated using Analyst software controlling a Sciex API 6500 plus triple-quadrupole mass spectrometer (Model 5060743-J) in positive ionization mode. The multiple reaction monitoring (MRM), collision energies, declustering potential and collision exit potential settings for each test article and the internal standard (IS) are provided in Table 5. Concentrations of each analog over time are shown in FIGS. 6A and 6B.

TABLE 5
Mass Spectural Assessment of PTH Analogs

| | | Collision | | |
|---|---|---|---|---|
| | | Energy | Declustering | Collision Exit |
| Test Article | MRM | (eV) | Potential | Potential |
| SEQ ID NO: 78 | m/z 836.8→ m/z 910.2 | 40 | 125 | 6 |
| SEQ ID NO: 79 | m/z 977.6→ m/z 974.7 | 38 | 125 | 6 |
| SEQ ID NO: 84 | m/z 841.6→ m/z 910.3 | 40 | 125 | 6 |
| Imipramine (IS) | m/z 281.2→ m/z 58.1 | 55 | 50 | 15 |

### Example 8: PTH Analogues: Comparative Pharmacokinetics in Cynomolgus Monkeys Following a Single Subcutaneous Dose

The pharmacokinetic profile of PTH analogues following a single subcutaneous dose to cynomolgus monkeys and an evaluation of the pharmacodynamic response of serum calcium and phosphorus was investigated. A total of six monkey were used in this test 2 males and 4 females ranging in age from 2-4 years old at beginning of the study and having a weight of at least 2.5 kg.

### Experimental Design: Route, Frequency, and Duration of Administration

Animals were assigned to groups as noted in the Table 6 below. Animals were dosed by subcutaneous injection. Dose administration was staggered. The Group 1

TABLE 6

| | | Nominal | Nominal | | | |
|---|---|---|---|---|---|---|
| | | Dose | Dose Con- | Dose | Number | Number |
| | | Level | centration | Volume | of | of |
| Group | Treatment | (nmol/kg) | (mg/ml) | (mL/kg) | males | females |
| 1 | SEQ ID | 25 | 0.48 | 0.3 | 1 | 2 |
| | NO: 95 | | | | | |
| 2 | SEQ ID | 25 | 0.41 | 0.3 | 1 | 2 |
| | NO: 94 | | | | | |

male was dosed once, and the remaining two Group 1 females and all Group 2 animals were dosed once, approximately four days later. First day of dosing was designated as Study Day (SD) 1 for each animal. The dose volume was 0.3 mL/kg. Individual dose volumes were calculated based on the animals' most recently recorded body weight. Animals were observed and data was recorded as indicated in Table 7.

TABLE 7
Observations of Animals

| Procedure | Frequency |
|---|---|
| Cage-side Observations | At least twice daily |
| (mortality, moribundity, and general | |
| health) | |
| Physical Examinations | Prior to dosing on SD 1 and daily |
| (skin and fur characteristics, eye and | thereafter with unscheduled |
| mucous membranes, respiratory, | observations recorded. |
| circulatory, autonomic and central | |
| nervous systems, somatomotor and | |
| behavior patterns) | |
| Body Weights | Prior to dosing on SD 1 and |
| | on SD 4 |

At least a 0.5 mL sample of blood was collected from all animals at predose, and at 1, 2, 4, 8, 24, 48, 72, 96, 120-, 144-, 168- and 192-hours following dosing. Animals were not fasted prior to collection. Blood was be collected via the femoral vein (or another appropriate site). Blood samples were maintained at 5±3° C. (wet ice or equivalent) and centrifuged at 5±3° C. within 1 hour of the collection of each blood sample. The resultant plasma was transferred to a tube and then stored under conditions set to maintain −75±15° C. until analysis.

### Pharmacokinetic Evaluation

Pharmacokinetic mean concentration-time data was analyzed using non-compartmental methods (Phoenix® WinNonlin® Version 7.0 or higher) based on the route of administration. The following parameters were calculated whenever possible and as data allows: Cmax, Tmax, and AUC. Descriptive statistics were generated using Phoenix WinNonlin.

### Results

To minimize mortality in the monkey study while evaluating the pharmacokinetics of the PTH analogs, a low potency alanine substituted PTH agonist SEQ ID NO: 93 PTH(1-33), A8, K33(γE-2×OEG-γE-diacidC18) was studied in two prodrug forms of it: SEQ ID NO: 94 PTH(1-33), A8, K33(γE-2×OEG-γE-diacidC18) with dK-1, N(Me)G0 and SEQ ID NO: 95 PTH(1-33), A8, K33(γE-2×OEG-γE-diacidC18) with dK-1(γE-2×OEG-γE-diacidC18), N(Me)G0 were administered to the monkeys. Substitution at position 8 with alanine reduces in vitro potency by nearly 100-fold.

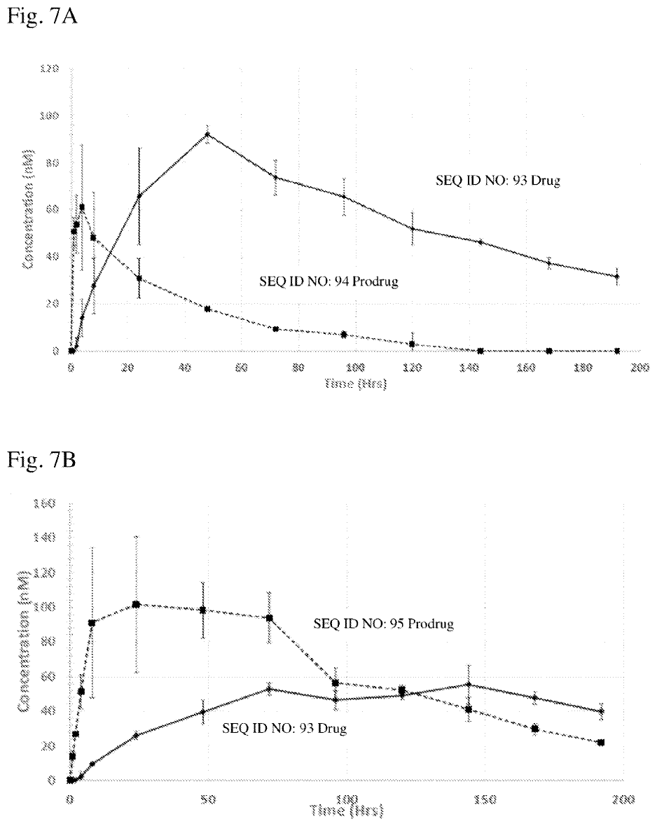

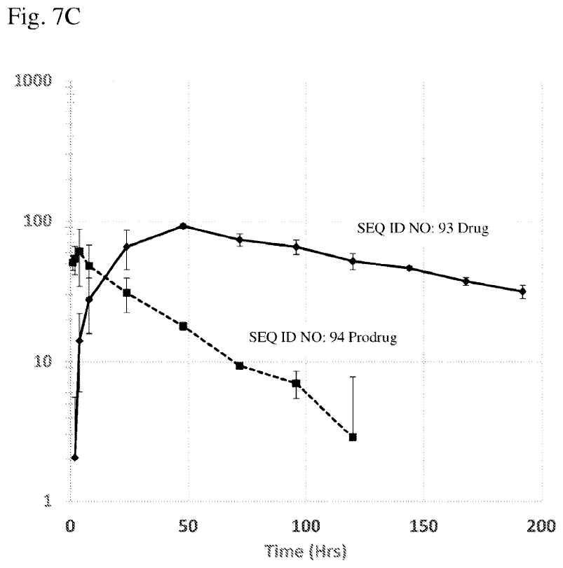

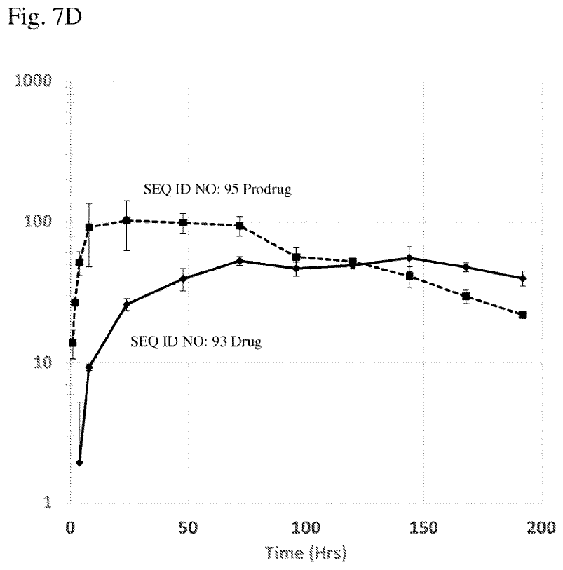

Results from these pharmacokinetic (PK) studies are provided in FIGS. 7A and 7B wherein the concentration of the administered prodrug form and its activate drug form (produced after in vivo non-enzymatic cleavage of the dipeptide prodrug element) were measured over time. Monkeys were administered a single subcutaneous dose of a prodrug PTH-analog at 25 nmol/kg (SEQ ID NO: 94 in FIG. 7A and Peptide 19 in FIG. 7B) and the concentration of the prodrugs SEQ ID NO: 94 and SEQ ID NO: 95 along with their corresponding cleavage product, SEQ ID NO: 93 (“the drug”) were measured over the next 192 hours after administration.

Specifically, FIG. 7A is a graph demonstrating the detected levels of the prodrug PTH analog SEQ ID NO: 94 PTH(1-33), A8, K33(γE-2×OEG-γE-diacidC18) dK⁻¹, N(Me)G⁰; and its activated form SEQ ID NO: 93 PTH(1-33), A8, K33(γE-2×OEG-γE-diacidC18) over time after administration of a single dose of the prodrug (SEQ ID NO: 94). FIG. 7B is a graph demonstrating the detected levels of the prodrug PTH analog SEQ ID NO: 95 PTH(1-33), A8, K33(γE-2×OEG-γE-diacidC18) dK⁻¹(γE-2×OEG-γE-diacidC18), N(Me)G⁰ and its activated form: SEQ ID NO: 93 PTH(1-33), A8, K33(γE-2×OEG-γE-diacidC18) over time after administration of a single dose of the prodrug (SEQ ID NO: 95). The data demonstrate accumulation of the active form over time, corresponding with a decrease of the prodrug form, resulting in a relatively consistent amount of active form over an extended time-period.

### Example 9: Serum Calcium Levels in C57BL/6J Mice Measured 24 and 48 Hours after Subcutaneous Administration of 20 or 40 nM/kg of SEQ ID NO: 77

Female C57BL/6J mice were randomly assigned to three groups (n=8 per group) and injected subcutaneously once with either vehicle (sterile PBS solution) or SEQ ID NO: 77 in PBS (20 or 40 nmol/kg). SEQ ID NO:77 was prepared at a concentration of 150 μM in PBS, pH 7.4. Blood samples were collected from each mouse via tail laceration for serum calcium determination just prior to administration (0 hours) and post administration (24 and 48 hours).

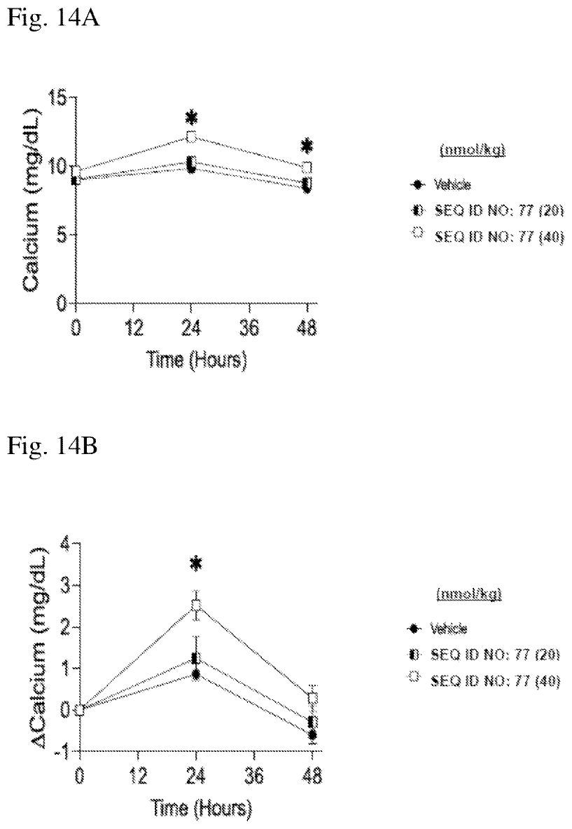

The serum Ca concentrations were determined using a commercially available colorimetric assay following the manufacturer's recommendations (Calcium LiquiColor #10155, Stanbio Laboratory). The Ca assay contains a color reagent (Cat. No. 0156) and a Ca Standard (10 mg/dl; Cat. No. 0157). Absorbance (Ab) was measured at 650 nm wavelength. Manufacturer linearity is provided up to 15 mg/dl. Results were calculated as follows: Ab(unknown)/Ab(standard)×10 Serum calcium levels were recorded for control and treatment groups at time zero, 24 and 48 hrs., and are summarized in Tables 8-10. Mice receiving a single subcutaneous dose of SEQ ID NO: 77 at 40 nmol/kg exhibited significantly increased serum Ca levels at 24 and 48 hours post treatment compared to controls or to those receiving SEQ ID NO: 77 at 20 nmol/kg (FIG. 14A). This difference was statistically significant at 24 hours when accounting for the initial baseline levels (FIG. 14B). There was an increase in serum Ca at 24 hours following the dose of 40 nmol/kg. This increase of 0.631 nmol/uL (2.524 mg/dl) from a starting level of 2.4105 nmol/uL (9.6421 mg/dl) represents a 26.2% higher concentration.

Percentage change of serum Ca levels for each study group are summarized in Table 11. The serum Ca level increase for the 40 nmol/kg dose group was nearly double the increase (13.8%) at the lower dose of 20 nmol/kg SEQ ID NO: 77. At this dose there was an increase of 0.3131 nmol/ul (1.252 mg/dl), starting from a level of 2.2704 nmol/ul (9.0816 mg/dl). The increase in serum Ca upon vehicle treatment was less than either group treated with SEQ ID NO: 77. The serum Ca increase for the vehicle group animals of 9.7% represents a value of 0.2174 nmol/uL (0.898 mg/dl), starting from a level of 2.2512 nmol/uL (9.0049 mg/dl). In all treatments, the serum Ca returned to near starting levels at 48 hours post treatment.

TABLE 8
Serum Calcium Levels at time point 0 hour

| | | Time | | | Conc. |
|---|---|---|---|---|---|
| Group | ID | (Hrs) | A650 | Conc.(mg/dl) | (nmol/uL) |
| Group | 9 | 0 | 0.2381 | 8.6945 | 2.1736 |
| C | 10 | 0 | 0.2395 | 8.7457 | 2.1864 |
| | 11 | 0 | 0.2447 | 8.9355 | 2.2339 |
| | 12 | 0 | 0.2561 | 9.3518 | 2.3380 |
| | 21 | 0 | 0.2577 | 9.4103 | 2.3526 |
| | 22 | 0 | 0.2563 | 9.3591 | 2.3398 |
| | 23 | 0 | 0.2435 | 8.8917 | 2.2229 |
| | 24 | 0 | 0.2369 | 8.6507 | 2.1627 |
| Group | 41 | 0 | 0.2451 | 8.9502 | 2.2375 |
| F | 42 | 0 | 0.2428 | 8.8662 | 2.2165 |
| | 43 | 0 | 0.2569 | 9.3810 | 2.3453 |
| | 44 | 0 | 0.2568 | 9.3774 | 2.3443 |
| | 45 | 0 | 0.2797 | 10.2136 | 2.5534 |
| | 46 | 0 | 0.2643 | 9.6513 | 2.4128 |
| | 47 | 0 | 0.2387 | 8.7165 | 2.1791 |
| | 48 | 0 | 0.2053 | 7.4968 | 1.8742 |
| Group | 49 | 0 | 0.2592 | 9.4650 | 2.3663 |
| G | 50 | 0 | 0.2747 | 10.0310 | 2.5078 |
| | 51 | 0 | 0.2714 | 9.9105 | 2.4776 |
| | 52 | 0 | 0.2428 | 8.8662 | 2.2165 |
| | 53 | 0 | 0.2776 | 10.1369 | 2.5342 |
| | 54 | 0 | 0.2903 | 10.6007 | 2.6502 |
| | 55 | 0 | 0.2629 | 9.6001 | 2.4000 |
| | 56 | 0 | 0.2335 | 8.5266 | 2.1316 |

TABLE 9
Serum Calcium Levels at time point 24 hours

| | | Time | | | Conc. |
|---|---|---|---|---|---|
| Group | ID | (Hrs) | A650 | Conc.(mg/dl) | (nmol/uL) |
| Group | 9 | 24 | 0.2743 | 9.6144 | 2.4036 |
| C | 10 | 24 | 0.2933 | 10.2804 | 2.5701 |
| | 11 | 24 | 0.2788 | 9.7722 | 2.4430 |
| | 12 | 24 | 0.2906 | 10.1858 | 2.5464 |
| | 21 | 24 | 0.2770 | 9.7091 | 2.4273 |
| | 22 | 24 | 0.2870 | 10.0596 | 2.5149 |
| | 23 | 24 | 0.2784 | 9.7581 | 2.4395 |
| | 24 | 24 | 0.2743 | 9.6144 | 2.4036 |
| Group | 41 | 24 | 0.2616 | 9.1693 | 2.2923 |
| F | 42 | 24 | 0.2710 | 9.4988 | 2.3747 |
| | 43 | 24 | 0.2808 | 9.8423 | 2.4606 |
| | 44 | 24 | 0.3273 | 11.4721 | 2.8680 |
| | 45 | 24 | 0.3127 | 10.9604 | 2.7401 |
| | 46 | 24 | 0.2989 | 10.4767 | 2.6192 |
| | 47 | 24 | 0.2598 | 9.1062 | 2.2766 |
| | 48 | 24 | 0.3465 | 12.1451 | 3.0363 |
| Group | 49 | 24 | 0.3902 | 13.6768 | 3.4192 |
| G | 50 | 24 | 0.3293 | 11.5422 | 2.8856 |
| | 51 | 24 | 0.3632 | 12.7305 | 3.1826 |
| | 52 | 24 | 0.3297 | 11.5563 | 2.8891 |
| | 53 | 24 | 0.3734 | 13.0880 | 3.2720 |
| | 54 | 24 | 0.3299 | 11.5633 | 2.8908 |
| | 55 | 24 | 0.3340 | 11.7070 | 2.9267 |
| | 56 | 24 | 0.3271 | 11.4651 | 2.8663 |

TABLE 10
Serum Calcium Levels at time point 48 hours

| | | Time | | | Conc. |
|---|---|---|---|---|---|
| | ID | (Hrs) | A650 | Conc.(mg/dl) | (nmol/uL) |
| Group | 9 | 48 | 0.2589 | 8.2742 | 2.0686 |
| C | 10 | 48 | 0.2548 | 8.1432 | 2.0358 |
| | 11 | 48 | 0.2966 | 9.4791 | 2.3698 |
| | 12 | 48 | 0.2589 | 8.2742 | 2.0686 |
| | 21 | 48 | 0.2634 | 8.4180 | 2.1045 |
| | 22 | 48 | 0.2526 | 8.0729 | 2.0182 |
| | 23 | 48 | 0.2490 | 7.9578 | 1.9895 |
| | 24 | 48 | 0.2715 | 8.6769 | 2.1692 |
| Group | 41 | 48 | 0.2763 | 8.8303 | 2.2076 |
| F | 42 | 48 | 0.2862 | 9.1467 | 2.2867 |
| | 43 | 48 | 0.2767 | 8.8431 | 2.2108 |
| | 44 | 48 | 0.2305 | 7.3666 | 1.8416 |
| | 45 | 48 | 0.2724 | 8.7057 | 2.1764 |
| | 46 | 48 | 0.3048 | 9.7411 | 2.4353 |
| | 47 | 48 | 0.2737 | 8.7472 | 2.1868 |
| | 48 | 48 | 0.2817 | 9.0029 | 2.2507 |
| Group | 49 | 48 | 0.3210 | 10.2589 | 2.5647 |
| G | 50 | 48 | 0.2840 | 9.0764 | 2.2691 |
| | 51 | 48 | 0.3148 | 10.0607 | 2.5152 |
| | 52 | 48 | 0.2896 | 9.2554 | 2.3138 |
| | 53 | 48 | 0.3128 | 9.9968 | 2.4992 |
| | 54 | 48 | 0.3111 | 9.9425 | 2.4856 |
| | 55 | 48 | 0.3322 | 10.6168 | 2.6542 |
| | 56 | 48 | 0.3205 | 10.2429 | 2.5607 |

TABLE 11
Percent change of serum Ca levels

| | mg/dl | nmol/uL | change | percent |
|---|---|---|---|---|
| Vehicle | | | | |
| 0 hr | 9.0049 | 2.2512 | | |
| 24 hr | 9.8743 | 2.4686 | 0.2174 | 9.70% |
| 48 hr | 8.412 | 2.103 | 0.1482 | |
| SEQ ID NO: 77 @ | | | | |
| 20 nmol/kg | | | | |
| 0 hr | 9.0816 | 2.2704 | | |
| 24 hr | 10.3339 | 2.5835 | 0.3131 | 13.80% |
| 48 hr | 8.7979 | 2.1995 | 0.0709 | |
| SEQ ID NO: 77 @ | | | | |
| 40 nmol/kg | | | | |
| 0 hr | 9.6421 | 2.4105 | | |
| 24 hr | 12.1661 | 3.0415 | 0.631 | 26.20% |
| 48 hr | 9.9313 | 2.4828 | 0.073 | |

### Example 10: Serum Calcium, Inorganic Phosphate, Body Weight, Food Intake and Drug Levels in Sprague Dawley Rats Following Single Individual Subcutaneous Administration of SEQ ID NO: 77 or SEQ ID NO: 87

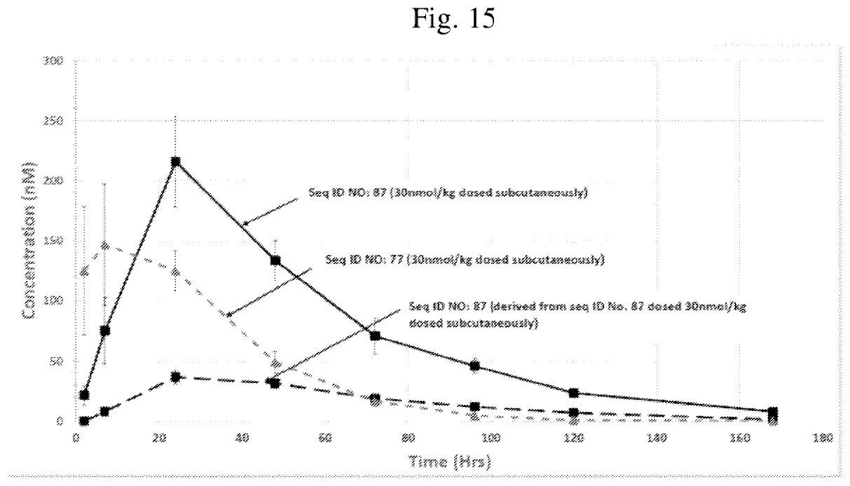

This experiment measured the profile of serum calcium and inorganic phosphorus following subcutaneous injection of 30 or 60 nmol/kg of the parathyroid hormone analogue pro-drug SEQ ID NO: 87 in female Sprague-Dawley rats. In addition, pharmacokinetics (PR) of pro-drug SEQ ID NO: 87 and its conversion to active SEQ ID NO: 77 were measured in the group receiving 30 nmol/kg SEQ ID NO: 87 and compared to a group of rats receiving 30 nmol/kg SEQ ID NO: 77. Female Sprague-Dawley rats were randomly distributed among four groups with five or six rats per group. Formulations were prepared from lyophilized powders of SEQ ID NO: 87 and SEQ ID NO: 77 and diluent, PBS, to obtain a 50 M solution of SEQ ID NO: 77 in PBS and 100 μM and 50 μM solutions of SEQ ID NO: 87 in PBS and these were maintained on wet ice during dosing. Dose volume was based on body weight and recorded prior to dosing. Each subject was injected subcutaneously once with either vehicle (sterile PBS solution; n=6), SEQ ID NO: 77 (30 nmol/kg; n=5) or SEQ ID NO: 87 (30 or 60 nmol/kg; n=5 and 6, respectively). Blood samples were collected from each rat via tail laceration just prior to administration (0 hours) and post administration (24, 48, 72, 96, 120, and 168 hours). These samples were used for serum calcium and inorganic phosphorus determination in vehicle and all SEQ ID NO: 87 treated animals. Separate blood samples were drawn for PK determinations in the SEQ ID NO: 87 and SEQ ID NO: 77 30 nmol/kg groups and additional blood samples were collected at 2 and 7 hours in these two PK groups. Data is summarized in FIGS. 15, 16A, 16B, 17A and 17B and in Tables 12 and 13.

The measurement of serum SEQ ID NO: 87 and SEQ ID NO: 77 was performed by LCMS. Pharmacokinetic results are summarized in FIG. 15. SEQ ID NO: 77 and SEQ ID NO: 87 were observed to increase in plasma concentration following a single dose at 30 nmol/kg. The prodrug form of the PTH agonist (SEQ ID NO: 87) demonstrated a Cmax at 24 h that was reduced in relative concentration by ⅓ at 48 h and ⅔ at 72 h, returning to baseline value at one week. SEQ ID NO: 77 was observed following administration of SEQ ID NO: 87 through in vivo conversion and release of the N-terminal dipeptide. It peaks at 24 h at a concentration slightly less than 20% of SEQ ID NO: 87. It was sustained to nearly the same level at 48 h and of comparable concentration to SEQ ID NO: 87 at 72 h, returning to baseline at one week. A similar PK-analysis for SEQ ID NO: 77 serum concentration following its administration at the same 30 nmol/kg dose as SEQ ID NO: 87 revealed a more rapid appearance with concentration more than 100 nM at two hours post-dosing that peaked at seven hours and was sustained through twenty-four hours but decreasing to approximately one ⅓ the Cmax at 48 h and at baseline level at 96 h. The peak to trough ratio when comparing the concentration of SEQ ID NO: 77 within the first day relative to 96 h demonstrated a difference dependent upon whether it was achieved by direct administration of the peptide or its result of conversion from SEQ ID NO: 87.

The results demonstrate that SEQ ID NO: 87 converts in vivo to SEQ ID NO: 77 at a rate that is consistent with the in vitro determinations and that the peptide is sustained for nearly a week in rats where fatty acylated peptides are accelerated in clearance relative to primates. At doses where prodrug and drug could be measured by LCMS the changes in serum calcium and phosphorous were within the physiological range and did not adversely affect the rats.

### Serum Calcium and Inorganic Phosphorus Measurements

Blood samples collected via tail laceration were used for determination of total calcium (Ca) and inorganic phosphorous (Pi) in serum using commercially available colorimetric assays following the manufacturer's recommendations (Stanbio laboratory: Calcium LiquiColor #10155; Phosphorous Liqui-UV, #0830). A 96-well plate spectrophotometer (SpectraMax M5, Molecular Devices) was employed for detection. Serum Ca and Pi were measured on days 0, 1, 2, 4, 5 and 7. Absorbance (Ab) was measured at 650 nm for Ca and 340 nm for Pi. Manufacturer linearity is provided up to 15 mg/dl for Ca and 10 mg/dl for Pi. Results were calculated as follows: Ab(unknown)/Ab(standard)×10

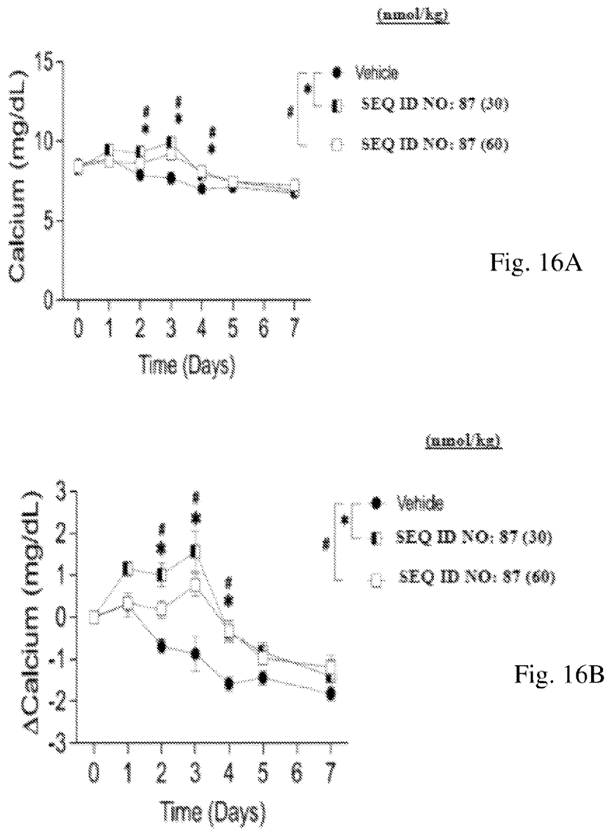

Serum calcium was observed to increase significantly relative to vehicle treatment in the time range of 48 through 96 hours, returning to levels comparable to control values at 120 and 240 hours (FIGS. 16A, 16B and Table 12). The measurement of serum calcium following administration of SEQ ID NO: 87 demonstrated an increase within the physiological range that peaked at 3 days. The lowest dose of the peptide provided a slightly enhanced increase that was not statistically significant relative to the higher dose.

TABLE 12
Serum calcium change 0 vs 72 hours

| | mg/dl | nmol/uL | change mg/dl | change nmol/ul |
|---|---|---|---|---|
| Vehicle | | | | |
| 0 hr | 8.5726 | 2.1432 | | |
| 72 hr | 7.6998 | 1.925 | 0.8728 | 0.2182 |
| 60 nmol/kg | | | | |
| 0 hr | 8.4274 | 2.1068 | | |
| 72 hr | 9.1988 | 2.2997 | 0.7714 | 0.1929 |
| 30 nmol/kg | | | | |
| 0 hr | 8.2985 | 2.0746 | | |
| 72 hr | 9.9306 | 2.4827 | 1.6321 | 0.4081 |

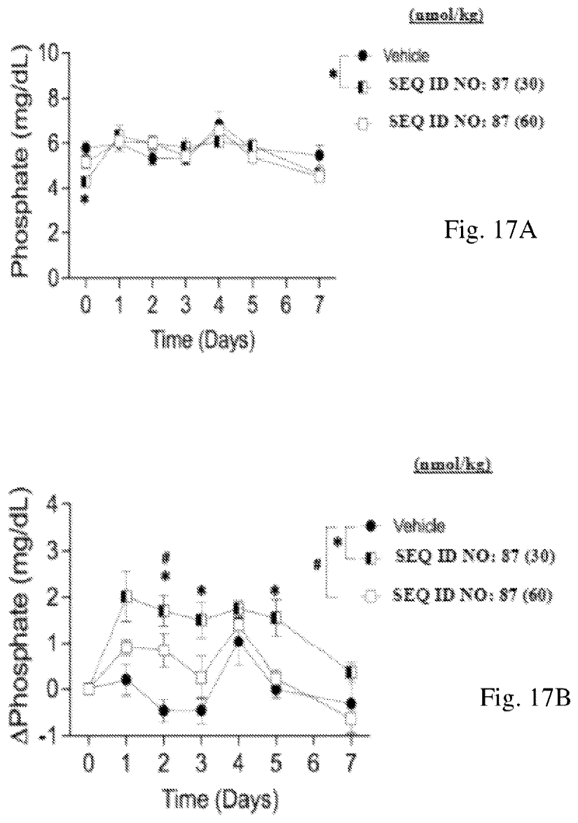

There was a smaller increase in serum phosphate levels that was statistically significant only when assessed relative to vehicle treatment. This difference was attributed to the lower starting concentration in the vehicle treated rats (FIGS. 17A, 17B and Table 13).

TABLE 13
Serum phosphate changes 0 vs 72 hours

| | mg/dl | nmol/uL | change mg/dl | change nmol/ul |
|---|---|---|---|---|
| vehicle | | | | |
| 0 hr | 8.5726 | 2.1432 | | |
| 72 hr | 7.6998 | 1.925 | 0.8728 | 0.2182 |
| 60 nmol/kg | | | | |
| 0 hr | 8.4274 | 2.1068 | | |
| 72 hr | 9.1988 | 2.2997 | 0.7714 | 0.1929 |
| 30 nmol/kg | | | | |
| 0 hr | 8.2985 | 2.0746 | | |
| 72 hr | 9.9306 | 2.4827 | 1.6321 | 0.4081 |

### Example 11: Assay of Prodrug and its Conversion to Drug by LCMS & in Vitro Assay

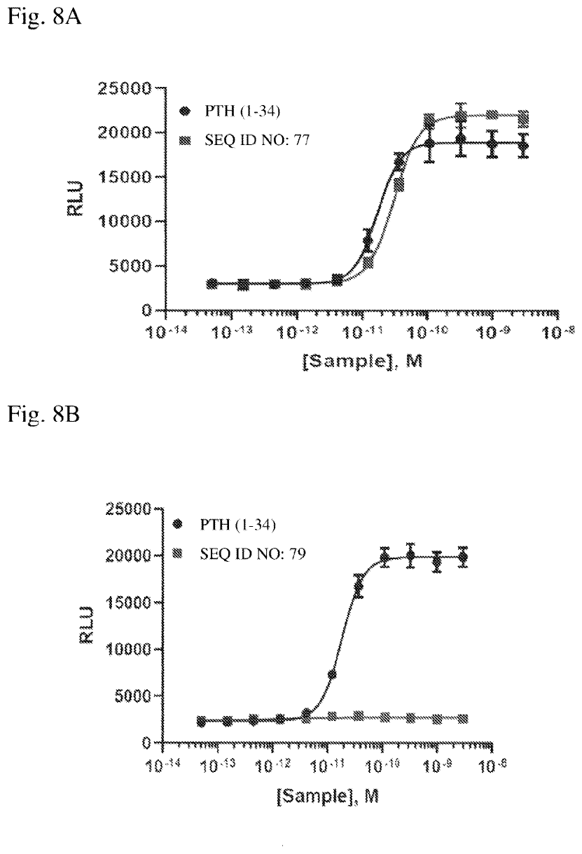

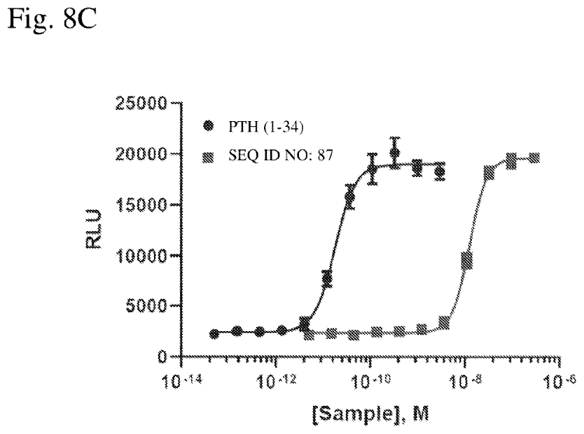

The assessment of the bioactivity of a prodrug relative to its drug form was conducted in a proprietary commercial assay as contracted with Eurofins-Discover Rx Corporation win a cAMP Hunter® Teriparatide Bioassay (95-0118Y2). An 11-point dose curve in agonist mode was conducted for each individual peptide or incubation condition. Every sample was run at each dose in triplicate. The results in Table 14 are presented in FIG. 8A-8C, and they support the conclusions that SEQ ID No 87 is a full PTH agonist of high nM potency (FIG. 8A), and that the extension of SEQ ID No. 87 with a non-cleavable dipeptide as constituted in SEQ ID No. 79 (FIG. 8B) or a dipeptide as constituted in SEQ ID No. 77 (FIG. 8C) inactivates the PTH agonism.

TABLE 14
In vitro results associated with FIGS. 8A, 8B and 8C

| Sample | S/B | Hillslope | EC₅₀ pM |
|---|---|---|---|
| SEQ ID NO: 77 | 8.0 | 2.196 | 31 |
| PTH 1-34 | 6.1 | 2.017 | 17.3 |
| SEQ ID NO: 79 | 8.0 | — | no activity |
| PTH 1-34 | 7.8 | 2.017 | 18.1 |
| SEQ ID NO: 87 | 8.0 | 2.387 | 12,730 |
| PTH 1-34 | 9.0 | 2.229 | 18.6 |

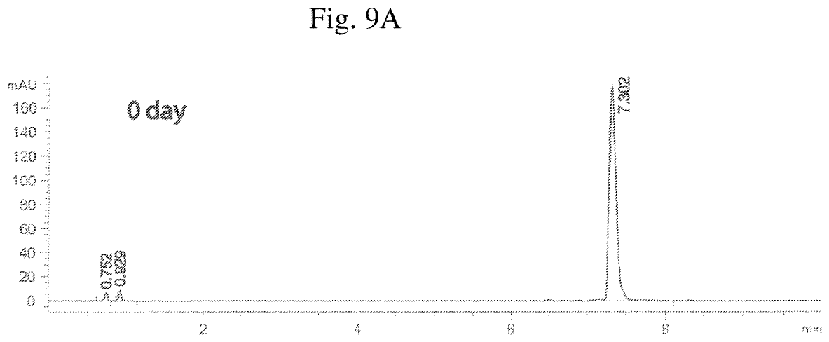

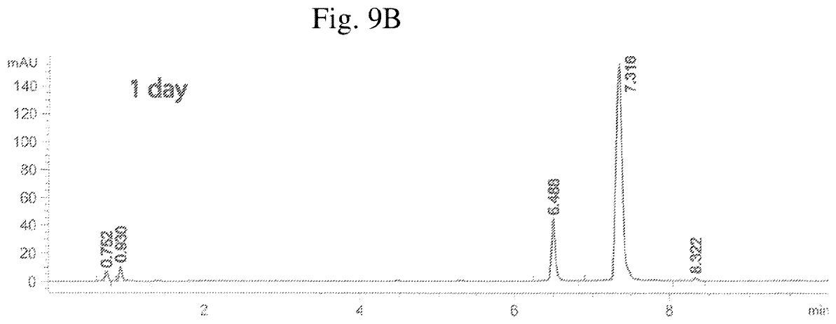

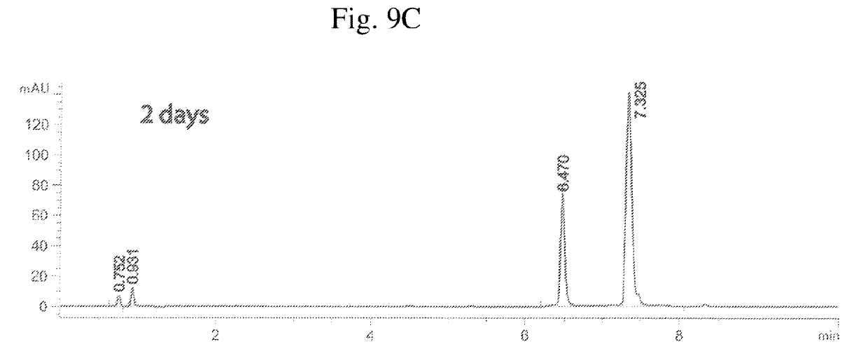

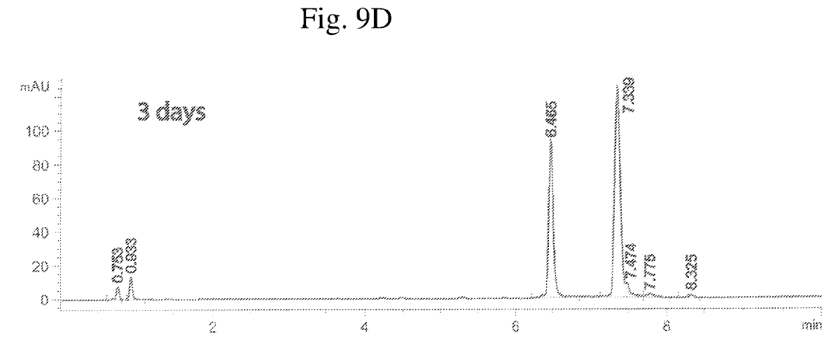

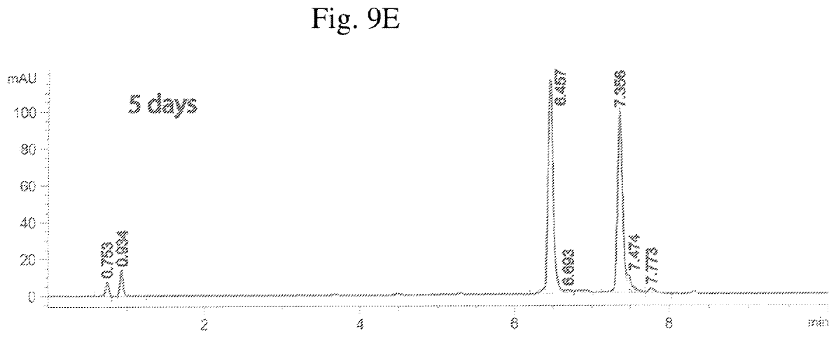

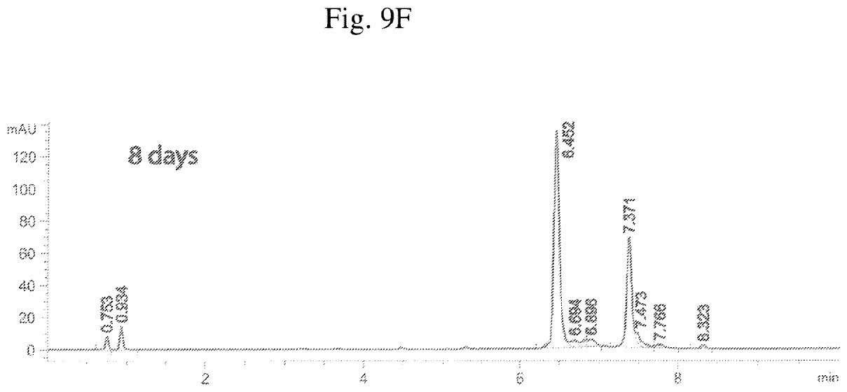

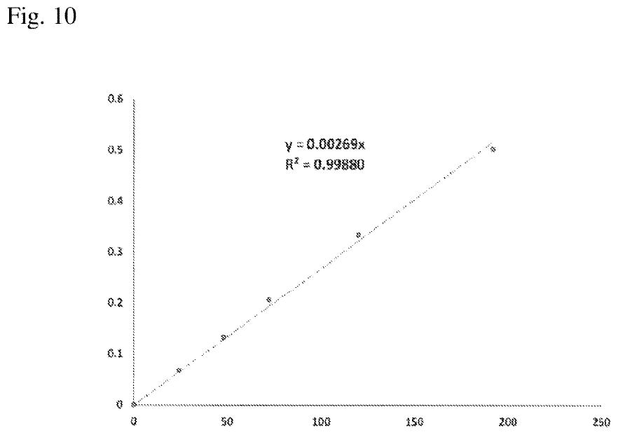

The assessment of the conversion of the prodrug to drug was determined by LC-MS analysis where the methodology is presented in Example 3. The peak areas and the calculation of the percent prodrug is shown in Table 15. The individual chromatographic spectra are shown in FIGS. 9A-9F. The calculation of the rate of reaction was performed by fitting the experimental results to a zero-order equation that demonstrates a high correlative fit with an R2 value of 0.9988 (FIG. 10).

TABLE 15
Peak area of Prodrug and Drug as shown
in FIGS. 9A-9F and 10 results

| | Peak | Peak | Total | | |
|---|---|---|---|---|---|
| | area of | area of | peak | Percentage | -log(percentage of |
| | drug | prodrug | area | of prodrug | prodrug) |
| 0 h | 0 | 1090.4 | 1090.4 | 100% | 0 |
| 24 h | 159.2 | 935.2 | 1094.4 | 85% | 0.068272 |
| 48 h | 292.4 | 813.5 | 1105.9 | 74% | 0.133358 |
| 72 h | 400.3 | 656.1 | 1056.4 | 62% | 0.206858 |
| 120 h | 550.8 | 475.9 | 1026.7 | 46% | 0.333928 |
| 192 h | 680.9 | 311.9 | 992.8 | 31% | 0.502846 |

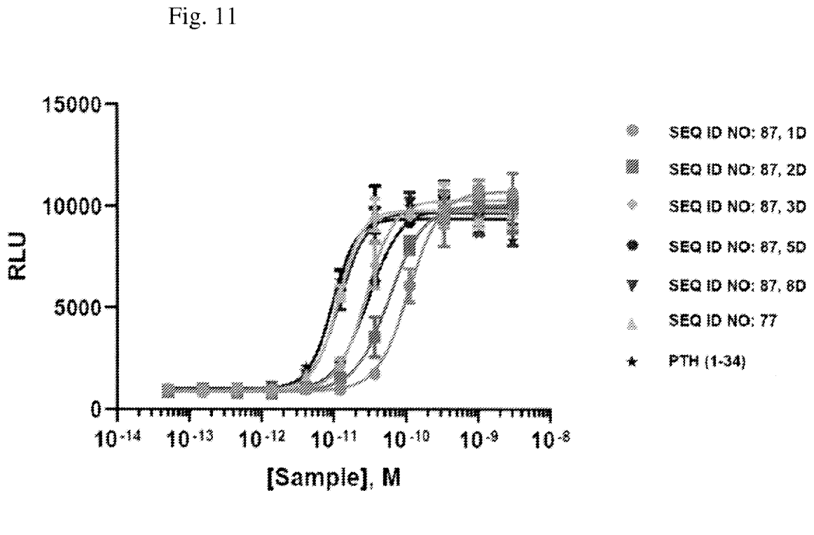

The assessment of the conversion of the prodrug to drug was additional conducted in a proprietary commercial assay as contracted with Eurofins-Discover Rx Corporation win a cAMP Hunter® Teriparatide Bioassay (95-0118Y2). An 11-point dose curve in agonist mode was conducted for each individual peptide or incubation condition. Every sample was run at each dose in triplicate. The analysis of the same incubation samples that were assessed for relative prodrug/drug concentration by LCMS (FIGS. 9A-9F, and FIG. 10) was conducted using the cAMP Hunter® Teriparatide Bioassay. The results in Table 16 are presented in FIG. 11 and they support the conclusion as determined above by LCMS.

TABLE 16
In vitro results associated with FIG. 11.

| Sample | S/B | Hillslope | EC₅₀ pM |
|---|---|---|---|
| SEQ ID NO: 77 | 13.8 | 1.874 | 10.0 |
| SEQ ID NO: 87- 1 day | 13.6 | 1.421 | 67.6 |
| SEQ ID NO: 87- 2 day | 14.1 | 2.102 | 30.0 |
| SEQ ID NO: 87- 3 day | 9.8 | 1.825 | 31.3 |
| SEQ ID NO: 87- 5 day | 10.3 | 2.102 | 17.4 |
| SEQ ID NO: 87- 8 day | 8.5 | 2.172 | 14.4 |

### Example 12: Assay of Met(0) and Deamidated Analogs by In Vitro Assay

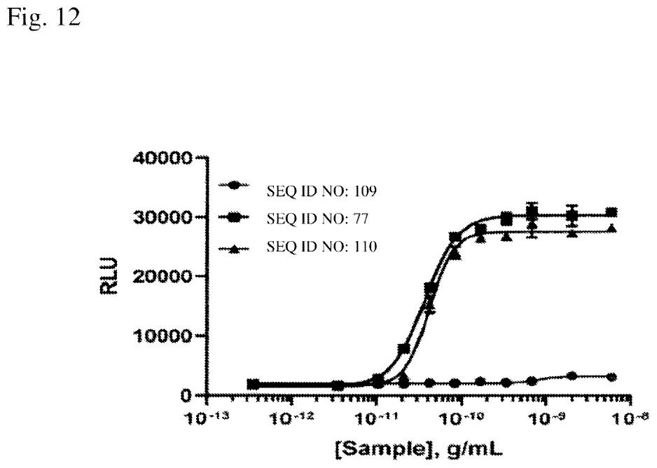

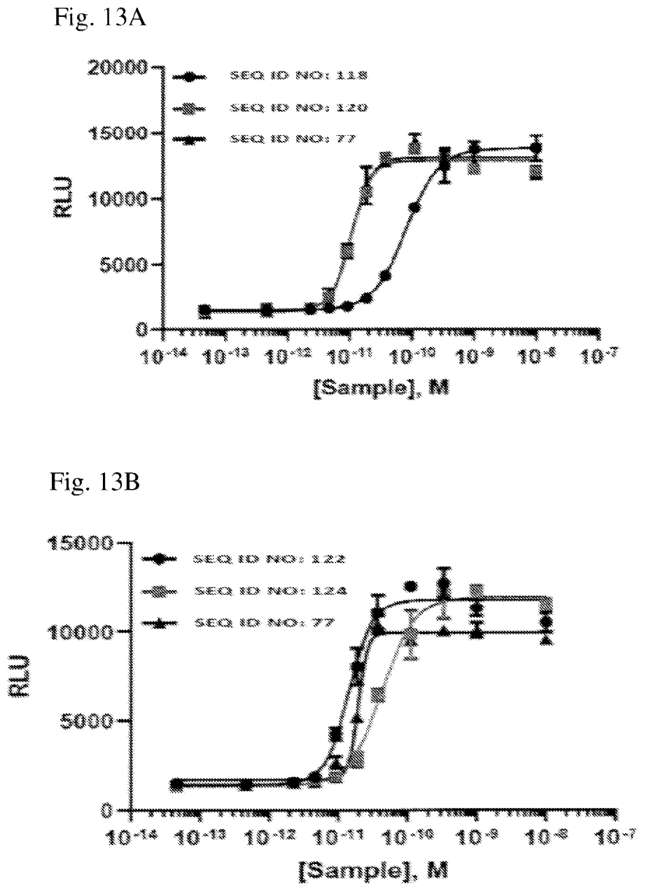

The assessment of the bioactivity of PTH analogs that we site-specifically modified by oxidation to methionine sulfoxide (SEQ ID No 109 and 110) or deaminated from the native Asn or Gln to the respective carboxylic acid Asp or Glu (SEQ ID No 1118, 120, 122 and 124) was conducted in a proprietary commercial assay as contracted with Eurofins-Discover Rx Corporation win a cAMP Hunter® Teriparatide Bioassay (95-0118Y2). An 11-point dose curve in agonist mode was conducted for each individual peptide at each concentration. Every sample was run at each dose in triplicate. The results in Tables 17, 18 and 19, as well as FIGS. 12, 13A and 13B.

TABLE 17
In vitro results associated with FIG. 12

| Sample | S/B | Hillslope | EC₅₀ pM |
|---|---|---|---|
| SEQ ID NO: 77 | 16.9 | 2.237 | 36.6 |
| SEQ ID NO: 109 | 1.7 | — | — |
| SEQ ID NO: 110 | 14.2 | 3.034 | 41.7 |

TABLE 18
In vitro results associated with FIG. 13A

| Sample | S/B | Hillslope | EC₅₀ pM |
|---|---|---|---|
| SEQ ID NO: 77 | 10.4 | 2.687 | 10.7 |
| SEQ ID NO: 118 | 9.4 | 1.648 | 80.3 |
| SEQ ID NO: 120 | 10 | 2.804 | 10.9 |

TABLE 19
In vitro results associated with FIG. 13B

| Sample | S/B | Hillslope | EC₅₀ pM |
|---|---|---|---|
| SEQ ID NO: 77 | 7.2 | 2.687 | 19.3 |
| SEQ ID NO: 122 | 8.2 | 2.522 | 14.3 |
| SEQ ID NO: 124 | 8.9 | 1.797 | 43.1 |

### Example 13: Serum Calcium, Phosphate, Body Weight, Food Intake and Drug Levels in Sprague Dawley Rats Following Repeat Subcutaneous Administration of 20 nmol/kg of SEQ ID NO: 77, 20 or 40 nmol/kg of SEQ ID NO: 87

This experiment measured the effect of repeat daily dosing of SEQ ID NO: 77 AND SEQ ID NO: 87 on serum calcium and phosphate in Sprague-Dawley rats. Female Sprague-Dawley rats were single or double-housed and randomly assigned to four groups (n=5-6 per group) as described in Table 20, and then weighed.

TABLE 20
Treatment Groups and Sampling Times

| | | Dose | Number | PD | PK |
|---|---|---|---|---|---|
| Group | Compound | (nmol/kg) | of Rats | Time Points | Time Points |
| A | SEQ ID NO: 77 | 20 | 6 | 0, 1, 2, 3, 4, 7 | 1, 2, 4, 7 |
| B | Vehicle | — | 5 | 0, 1, 2, 3, 4, 7 | 1, 2, 4, 7 |
| C | SEQ ID NO: 87 | 20 | 5 | 0, 1, 2, 3, 4, 7 | 1, 2, 4, 7 |
| D | SEQ ID NO: 87 | 40 | 6 | 0, 1, 2, 3, 4, 7 | 1, 2, 4, 7 |

Rats were injected subcutaneously once daily for 7 days with either vehicle (sterile 0.9% NaCl solution; n=5), SEQ ID NO: 77 (20 nmol/kg; n=6) or SEQ ID NO: 87 (20 or 40 nmol/kg; n=5 and 6, respectively). After the treatment ceased, the animals were monitored on days 9 and 11 (washout period).

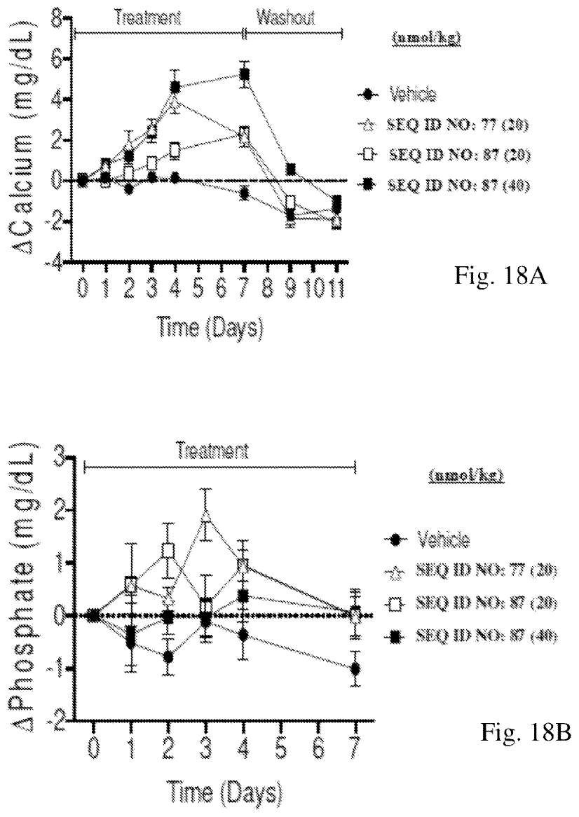

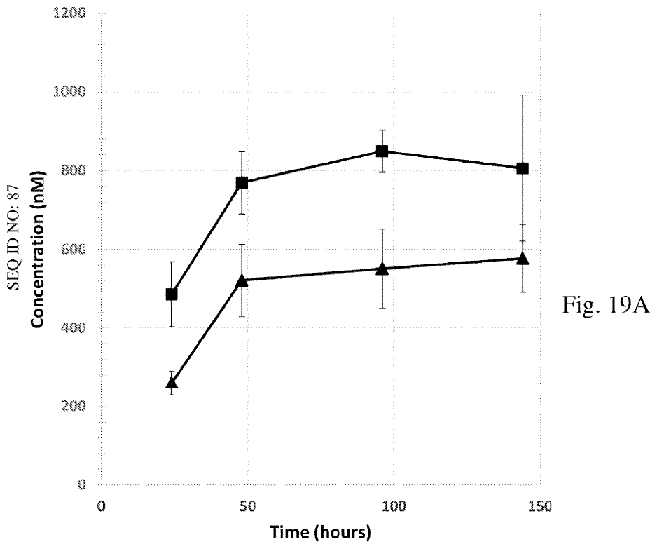

Blood samples were collected via tail laceration from all animals for serum calcium and phosphate analysis prior to vehicle, SEQ ID NO: 77 or SEQ ID NO: 87 administration (0 hour) and after administration (1, 2, 3, 4, 7 days) for determination of total calcium (Ca) and inorganic phosphorous (Pi) in serum using commercially available colorimetric assays. Body weight and food intake were measured on days 0, 1, 3, 5 and 7. Ca was measured on days 0, 1, 2, 3, 4, 7, 9 and 11. Pi was determined on the same samples, except for days 9 and 11. The measurement of serum SEQ ID NO: 87 and SEQ ID NO: 77 was performed by LCMS and the data are shown in FIGS. 18A and 18B.

Serum Ca levels gradually increased in all three treated groups during the treatment period and returned to the initial values at the end of the washout period (FIG. 18A). Changes in serum Pi did not differ significantly except for day 3, where the level of the SEQ ID NO: 77 group differed significantly from those of vehicle and SEQ ID NO: 87 rats treated at 40 nmol/kg (FIG. 18B).

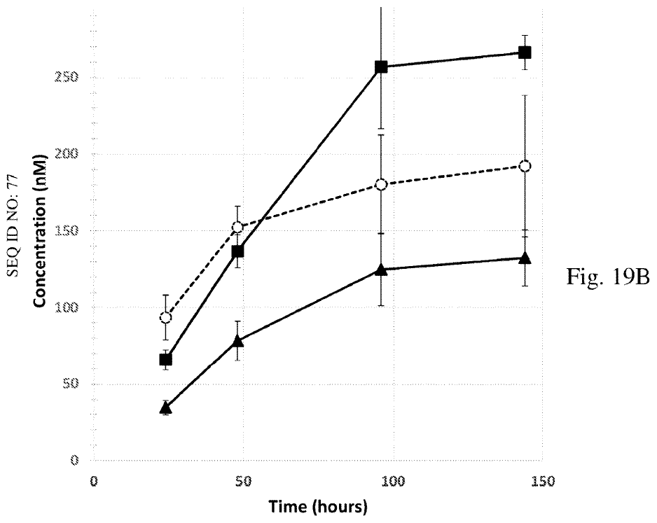

The plasma levels of SEQ ID NO: 77 and SEQ ID NO: 87 demonstrated a dose proportional increase following dosing (FIGS. 19A and 19B). Each peptide reached steady state levels after four daily administrations. The relative concentration of SEQ ID NO: 77 was approximately one-fourth the level of SEQ ID NO: 87. The relative concentration of SEQ ID NO: 77 at steady state when derived from administration of the prodrug SEQ ID NO: 87 was approximately 70% of that achieved by direct administration of SEQ ID NO: 77.

### Example 14: Measuring Serum Calcium, Body Weight and Food Intake in Sprague Dawley Rats Following Subcutaneous Administration of 4, 8 or 12 nmol/kg of SEQ ID NO: 87

This experiment measured the effect of repeat daily dosing with SEQ ID NO: 87 at 4, 8 and 12 nmol/kg for 28 days in female Sprague-Dawley rats. Changes in body weight and food intake were also measured.

Rats were randomly distributed among four groups (n=10) and injected subcutaneously once daily for 21 days with either vehicle or SEQ ID NO: 87 (4, 8, 12 nmol/kg). Body weight and food intake were measured on Day 0 (immediately prior to peptide administration), 1, 3, 7, 10, 14, 17, 21, 24, 28, 31, and 35. Serum Ca was measured at the start of the experiment and on Days 7, 14, 21, 28, 29, 30, 31, 32 and 34. Blood samples were collected via tail laceration for determination of total Ca in serum using commercially available colorimetric assays. Serum samples were collected for pharmacokinetic measurement of SEQ ID NO: 87 and the resultant drug SEQ ID NO: 77.

All rats in the study, including the vehicle-treated animals, experienced a slight increase in total body weight that was no more than 2% based upon a starting body weight of 272.6 gm. Cumulative food intake was dose proportionally increased by a magnitude consistent with the relative increase in body weight when compared to the vehicle-treated rats.

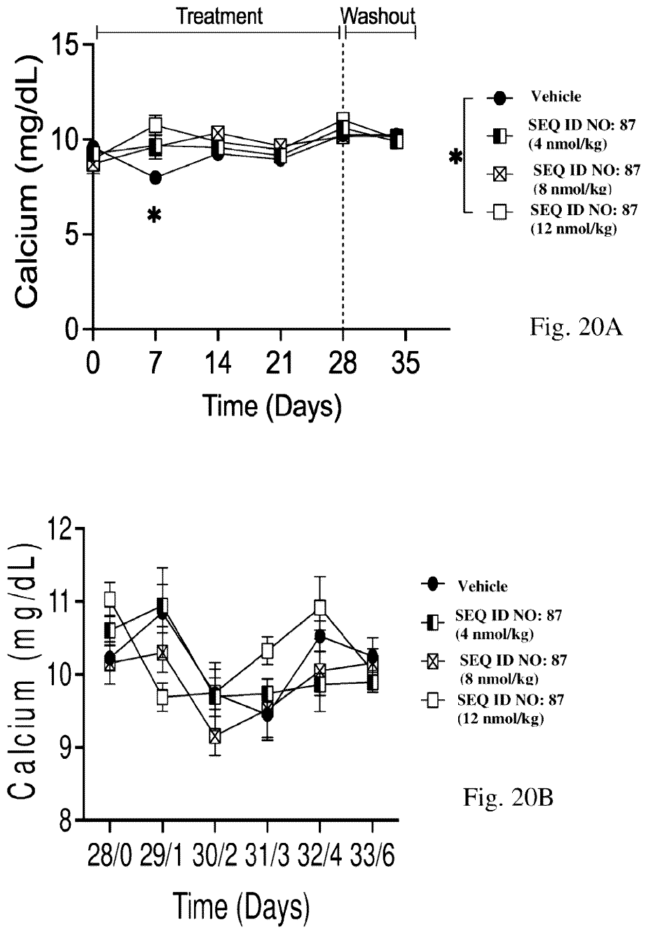

In all treated rats, including the vehicle control, there was an increase in serum Ca over the course of the study (FIG. 20A). The absolute increase was 0.636, 1.356, 1.452 and 2.072 mg/dL for the vehicle, 4, 8, and 12 nmol/kg treated rats, respectively. All serum Ca levels remained within the normal range and, as a percentage increase, the vehicle-treated rats were 6.6%. Increases of 14.7%, 16.7% and 23% were recorded for the increasing doses of SEQ ID NO: 87 (4, 8, 12 nmol/kg, respectively). The relative increase in Ca in this dose range was less than proportional to the increase in dose, as a threefold increase in dose from 4 to 12 nmol/kg demonstrated less than a twofold relative increase in serum Ca (a 56% increase). On Day 34, the serum Ca levels in all the SEQ ID NO: 87 rats had diminished in concentration (FIG. 20B). The vehicle-treated animals were unchanged, with Ca levels increased 6.9% relative to study initiation. The low dose-treated rats showed an increase in serum Ca of 6.9% relative to study start. The mid- and high-dose rats displayed increased serum Ca of 11.7% and 12.2%, respectively. All animals appeared healthy.

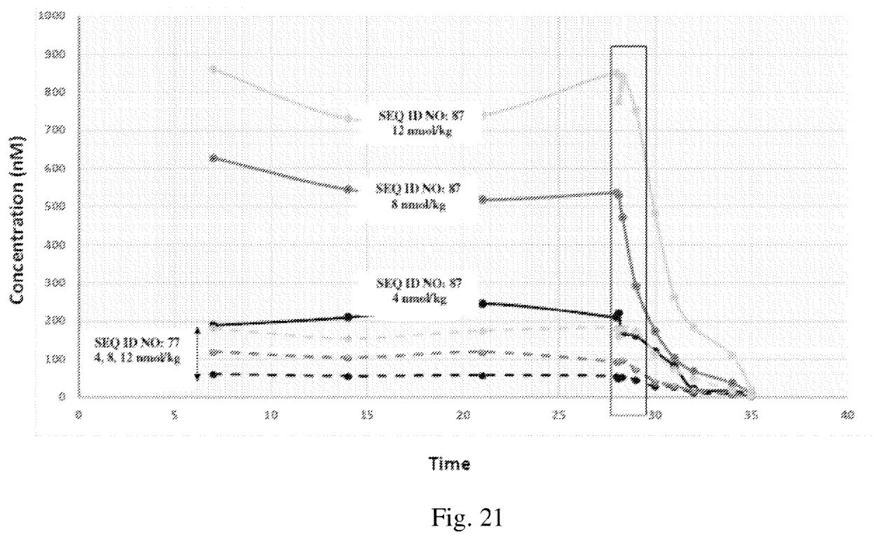

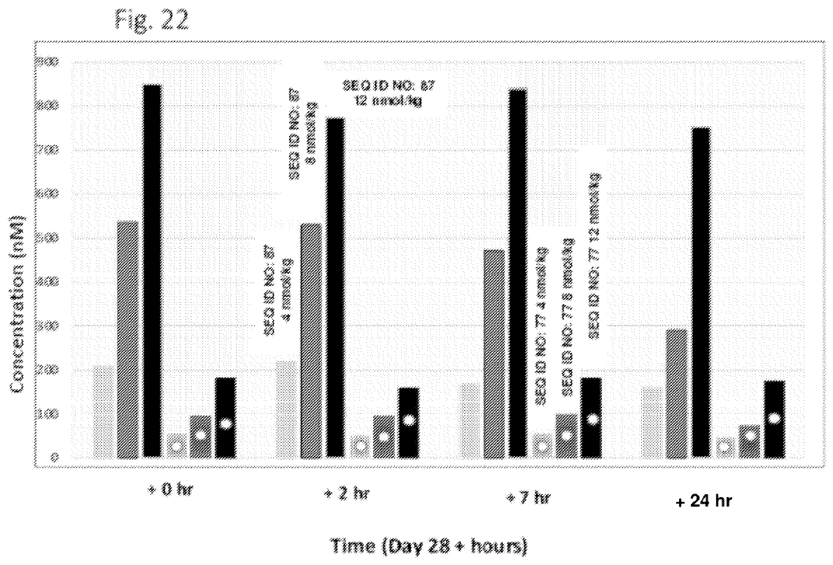

Weekly determination of SEQ ID NO: 87, and subsequent SEQ ID NO: 77, plasma concentration began on Day 7 after steady state levels had been achieved by daily dosing. There was a dose proportional increase in both peptides with the pro-drug (SEQ ID NO: 87) at any of the three administered doses being fourfold higher than the resultant drug (SEQ ID NO: 77). The absolute and relative concentration of the two peptides across the three doses was maintained in the period of 7-28 days with repeat daily dosing. Over the course of the subsequent seven days, the disappearance of these two peptides was monitored by LCMS. In the first twenty-four-hour period following the last dose, the plasma concentration of each peptide was measured at 2, 7 and 24 hours post dosing. The results demonstrated that once steady concentrations had been achieved there was little change in daily drug exposure with each subsequent dose. These results are shown in FIGS. 21 and 22.

### Example 15: Measuring Serum Calcium Levels in Parathyroidectomized Sprague-Dawley Rats after Repeat Subcutaneous Administration of 15, 25, or 40 nmol/kg of SEQ ID NO: 87

This experiment measured the profile of serum calcium following repeat subcutaneous injection of the parathyroid hormone analogue prodrug, SEQ ID NO: 87 on serum and urine calcium in female Sprague-Dawley rats that had their parathyroid gland surgically removed. Rats were randomly distributed among 5 groups and injected subcutaneously for four consecutive days with either vehicle (sterile 0.9% NaCl solution; n=6), or SEQ ID NO; 87 (10, 25, 40 nmol/kg; n=8). Two separate vehicle treated groups of rats were studied. The first represented control rats surgically managed like the remaining rats, but their parathyroid gland was not removed (sham control). The second vehicle control group represented rats that were surgically managed in the same manner as the SEQ ID NO: 87 treated rats. Following surgery and one week prior to testing all the rats were placed on a defined diet at a specific calcium concentration.

Blood samples were collected prior to vehicle or SEQ ID NO: 87 administration (0 hour) and after administration (24, 48, 72, 96, 144 hours) via tail laceration for determination of total calcium (Ca) in serum using commercially available colorimetric assays following the manufacturer's recommendations (Stanbio Laboratory: Calcium LiquiColor #0155). A 96-well plate spectrophotometer (SpectraMax M5, Molecular Devices) was employed for detection. The Calcium assay contained a color reagent (Cat. No. 0156) and a Calcium Standard (10 mg/dL; Cat. No. 0157). Absorbance (Ab) was measured at 650 nm wavelength. Manufacturer provided linearity up to 15 mg/dL. The serum calcium was measured immediately prior to injection (time 0 hour), followed by 24, 48, 72, and 96 hours. Results (mg/dL) were calculated as follows: Ab(unknown)/Ab(standard)×10.

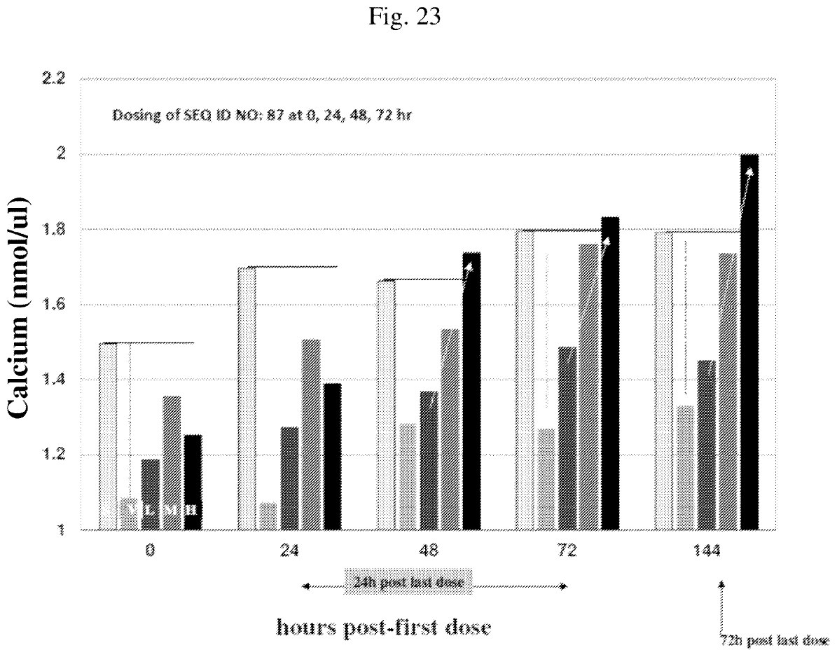

The sham control rats demonstrated a starting serum calcium that was notably increased relative to the vehicle-treated PTx-rats. The starting calcium level was 1.495 nmol/ul as compared to 1.085 nmol/ul in the vehicle control group of the PTx rats. This represented a relatively higher concentration of 37.8%. The difference between the two control groups was largely maintained through the course of the experiment, although there was a nearly 0.3 nmol/ul increase in both control groups. The SEQ ID NO: 87 treated rats began treatment at serum calcium concentrations that were intermediate to the two vehicle control groups, being 1.185, 1.354, and 1.251 nmol/ul for treatment groups 10, 25, 40 nmol/kg. This represented 9.2%, 24.8% and 15.3% higher levels respectively than the comparable PTx-rats that constituted the vehicle treated group. After 48 hours and immediately prior to the third dose, there was a clear dose response observed in the PTx-treated rats with the highest dose of 40 nmol/kg slightly exceeding that of the sham control rats. This relationship was maintained at 72 hours where the intermediate dose of 25 nmol/kg was insignificantly different from the sham control rats. At 144 hours, which was 72 hours following the last dose, the PTx-rats continued to exhibit a dose proportional difference in serum calcium. The difference between the starting level of the PTx-vehicle treated rats and mid-dose groups doubled through this period of therapy, and for the highest dose group, it more than tripled to a point where it exceeded the sham control by more than 0.2 nmol/ml and results are shown in FIG. 23.

### Example 16: Measuring Serum and Urine Calcium Levels in Parathyroidectomized Sprague-Dawley Rats after Repeat Subcutaneous Administration of 15, 25, or 40 nmol/kg of SEQ ID NO: 87

This experiment measured the profile of serum calcium following repeat subcutaneous injection of the parathyroid hormone analogue prodrug, SEQ ID NO: 87 in female Sprague-Dawley rats that had their parathyroid gland surgically removed. Rats were randomly distributed among 5 groups and injected subcutaneously for ten consecutive days with either vehicle or SEQ ID NO; 87 (10, 25, 40 nmol/kg; n=8). Two separate vehicle treated groups of rats were studied. The first represented control rats surgically managed like the remaining rats, but their parathyroid gland was not removed (sham control). The second vehicle control group represented rats that were surgically managed in the same manner as the SEQ ID NO: 87 treated rats. Following surgery and one week prior to testing all the rats were placed on a defined diet at a specific calcium concentration.

Blood samples were collected via tail laceration for determination of total calcium (Ca) in serum using commercially available colorimetric assays following the manufacturer's recommendations (Stanbio Laboratory: Calcium LiquiColor #0155). A 96-well plate spectrophotometer (SpectraMax M5, Molecular Devices) was employed for detection. The Calcium assay contained a color reagent (Cat. No. 0156) and a Calcium Standard (10 mg/dL; Cat. No. 0157). Absorbance (Ab) was measured at 650 nm wavelength. Manufacturer provided linearity up to 15 mg/dL. The serum calcium was measured immediately prior to injection (time 0 hour), followed by 24, 48, 72, and 96 hours. Results (mg/dL) were calculated as follows: Ab(unknown)/Ab(standard)×10.

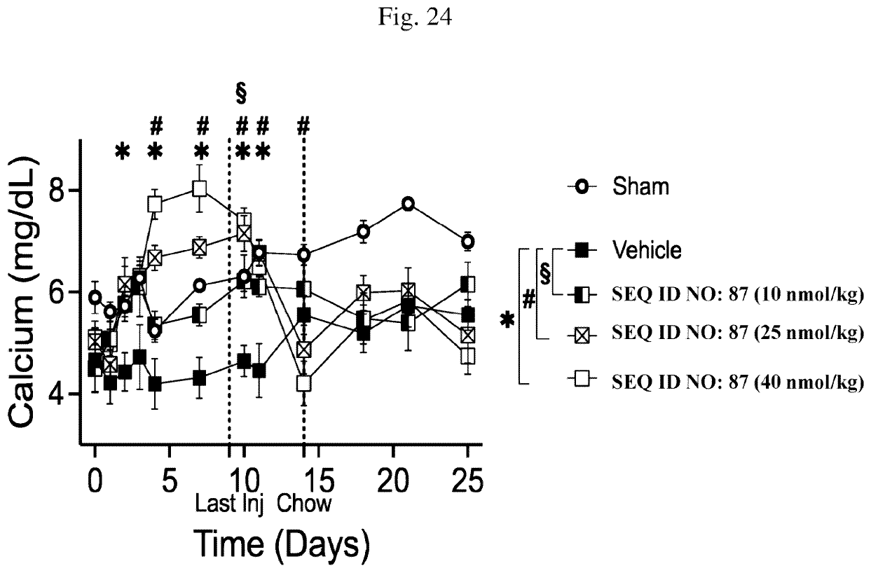

The sham control rats demonstrated a starting serum calcium that was notably increased relative to the vehicle-treated PTx-rats. The starting calcium level was 5.896 mg/dl, as compared to 4.66 mg/Dl in the vehicle control group of the PTx rats. This represented a relatively higher concentration of 26.5%. The difference between these two control groups was largely maintained through the course of the experiment. The SEQ ID NO: 87 treated rats began treatment at serum calcium concentrations that were intermediate to the two vehicle control groups, being 4.5, 5.028 and 5.108 mg/dl for treatment groups 10, 25, 40 nmol/kg (FIG. 24). This represented, respectively, a decreased level of 3.4% in the lowest dose group, and an increased level of 7.9% and 9.6% for the mid and higher dose groups. After two days, and immediately prior to the third dose, all the SEQ ID NO: 87 treated rats displayed serum calcium levels virtually identical to sham control rats. At four and seven days, the mid and high-dose PTx-rats displayed serum calcium levels that matched or exceeded what was observed in the sham control rats. There was a dose proportional difference in the rats treated with SEQ ID NO: 87, with the highest dose rats being greatest in magnitude and the lowest dose rats being lowest in serum calcium. The PTx-treated rats maintained a heightened serum calcium that paralleled the sham treated rats through Days 10 and 11. At Day 14, and beyond through Day 21, the PTx-rats returned to serum calcium levels that matched each other, including the vehicle treated rats. These calcium values were equally reduced in serum calcium on Day 21, relative to the sham rats, as the difference that existed on Day 0 at start of the study.

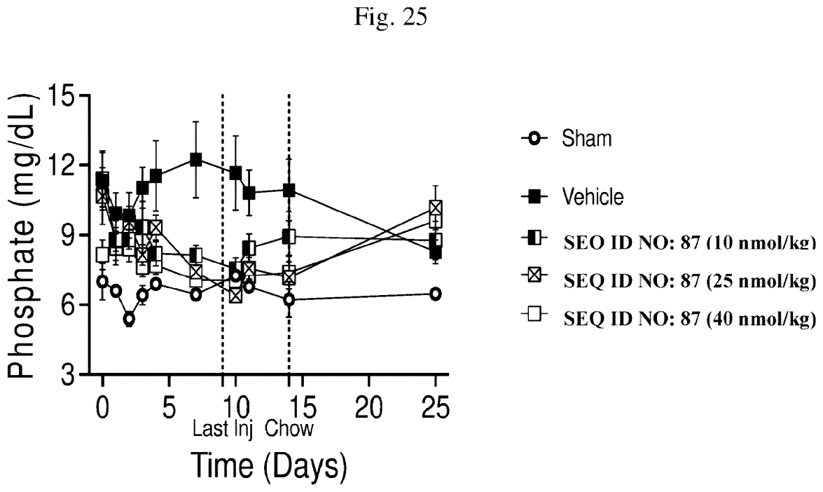

Determination of phosphate levels was conducted with sampling at near-identical times to the serum calcium, differing only in the last days of the washout period. The response to SEQ ID NO: 87 paralleled that previously described for serum calcium but with phosphate decreasing at each dose relative to the PTx-rats treated with vehicle (FIG. 25). With increased time of treatment, the phosphate levels in the SEQ ID NO: 87 treated rats matched that of the sham control rats. The sham control rats demonstrated a starting serum phosphate that was notably decreased relative to the vehicle-treated PTx-rats. The starting phosphate level was 7.003 mg/dl, as compared to 11.317 mg/dl in the vehicle control group of the PTx rats. This represented a relatively lower concentration of 38.1%. The difference between the two control groups was maintained through the course of the experiment. The SEQ ID NO: 87 treated rats began treatment at serum phosphate concentrations that were intermediate to the two vehicle control groups, being 11.412, 10.673 and 8.152 mg/dl for treatment groups 10, 25, 40 nmol/kg, respectively. This represented, respectively, a 1.0% increase at low-dose and a 5.7% and 28% decreased level at mid and high-dose. After seven days, all SEQ ID NO: 87 PTx-treated rats exhibited serum phosphate levels comparable to sham control rats. Efficacy was fully maintained through observation at Day 10, one day after the last administered dose. After two weeks, the mid and high dose rats exhibited comparably decreased phosphate levels while the lowest dose rats were increased relative to their nadir at ten days. Twenty-five days after study initiation, all PTx-rats aligned as a group with increased phosphate, as compared to sham control rats.

### Example 17: Comparative Pharmacokinetics in Cynomolgus Monkeys Following a Single Subcutaneous Dose of SEQ ID 87

The pharmacokinetic profile of PTH analogue SEQ ID No 87 was studied following single subcutaneous dose administration to cynomolgus monkeys by methodology detailed in Example 8. A total of three monkeys were used at each test dose. The monkeys ranged in age from 2-4 years old at beginning of the study and having a weight of at least 2.5 kg. The dose volume was 0.3 mL/kg. Individual dose volumes were calculated based on the animals' most recently recorded body weight.

At least a 0.5 mL sample of blood was collected from all animals at predose, and at 6, 24, 48, 72, 96, 120-, 144-, 168-, 192-, 216-240-, 264, 336-hours following dosing. Animals were not fasted prior to collection. Blood was be collected via the femoral vein (or another appropriate site). Blood samples were maintained at 5±3° C. (wet ice or equivalent) and centrifuged at 5±3° C. within 1 hour of the collection of each blood sample. The resultant plasma was transferred to a tube and then stored under conditions set to maintain −75±15° C. until analysis.

### Pharmacokinetic Evaluation

Pharmacokinetic mean concentration-time data was analyzed using non-compartmental methods (Phoenix® WinNonlin® Version 7.0 or higher) based on the route of administration. The following parameters were calculated whenever possible and as data allows: Cmax, Tmax, and AUC. Descriptive statistics were generated using Phoenix WinNonlin.

### Results

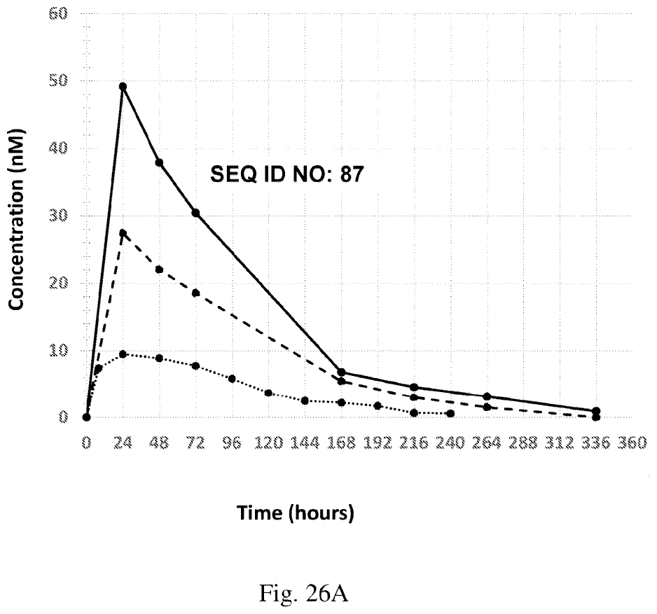

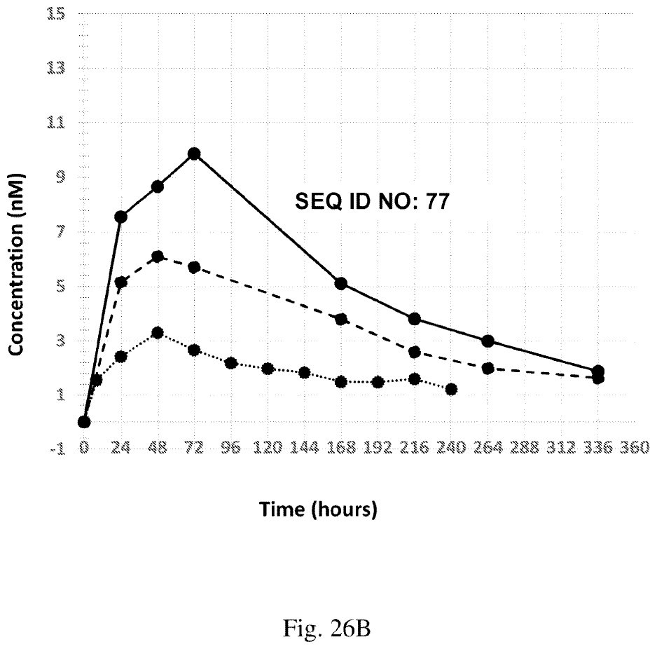

Results from these pharmacokinetic (PK) studies are provided in FIGS. 26A and 26B wherein the concentration of the administered prodrug form and its active drug form (produced after in vivo non-enzymatic cleavage of the dipeptide prodrug element) were measured over time. Monkeys were administered a single subcutaneous dose of prodrug PTH-analog SEQ ID No 87 at 2.5, 3.75 and 5 nmol/kg and the concentration of the prodrug (SEQ ID No 87, FIG. 26A) along with the corresponding cleavage product, SEQ ID NO: 77 (“the drug”) were measured over the next 336 hours after administration. There was a dose proportional increase in the concentration of SEQ ID NO: 87 that peaked at 24 hours and did not return to starting concentrations until after one week (FIG. 26A). The peptide SEQ ID NO: 77 that results from conversion from SEQ ID NO: 87 peaks at 48-72 hours and its concentration remains at approximately ⅔ the Cmax concentration when assessed at one week. The concentration of SEQ ID NO: 77 remained elevated relative to the starting concentration even at two-weeks post-dosing (FIG. 26B).

1. A conjugate comprising a PTH peptide, and a non-enzymatic self-cleaving dipeptide covalently linked to the N-terminal alpha amine of said PTH peptide via an amide bond, wherein said PTH peptide comprises the amino acid sequence of
(SEQ ID NO: 16)
SVSEIQLMHNLGKHLNSMERVEWLRKKLQDVHX₃₃,
wherein
X₃₃ is an amino acid comprising a side chain comprising -{gamma glutamic acid-[COCH₂(OCH₂CH₂)ₖ—NH]_{q}-gamma glutamic acid}-CO(CH₂)₁₄₋₂₀COOH; and
said self-cleaving dipeptide comprises the general structure: 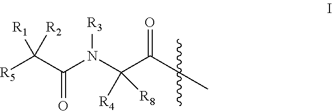
wherein
R₁ comprises —NH—CO(CH₂)₁₄₋₂₀COOH, —NH-[spacer]-CO(CH₂)₁₄₋₂₀COOH, —NH—CO(CH₂)₁₄₋₂₀CH₃, or —NH-[spacer]-CO(CH₂)₁₄₋₂₀CH₃;
R₂, R₄ and R₈ are each H;
R₃ is CH₃;
R₅ is NH₂;
q is 2 or 4 and
k is 2.

2. The conjugate of claim 1, wherein
X₃₃ is an amino acid comprising a side chain of -{gamma glutamic acid-[COCH₂(OCH₂CH₂)₂NH—COCH₂(OCH₂CH₂)₂]NH-gamma glutamic acid}-CO(CH₂)₁₆COOH; and
R₁, comprises NH-{gamma glutamic acid-[COCH₂(OCH₂CH₂)₂NH—COCH₂(OCH₂CH₂)₂]NH-gamma glutamic acid}-CO(CH₂)₁₆COOH.

3. A pharmaceutical composition comprising the conjugate of claim 1, or pharmaceutically acceptable salt thereof, and a pharmaceutically acceptable carrier.

4. A conjugate comprising a PTH peptide and a non-enzymatic self-cleaving dipeptide covalently bound to said PTH peptide via an amide bond,
said PTH peptide comprising an amino acid sequence selected from the group consisting of
(SEQ ID NO: 16)
SVSEIQLMHNLGKHLNSMERVEWLRKKLQDVHX₃₃,
wherein said non-enzymatic self-cleaving dipeptide comprising the general structure A-B—;
wherein
A is an amino acid or an acylated amino acid;
B is an N-alkylated amino acid;
wherein said acylated amino acid of each of X₃₃ and A is independently selected from an amino acid comprising a C16-C30 fatty acid or C16-C30 diacid covalently linked to the amino acid side chain, optionally via a spacer, and said non-enzymatic self-cleaving dipeptide is linked to said PTH peptide through formation of an amide bond between B and the N-terminal alpha amine of said PTH peptide, further wherein said optional spacer of each of X₃₃ and A comprises one or more linker moieties independently selected from the group consisting of a gamma glutamic acid, and COCH₂(OCH₂CH₂)ₖNH, wherein k is an integer selected from the range of 1-8;
with the proviso that when A is a non-acylated amino acid, then A is an amino acid in the D-stereochemical configuration.

5. The conjugate of claim 4, wherein X₃₃ is an amino acid comprising a C16-C22 fatty acid or C16-C22 diacid covalently linked to the side chain of the amino acid, optionally via a spacer.

6. The conjugate of claim 4, wherein A is selected from the group consisting of Lys, dLys, acylated-Lys and acylated-dLys wherein said self-cleaving dipeptide is covalently linked to the N-terminal alpha amine of the PTH peptide.

7. The conjugate of claim 4, wherein said acylated amino acid of each of X₃₃ and A comprises a C16-C30 fatty acid or C16-C30 diacid covalently linked to the amino acid side chain via a spacer, wherein the spacer of each of X₃₃ and A is independently selected from a gamma glutamic acid-gamma glutamic acid dipeptide, (Xaa)-[COCH₂(OCH₂CH₂)ₖNH]_{q}-gamma glutamic acid and a gamma glutamic acid-[COCH₂(OCH₂CH₂)ₖNH]_{q}-gamma glutamic acid, wherein
Xaa is selected from Arg, Tyr(OPO₃H₂), and hCys(SO₃H);
k is an integer selected from the range of 1-8; and
q is an integer selected from the range of 1-8, optionally wherein k is 2 and q is selected from the range of 1-4.

8. The conjugate of claim 4, wherein the acylated amino acid of A and X₃₃ are independently selected from cysteine, homocysteine, ornithine lysine and d-lysine wherein the side chain of said cysteine, homocysteine, ornithine lysine or d-lysine is covalently linked to a C16-C22 fatty acid or C16-C22 diacid, optionally through a spacer, wherein the optional spacer of each of A and X₃₃ comprises a gamma glutamic acid linkage.

9. The conjugate of claim 4, wherein the acylated amino acid of A and X₃₃ are each independently selected from lysine or d-lysine wherein the side chain of said lysine or d-lysine is covalently linked to a C16-C22 fatty acid or C16-C22 diacid, optionally through a spacer comprising a gamma glutamic acid linkage.

10. The conjugate of claim 9, wherein the acylated amino acid of A is d-lysine and X₃₃ is lysine.

11. The conjugate of claim 9, wherein said spacer of A and X₃₃ are each independently selected from compounds comprising the structure: gamma glutamic acid-[COCH₂(OCH₂CH₂)ₖNH]_{q}-gamma glutamic acid, wherein k is an integer selected from 2, 4 or 8 and q is an integer selected from 1, 2, 4 or 8.

12. The conjugate of claim 11, wherein k is 2 and q is 2 or 4.

13. The conjugate of claim 4, wherein A-B comprises the structure: 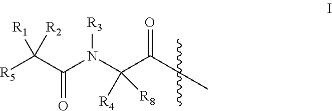
wherein
R₁ comprises a side chain comprising a C16-C30 fatty acid or C16-C30 diacid is covalently linked to said side chain, optionally via a spacer selected from the group consisting of a gamma glutamic acid, a gamma glutamic acid-gamma glutamic acid dipeptide, and a gamma glutamic acid-[COCH₂(OCH₂CH₂)ₖNH]_{q}-gamma glutamic acid, wherein
k is an integer selected from the range of 1-8; and
q is an integer selected from the range of 1-8, optionally wherein k is 2 and q is selected from the range of 1-8;
R₂, R₄ and R₈ are independently H, or C₁-C₄ alkyl;
R₃ is C₁-C₆ alkyl; and
R₅ is NH₂.

14. The conjugate of claim 13, wherein the chemical cleavage half-life (t_{1/2}) of A-B from said PTH peptide is at least about 48 to 168 hours in standard PBS solution under physiological conditions.

15. The conjugate of claim 13, wherein
R₁ comprises a side chain selected from the group consisting of C₁-C₈ alkyl, (C₁-C₄ alkyl)OH, (C₁-C₄ alkyl)SH, (C₁-C₄ alkyl)COOH, and (C₁-C₄ alkyl)NH₂;
R₂ and R₈ are each H;
R₄ is H, or CH₃;
R₃ is CH₃ and
R₅ is NH₂.

16. The conjugate of claim 13, wherein
R₁ comprises -[spacer]-CO(CH₂)₁₄₋₂₀COOH, -[spacer]-CO(CH₂)₁₄₋₂₀CH₃, —CO(CH₂)₁₄₋₂₀COOH, or —CO(CH₂)₁₄₋₂₀CH₃;
R₂ and R₈ are each H;
R₄ is H, or CH₃;
R₃ is CH₃ and
R₅ is NH₂, wherein said [spacer] is a linking moiety selected from the group consisting of a gamma glutamic acid, a gamma glutamic acid-gamma glutamic acid dipeptide, and a gamma glutamic acid-[COCH₂(OCH₂CH₂)ₖNH]_{q}-gamma glutamic acid, wherein k is an integer selected from the range of 2-4 and q is an integer selected from the range of 1-8.

17. The conjugate of claim 16, wherein R₁ is (C₁-C₄ alkyl)NH-[spacer]-CO(CH₂)₁₄₋₂₀COOH, wherein said [spacer] is a linking moiety comprising the structure gamma glutamic acid-[COCH₂(OCH₂CH₂)ₖNH]_{q}-gamma glutamic acid, wherein k is 2 or 4 and q is 1, 2 or 4.

18. The conjugate of claim 16, wherein R₁ is (C₄ alkyl)NH-{gamma glutamic acid-[COCH₂(OCH₂CH₂)₂NH—COCH₂(OCH₂CH₂)₂]NH-gamma glutamic acid}-CO(CH₂)₁₄₋₂₀COOH.

19. The conjugate of claim 4, wherein the first amino acid of the cleavable dipeptide is an amino acid in the D-stereochemical configuration.

20. A pharmaceutical composition comprising the conjugate of claim 4, or pharmaceutically acceptable salt thereof, and a pharmaceutically acceptable carrier.


SEQUENCE LISTING (149 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 33 aa, Homo sapiens
  VARIANT 33: note = MISC_FEATURE - Xaa at position 33 is any acylated amino acid
SVSEIQLMHNLGKHLNSMERVEWLRKKLQDVHX

SEQ ID NO: 3 - protein, 34 aa, Homo sapiens
  VARIANT 34: note = MISC_FEATURE - Xaa at position 34 is any acylated amino acid
SVSEIQLMHNLGKHLNSMERVEWLRKKLQDVHNX

SEQ ID NO: 4 - protein, 32 aa, Homo sapiens
  VARIANT 12: note = MISC_FEATURE - Xaa at position 12 is isobutyric acid (Aib) or Gly
  VARIANT 13: note = MISC_FEATURE - Xaa at position 13 is Arg, Glu, Asp or Lys
  VARIANT 16: note = MISC_FEATURE - Xaa at position 16 is isobutyric acid (Aib) or Asn
  VARIANT 17: note = MISC_FEATURE - Xaa at position 17 is amino isobutyric acid (Aib) or Ser
  VARIANT 26: note = MISC_FEATURE - Xaa at position 26 is Arg, Glu, Asp or Lys
  VARIANT 27: note = MISC_FEATURE - Xaa at position 27 is Arg, Glu, Asp or Lys
  VARIANT 31: note = MISC_FEATURE - Xaa at position 31 is Gly or Val
SVSEIQLMHNLXXHLXXMERVEWLRXXLQDXH

SEQ ID NO: 5 - protein, 32 aa, Homo sapiens
  VARIANT 13: note = MISC_FEATURE - Xaa at position 13 is Arg, Glu, Asp or Lys
  VARIANT 26: note = MISC_FEATURE - Xaa at position 26 is Arg, Glu, Asp or Lys
  VARIANT 27: note = MISC_FEATURE - Xaa at position 27 is Arg, Glu, Asp or Lys
  VARIANT 31: note = MISC_FEATURE - Xaa at position 31 is Gly or Val
SVSEIQLMHNLGXHLNSMERVEWLRXXLQDXH

SEQ ID NO: 6 - protein, 32 aa, Homo sapiens
  VARIANT 12: note = MISC_FEATURE - Xaa at position 12 is isobutyric acid (Aib) or Gly
  VARIANT 16: note = MISC_FEATURE - Xaa at position 16 is isobutyric acid (Aib) or Asn
  VARIANT 17: note = MISC_FEATURE - Xaa at position 17 is amino isobutyric acid (Aib) or Ser
SVSEIQLMHNLXKHLXXMERVEWLRKKLQDVH

SEQ ID NO: 7 - protein, 32 aa, Homo sapiens
SVSEIQLMHNLGKHLNSMERVEWLRKKLQDVH

SEQ ID NO: 8 - protein, 34 aa, Homo sapiens
  VARIANT 33: note = MISC_FEATURE - Xaa at position 33 is any acylated amino acid
SVSEIQLMHNLGKHLNSMERVEWLRKKLQDVHXF

SEQ ID NO: 9 - protein, 34 aa, Homo sapiens
SVSEIQLMHNLGKHLNSMERVEWLRKKLQDVHNF

SEQ ID NO: 10 - protein, 36 aa, Homo sapiens
  SITE 1: note = MISC_FEATURE - Xaa at position 1 is acylated dLys
XGSVSEIQLMHNLGKHLNSMERVEWLRKKLQDVHNF

SEQ ID NO: 11 - protein, 36 aa, Homo sapiens
  SITE 1: note = MISC_FEATURE - Xaa at position 1 is acylated dLys
  SITE 2: note = MISC_FEATURE - Xaa at position 2 is N-methyl Gly
XXSVSEIQLMHNLGKHLNSMERVEWLRKKLQDVHNF

SEQ ID NO: 12 - protein, 35 aa, Homo sapiens
  VARIANT 35: note = MISC_FEATURE - Xaa at position 35 is an acylated Lys
  SITE 35: note = MISC_FEATURE - C-terminal carboxyl group replaced with an amide
SVSEIQLMHNLGKHLNSMERVEWLRKKLQDVHNFX

SEQ ID NO: 13 - protein, 37 aa, Homo sapiens
  SITE 1: note = MISC_FEATURE - Xaa at position 1 is acylated dLys
  SITE 2: note = MISC_FEATURE - Xaa at position 2 is N-methyl Gly
  SITE 37: note = MISC_FEATURE - Xaa at position 37 is an acylated Lys
  SITE 37: note = MISC_FEATURE - C-terminal carboxyl group replaced with an amide
XXSVSEIQLMHNLGKHLNSMERVEWLRKKLQDVHNFX

SEQ ID NO: 14 - protein, 36 aa, Homo sapiens
  SITE 1: note = MISC_FEATURE - Xaa at position 1 is acylated dLys
  SITE 2: note = MISC_FEATURE - Xaa at position 2 is N-methyl Gly
XXSVSEIQLMHNLGKHLNSMERVEWLRKKLQDVHQF

SEQ ID NO: 15 - protein, 35 aa, Homo sapiens; identical to SEQ ID NO: 12
  VARIANT 35: note = MISC_FEATURE - Xaa at position 35 is any acylated amino acid

SEQ ID NO: 16 - protein, 33 aa, Homo sapiens; identical to SEQ ID NO: 2
  SITE 33: note = MISC_FEATURE - Xaa at position 33 is an acylated Lys

SEQ ID NO: 17 - protein, 35 aa, Homo sapiens
  SITE 1: note = MISC_FEATURE - Xaa at position 1 is acylated dLys
  SITE 2: note = MISC_FEATURE - Xaa at position 2 is N-methyl Gly
  SITE 35: note = MISC_FEATURE - Xaa at position 35 is an acylated Lys
XXSVSEIQLMHNLGKHLNSMERVEWLRKKLQDVHX

SEQ ID NO: 18 - protein, 33 aa, Homo sapiens
  SITE 33: note = MISC_FEATURE - Xaa at position 33 is an acylated Lys
SVSEIQLAHNLGKHLNSMERVEWLRKKLQDVHX

SEQ ID NO: 19 - protein, 35 aa, Homo sapiens
  SITE 1: note = MISC_FEATURE - Xaa at position 1 is dLys
  SITE 35: note = MISC_FEATURE - Xaa at position 35 is an acylated Lys
XGSVSEIQLMHNLGKHLNSMERVEWLRKKLQDVHX

SEQ ID NO: 20 - protein, 85 aa, Homo sapiens
  VARIANT 85: note = MISC_FEATURE - Xaa at position 85 is any acylated amino acid
SVSEIQLMHNLGKHLNSMERVEWLRKKLQDVHNFVALGAPLAPRDAGSQRPRKKEDNVLVESHEKSLGEADKADVNVLTKAKSQX

SEQ ID NO: 21 - protein, 38 aa, Homo sapiens
  VARIANT 38: note = MISC_FEATURE - Xaa at position 38 is any acylated amino acid
SVSEIQLMHNLGKHLNSMERVEWLRKKLQDVHNFVALX

SEQ ID NO: 22 - protein, 32 aa, Homo sapiens
SVSEIQLMHNLGKHLNSMERVEWLRKKLQDGH

SEQ ID NO: 23 - protein, 32 aa, Homo sapiens
SVSEIQLMHNLGEHLNSMERVEWLRKKLQDVH

SEQ ID NO: 24 - protein, 32 aa, Homo sapiens
SVSEIQLMHNLGKHLNSMERVEWLREKLQDVH

SEQ ID NO: 25 - protein, 32 aa, Homo sapiens
SVSEIQLMHNLGKHLNSMERVEWLRKELQDVH

SEQ ID NO: 26 - protein, 35 aa, Homo sapiens; identical to SEQ ID NO: 12
  SITE 35: note = MISC_FEATURE - Xaa at position 35 is any acylated amino acid

SEQ ID NO: 27 - protein, 35 aa, Homo sapiens; identical to SEQ ID NO: 19
  SITE 1: note = MISC_FEATURE - Xaa at position 1 is an acylated dLys
  SITE 35: note = MISC_FEATURE - Xaa at position 35 is an acylated Lys

SEQ ID NO: 28 - protein, 6 aa, synthetic construct
  REGION 1..6: note = model hexpeptide for prodrug studies
  VARIANT 1: note = MISC_FEATURE - Xaa at position 1 is His, d-His or N-methyl-His
XSRGTF

SEQ ID NO: 29 - protein, 8 aa, synthetic construct
  REGION 1..8: note = model hexpeptide for prodrug studies
  SITE 2: note = MISC_FEATURE - Xaa at position 2 is Sarcosine
KXHSRGTF

SEQ ID NO: 30 - protein, 35 aa, Homo sapiens; identical to SEQ ID NO: 12
  VARIANT 35: note = MISC_FEATURE - Xaa at position 35 is any acylated amino acid

SEQ ID NO: 31 - protein, 33 aa, Homo sapiens
SVSEIQLMHNLGKHLNSMERVEWLRKKLQDVHN

SEQ ID NO: 32 - protein, 34 aa, Homo sapiens; identical to SEQ ID NO: 9

SEQ ID NO: 33 - protein, 35 aa, Homo sapiens
SVSEIQLMHNLGKHLNSMERVEWLRKKLQDVHNFV

SEQ ID NO: 34 - protein, 36 aa, Homo sapiens
SVSEIQLMHNLGKHLNSMERVEWLRKKLQDVHNFVA

SEQ ID NO: 35 - protein, 37 aa, Homo sapiens
SVSEIQLMHNLGKHLNSMERVEWLRKKLQDVHNFVAL

SEQ ID NO: 36 - protein, 38 aa, Homo sapiens
SVSEIQLMHNLGKHLNSMERVEWLRKKLQDVHNFVALG

SEQ ID NO: 37 - protein, 39 aa, Homo sapiens
SVSEIQLMHNLGKHLNSMERVEWLRKKLQDVHNFVALGA

SEQ ID NO: 38 - protein, 34 aa, Homo sapiens; identical to SEQ ID NO: 3
  SITE 34: note = MISC_FEATURE - Xaa at position 34 is K(gamma-glu-COC16H32CO2H)F-OH

SEQ ID NO: 39 - protein, 147 aa, Homo sapiens
SRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 40 - protein, 35 aa, Homo sapiens; identical to SEQ ID NO: 12
  SITE 35: note = MISC_FEATURE - Xaa at position 35 is dAla that has an amide substituted for the native carboxyl group

SEQ ID NO: 41 - protein, 33 aa, Homo sapiens
VSEIQLMHNLGKHLNSMERVEWLRKKLQDVHNF

SEQ ID NO: 42 - protein, 34 aa, Homo sapiens
GVSEIQLMHNLGKHLNSMERVEWLRKKLQDVHNF

SEQ ID NO: 43 - protein, 34 aa, Homo sapiens
AVSEIQLMHNLGKHLNSMERVEWLRKKLQDVHNF

SEQ ID NO: 44 - protein, 36 aa, Homo sapiens; identical to SEQ ID NO: 10
  SITE 1: note = MISC_FEATURE - Xaa at position 1 is a dLys having its side chain acylated with COC16H32CO2H) via a gamma Glu spacer

SEQ ID NO: 45 - protein, 36 aa, Homo sapiens; identical to SEQ ID NO: 11
  SITE 1: note = MISC_FEATURE - Xaa at position 1 is a dLys having its side chain acylated with COC16H32CO2H via a gamma Glu spacer
  SITE 2: note = MISC_FEATURE - Xaa at position 2 is Sarcosine (n-methyl Glycine)

SEQ ID NO: 46 - protein, 36 aa, Homo sapiens; identical to SEQ ID NO: 11
  SITE 1: note = MISC_FEATURE - Xaa at position 1 is a dLys having its side chain acylated with COC16H32CO2H via a gamma Glu spacer
  SITE 2: note = MISC_FEATURE - Xaa at position 2 is n-isopropyl-Glycine

SEQ ID NO: 47 - protein, 36 aa, Homo sapiens; identical to SEQ ID NO: 10
  SITE 1: note = MISC_FEATURE - Xaa at position 1 is dLys

SEQ ID NO: 48 - protein, 36 aa, Homo sapiens; identical to SEQ ID NO: 11
  SITE 1: note = MISC_FEATURE - Xaa at position 1 is a Lys having its side chain acylated with COC16H32CO2H) via a gamma Glu spacer
  SITE 2: note = MISC_FEATURE - Xaa at position 2 is n-isopropyl-Glycine

SEQ ID NO: 49 - protein, 36 aa, Homo sapiens
  SITE 1: note = MISC_FEATURE - Xaa at position 1 is a dLys having its side chain acylated with COC16H32CO2H) via a gamma Glu spacer
  SITE 2: note = MISC_FEATURE - Xaa at position 2 is n-isopropyl-Glycine
  SITE 3: note = MISC_FEATURE - Xaa at position 3 is dSer
XXXVSEIQLMHNLGKHLNSMERVEWLRKKLQDVHNF

SEQ ID NO: 50 - protein, 36 aa, Homo sapiens
  SITE 1: note = MISC_FEATURE - Xaa at position 1 is a dLys having its side chain acylated with COC16H32CO2H) via a gamma Glu spacer
XPSVSEIQLMHNLGKHLNSMERVEWLRKKLQDVHNF

SEQ ID NO: 51 - protein, 36 aa, Homo sapiens; identical to SEQ ID NO: 50
  SITE 1: note = MISC_FEATURE - Xaa at position 1 is a Lys having its side chain acylated with COC16H32CO2H) via a gamma Glu spacer

SEQ ID NO: 52 - protein, 36 aa, Homo sapiens; identical to SEQ ID NO: 11
  SITE 1: note = MISC_FEATURE - Xaa at position 1 is a Lys having its side chain acylated with COC16H32CO2H) via a gamma Glu spacer
  SITE 2: note = MISC_FEATURE - Xaa at position 2 is Sarcosine (n-methyl Glycine)

SEQ ID NO: 53 - protein, 36 aa, Homo sapiens; identical to SEQ ID NO: 11
  SITE 1: note = MISC_FEATURE - Xaa at position 1 is a Lys having its side chain acylated with COC16H32CO2H) via a gamma Glu spacer
  SITE 2: note = MISC_FEATURE - Xaa at position 2 is n-methyl-Alanine

SEQ ID NO: 54 - protein, 36 aa, Homo sapiens; identical to SEQ ID NO: 11
  SITE 1: note = MISC_FEATURE - Xaa at position 1 is a Lys having its side chain acylated with COC16H32CO2H) via a gamma Glu spacer
  SITE 2: note = MISC_FEATURE - Xaa at position 2 is n-methyl-Alanine
  SITE 2: note = MISC_FEATURE - Xaa at position 2 is Pipecolic acid (piperidine-2-carboxylic acid)

SEQ ID NO: 55 - protein, 34 aa, Homo sapiens
SVSEIQLMHNLGKHLNSMERVEWLRKKLQDVHQF

SEQ ID NO: 56 - protein, 35 aa, Homo sapiens; identical to SEQ ID NO: 12
  SITE 35: note = MISC_FEATURE - Xaa at position 35 is Lys that has an amide substituted for the native carboxyl group wherein the side chain is acylated with COC16H32CO2H via a gamma Glu spacer

SEQ ID NO: 57 - protein, 37 aa, Homo sapiens; identical to SEQ ID NO: 13
  SITE 1: note = MISC_FEATURE - Xaa at position 1 is dLys
  SITE 2: note = MISC_FEATURE - Xaa at position 2 is Sarcosine (n-methyl-Glysine)
  SITE 37: note = MISC_FEATURE - Xaa at position 37 is Lys that has an amide substituted for the native carboxyl group wherein the side chain is acylated with COC16H32CO2H via a gamma Glu spacer

SEQ ID NO: 58 - protein, 37 aa, Homo sapiens; identical to SEQ ID NO: 13
  SITE 1: note = MISC_FEATURE - Xaa at position 1 is dLys wherein the side chain is acylated with COC16H32CO2H via a gamma Glu spacer
  SITE 2: note = MISC_FEATURE - Xaa at position 2 is Sarcosine (n-methyl-Glysine)
  SITE 37: note = MISC_FEATURE - Xaa at position 37 is Lys that has an amide substituted for the native carboxyl group wherein th eside chain is acylated with COC16H32CO2H via a gamma Glu spacer

SEQ ID NO: 59 - protein, 33 aa, Homo sapiens; identical to SEQ ID NO: 31

SEQ ID NO: 60 - protein, 33 aa, Homo sapiens; identical to SEQ ID NO: 2
  SITE 33: note = MISC_FEATURE - Xaa at position 33 is aspartic acid wherein the carboxy terminus is replace with an amide

SEQ ID NO: 61 - protein, 36 aa, Homo sapiens; identical to SEQ ID NO: 14
  SITE 1: note = MISC_FEATURE - Xaa at position 1 is dLys wherein the side chain is acylated with COC16H32CO2H via a gamma Glu spacer
  SITE 2: note = MISC_FEATURE - Xaa at position 2 is Sarcosine (n-methyl-Glysine)

SEQ ID NO: 62 - protein, 29 aa, Homo sapiens
QLMHNLGKHLNSMERVEWLRKKLQDGHNF

SEQ ID NO: 63 - protein, 38 aa, Homo sapiens; identical to SEQ ID NO: 21
  SITE 38: note = MISC_FEATURE - Xaa at position 38 is Lys that has an amide substituted for the native carboxyl group wherein the side chain is acylated with COC16H32CO2H via a gamma Glu spacer

SEQ ID NO: 64 - protein, 33 aa, Homo sapiens
  SITE 33: note = MISC_FEATURE - Xaa at position 33 is Lys wherein the side chain is acylated with COC16H32CO2H via a gamma Glu spacer
SVSEIQLMHNLGRHLNSMERVEWLRRRLQDVHX

SEQ ID NO: 65 - protein, 37 aa, Homo sapiens
  SITE 1: note = MISC_FEATURE - Xaa at position 1 is dLys wherein the side chain is acylated with COC16H32CO2H via a gamma Glu-(MINIPEG)2-gamma Glu spacer
  SITE 37: note = MISC_FEATURE - Xaa at position 37 is Lys that has an amide substituted for the native carboxyl group wherein the side chain is acylated with COC16H32CO2H via a gamma Glu-(MINIPEG)2-gamma Glu spacer
XGSVSEIQLMHNLGKHLNSMERVEWLRKKLQDVHNFX

SEQ ID NO: 66 - protein, 35 aa, Homo sapiens
  SITE 35: note = MISC_FEATURE - Xaa at position 35 is Lys that has an amide substituted for the native carboxyl group wherein the side chain is acylated with COC16H32CO2H via a gamma Glu spacer
SVSEIQLMHNLGKHLNSMERVEWLRKELQDVHNFX

SEQ ID NO: 67 - protein, 35 aa, Homo sapiens
  SITE 35: note = MISC_FEATURE - Xaa at position 35 is Lys that has an amide substituted for the native carboxyl group wherein the side chain is acylated with COC16H32CO2H via a gamma Glu spacer
SVSEIQLMHNLGKHLNSMERVEWLREKLQDVHNFX

SEQ ID NO: 68 - protein, 35 aa, Homo sapiens
  SITE 35: note = MISC_FEATURE - Xaa at position 35 is Lys that has an amide substituted for the native carboxyl group wherein the side chain is acylated with COC16H32CO2H via a gamma Glu spacer
SVSEIQLMHNLGEHLNSMERVEWLRKKLQDVHNFX

SEQ ID NO: 69 - protein, 35 aa, Homo sapiens
  SITE 20: note = MISC_FEATURE - Xaa at position 20 is Aib
  SITE 35: note = MISC_FEATURE - Xaa at position 35 is Lys that has an amide substituted for the native carboxyl group wherein the side chain is acylated with COC16H32CO2H via a gamma Glu spacer
SVSEIQLMHNLGKHLNSMEXVEWLRKKLQDVHNFX

SEQ ID NO: 70 - protein, 35 aa, Homo sapiens
  SITE 17: note = MISC_FEATURE - Xaa at position 17 is Aib
  SITE 35: note = MISC_FEATURE - Xaa at position 35 is Lys that has an amide substituted for the native carboxyl group wherein the side chain is acylated with COC16H32CO2H via a gamma Glu spacer
SVSEIQLMHNLGKHLNXMERVEWLRHKLQDVHNFX

SEQ ID NO: 71 - protein, 35 aa, Homo sapiens
  SITE 16: note = MISC_FEATURE - Xaa at position 16 is Aib
  SITE 35: note = MISC_FEATURE - Xaa at position 35 is Lys that has an amide substituted for the native carboxyl group wherein the side chain is acylated with COC16H32CO2H via a gamma Glu spacer
SVSEIQLMHNLGKHLXSMERVEWLRKKLQDVHNFX

SEQ ID NO: 72 - protein, 35 aa, Homo sapiens
  SITE 12: note = MISC_FEATURE - Xaa at position 12 is Aib
  SITE 35: note = MISC_FEATURE - Xaa at position 35 is Lys that has an amide substituted for the native carboxyl group wherein the side chain is acylated with COC16H32CO2H via a gamma Glu spacer
SVSEIQLMHNLXKHLNSMERVEWLRKKLQDVHNFX

SEQ ID NO: 73 - protein, 34 aa, Homo sapiens
  SITE 33: note = MISC_FEATURE - Xaa at position 33 is Lys wherein the side chain is acylated with COC16H32CO2H via a gamma Glu spacer
SVSEIQLMHNLGEHLNSMERVEWLREKLQDVHXF

SEQ ID NO: 74 - protein, 33 aa, Homo sapiens
  SITE 33: note = MISC_FEATURE - Xaa at position 33 is Lys wherein the side chain is acylated with COC16H32CO2H via a gamma Glu spacer
SVSEIQLMHNLGEHLNSMERVEWLREKLQDVHX

SEQ ID NO: 75 - protein, 33 aa, Homo sapiens
  SITE 26: note = MISC_FEATURE - Xaa at position 33 is Lys wherein the side chain is acylated with COC16H32CO2H via a gamma Glu spacer
SVSEIQLMHNLGKHLNSMERVEWLRXKLQDVHQ

SEQ ID NO: 76 - protein, 33 aa, Homo sapiens; identical to SEQ ID NO: 74
  SITE 33: note = MISC_FEATURE - Xaa at position 33 is Lys wherein the side chain is acylated with COC16H32CO2H via a (gamma Glu)3x spacer

SEQ ID NO: 77 - protein, 33 aa, Homo sapiens; identical to SEQ ID NO: 2
  SITE 33: note = MISC_FEATURE - Xaa at position 35 is Lys wherein the side 3hain is acylated with COC16H32CO2H via a gamma Glu-(MINIPEG)2-gamma Glu spacer

SEQ ID NO: 78 - protein, 35 aa, Homo sapiens; identical to SEQ ID NO: 19
  SITE 1: note = MISC_FEATURE - Xaa at position 1 is dLys
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 79 - protein, 35 aa, Homo sapiens; identical to SEQ ID NO: 19
  SITE 1: note = MISC_FEATURE - Xaa at position 1 is dLys wherein the side chain is acylated with COC16H32CO2H via a gamma Glu-(MINIPEG)2-gamma Glu spacer
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 80 - protein, 35 aa, Homo sapiens; identical to SEQ ID NO: 17
  SITE 1: note = MISC_FEATURE - Xaa at position 1 is dLys
  SITE 2: note = MISC_FEATURE - Xaa at position2 is Sarcosine (n-methyl-Glycine)
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 81 - protein, 35 aa, Homo sapiens; identical to SEQ ID NO: 17
  SITE 1: note = MISC_FEATURE - Xaa at position 1 is dLys
  SITE 2: note = MISC_FEATURE - Xaa at position2 is n-isopropylGlycine
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 82 - protein, 35 aa, Homo sapiens; identical to SEQ ID NO: 17
  SITE 1: note = MISC_FEATURE - Xaa at position 1 is Aib
  SITE 2: note = MISC_FEATURE - Xaa at position2 is n-imethyl-Glycine
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 83 - protein, 33 aa, Homo sapiens; identical to SEQ ID NO: 2
  SITE 33: note = MISC_FEATURE - Xaa at position 33 is Lys wherein the side 3hain is acylated with COC16H32CO2H via a lysine-gamma Glu-(MINIPEG)2-gamma Glu spacer

SEQ ID NO: 84 - protein, 35 aa, Homo sapiens; identical to SEQ ID NO: 19
  SITE 1: note = MISC_FEATURE - Xaa at position 1 is dLys
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 85 - protein, 35 aa, Homo sapiens; identical to SEQ ID NO: 17
  SITE 1: note = MISC_FEATURE - Xaa at position 1 is dLys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COCH3

SEQ ID NO: 86 - protein, 35 aa, Homo sapiens; identical to SEQ ID NO: 17
  SITE 1: note = MISC_FEATURE - Xaa at position 1 is dLys wherein the side chain is acylated with COC15CH31 via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 87 - protein, 35 aa, Homo sapiens; identical to SEQ ID NO: 17
  SITE 1: note = MISC_FEATURE - Xaa at position 1 is dLys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 88 - protein, 33 aa, Homo sapiens
SVSEIQAMHNLGKHLNSMERVEWLRKKLQDVHK

SEQ ID NO: 89 - protein, 33 aa, Homo sapiens
SVSEIQLAHNLGKHLNSMERVEWLRKKLQDVHK

SEQ ID NO: 90 - protein, 33 aa, Homo sapiens
SVSEIQLMHNLGKHLNSMEAVEWLRKKLQDVHN

SEQ ID NO: 91 - protein, 33 aa, Homo sapiens
SVSEIQLMHNLGKHLNSMERVEALRKKLQDVHK

SEQ ID NO: 92 - protein, 33 aa, Homo sapiens
SVSEIQLMHNLGKHLNSMERVEWARKKLQDVHK

SEQ ID NO: 93 - protein, 33 aa, Homo sapiens; identical to SEQ ID NO: 18
  SITE 33: note = MISC_FEATURE - Xaa at position 33 is Lys wherein the side chain is acylated with COC16H32CO2H via a gamma Glu-(MINIPEG)2-gamma Glu spacer

SEQ ID NO: 94 - protein, 35 aa, Homo sapiens
  SITE 1: note = MISC_FEATURE - Xaa at position 1 is dLys
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIQLAHNLGKHLNSMERVEWLRKKLQDVHX

SEQ ID NO: 95 - protein, 35 aa, Homo sapiens; identical to SEQ ID NO: 94
  SITE 1: note = MISC_FEATURE - Xaa at position 1 is dLys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 96 - protein, 38 aa, Homo sapiens
  SITE 38: note = MISC_FEATURE - Xaa at position 38 is an acylated amino acid
VSEHQLLHDKGKSIQDLRRRFFLHHLIAEIHTAEIRAX

SEQ ID NO: 97 - protein, 32 aa, Homo sapiens
SEIQLMHNLGKHLNSMERVEWLRKKLQDVHNF

SEQ ID NO: 98 - protein, 34 aa, Homo sapiens; identical to SEQ ID NO: 9

SEQ ID NO: 99 - protein, 29 aa, Homo sapiens
QLMHNLGKHLNSMERVEWLREKLQDVHNF

SEQ ID NO: 100 - protein, 34 aa, Homo sapiens
SVSEIQLMHNLGKHLNSMERVEWLREKLQDVHNF

SEQ ID NO: 101 - protein, 34 aa, Homo sapiens; identical to SEQ ID NO: 100

SEQ ID NO: 102 - protein, 34 aa, Homo sapiens; identical to SEQ ID NO: 8
  SITE 33: note = MISC_FEATURE - Xaa at position 33 is Lys wherein the side chain is acylated with COC16H32CO2H via a gamma Glu spacer

SEQ ID NO: 103 - protein, 33 aa, Homo sapiens
  VARIANT 12: note = MISC_FEATURE - Xaa at position 12 is amino isobutyric acid (Aib) or Gly
  VARIANT 13: note = MISC_FEATURE - Xaa at position 13 is Arg, Glu, Asp or Lys
  VARIANT 16: note = MISC_FEATURE - Xaa at position 16 is amino isobutyric acid (Aib) or Asn
  VARIANT 17: note = Xaa at position 16 is amino isobutyric acid (Aib) or Ser
  VARIANT 26: note = MISC_FEATURE - Xaa at position 26 is Arg, Glu, Asp or Lys
  VARIANT 27: note = MISC_FEATURE - Xaa at position 27 is Arg, Glu, Asp or Lys
  VARIANT 31: note = MISC_FEATURE - Xaa at position 31 is Gly or Val
SVSEIQLMHNLXXHLXXMERVEWLRXXLQDXHN

SEQ ID NO: 104 - protein, 33 aa, Homo sapiens
  VARIANT 13: note = MISC_FEATURE - Xaa at position 13 is Arg, Glu, Asp or Lys
  VARIANT 26: note = MISC_FEATURE - Xaa at position 26 is Arg, Glu, Asp or Lys
  VARIANT 27: note = MISC_FEATURE - Xaa at position 27 is Arg, Glu, Asp or Lys
  VARIANT 31: note = MISC_FEATURE - Xaa at position 31 is is Gly or Val
SVSEIQLMHNLGXHLNSMERVEWLRXXLQDXHN

SEQ ID NO: 105 - protein, 33 aa, Homo sapiens
  VARIANT 12: note = MISC_FEATURE - Xaa a position 16 is amino isobutyric acid (Aib) or Gly
  VARIANT 16: note = MISC_FEATURE - Xaa a position 16 is amino isobutyric acid (Aib) or Asn
  VARIANT 17: note = MISC_FEATURE - Xaa a position 17 is amino isobutyric acid (Aib) or Ser
SVSEIQLMHNLXKHLXXMERVEWLRKKLQDVHN

SEQ ID NO: 106 - protein, 33 aa, Homo sapiens; identical to SEQ ID NO: 31

SEQ ID NO: 107 - protein, 35 aa, Homo sapiens; identical to SEQ ID NO: 94
  SITE 1: note = MISC_FEATURE - Xaa at position 1 is Lys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 108 - protein, 35 aa, Homo sapiens; identical to SEQ ID NO: 17
  SITE 1: note = MISC_FEATURE - Xaa at position 1 is Lys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer

SEQ ID NO: 109 - protein, 33 aa, Homo sapiens
  SITE 8: note = MISC_FEATURE - Xaa at position 8 is Met wherein the side chain is oxidized to form a sulfoxide
  SITE 33: note = MISC_FEATURE - Xaa at position 33 is Lys wherein the side chain is acylated with COC16H32CO2H via a gamma Glu-(MINIPEG)2-gamma Glu spacer
SVSEIQLXHNLGKHLNSMERVEWLRKKLQDVHX

SEQ ID NO: 110 - protein, 33 aa, Homo sapiens
  SITE 18: note = MISC_FEATURE - Xaa at position 18 is Met wherein the side chain is oxidized to form a sulfoxide
  SITE 33: note = MISC_FEATURE - Xaa at position 33 is Lys wherein the side chain is acylated with COC16H32CO2H via a gamma Glu-(MINIPEG)2-gamma Glu spacer
SVSEIQLMHNLGKHLNSXERVEWLRKKLQDVHX

SEQ ID NO: 111 - protein, 35 aa, Homo sapiens; identical to SEQ ID NO: 17
  SITE 1: note = MISC_FEATURE - Xaa at position 1 is dLys wherein the side chain is acylated with COC16H32CO2H via a Tyr(OPO3H2)-(miniPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(miniPEG)2-gamma Glu spacer

SEQ ID NO: 112 - protein, 35 aa, Homo sapiens; identical to SEQ ID NO: 17
  SITE 1: note = MISC_FEATURE - Xaa at position 1 is dLys wherein the side chain is acylated with COC16H32CO2H via a homoCysr(SO3H)-(miniPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(miniPEG)2-gamma Glu spacer

SEQ ID NO: 113 - protein, 35 aa, Homo sapiens; identical to SEQ ID NO: 17
  SITE 1: note = MISC_FEATURE - Xaa at position 1 is dLys wherein the side chain is acylated with COC16H32CO2H via a Arg-(miniPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(miniPEG)2-gamma Glu spacer

SEQ ID NO: 114 - protein, 35 aa, Homo sapiens; identical to SEQ ID NO: 17
  SITE 1: note = MISC_FEATURE - Xaa at position 1 is dLys wherein the side chain is acylated with COC16H32CO2H via a Arg-(miniPEG)2-Tyr(OPO3H2)-(miniPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(miniPEG)2-gamma Glu spacer

SEQ ID NO: 115 - protein, 35 aa, Homo sapiens; identical to SEQ ID NO: 17
  SITE 1: note = MISC_FEATURE - Xaa at position 1 is dLys wherein the side chain is acylated with COC16H32CO2H via a Tyr(OPO3H2)-(miniPEG)2-Tyr(OPO3H2)-(miniPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(miniPEG)2-gamma Glu spacer

SEQ ID NO: 116 - protein, 35 aa, Homo sapiens; identical to SEQ ID NO: 17
  SITE 1: note = MISC_FEATURE - Xaa at position 1 is Lys wherein the side chain is acylated with COC16H32CO2H via a Tyr(OPO3H2)-(miniPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a Tyr(OPO3H2)-gamma Glu-(miniPEG)2-gamma Glu spacer

SEQ ID NO: 117 - protein, 35 aa, Homo sapiens
  SITE 1: note = MISC_FEATURE - Xaa at position 1 is Lys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIELMHNLGKHLNSMERVEWLRKKLQDVHX

SEQ ID NO: 118 - protein, 33 aa, Homo sapiens
  SITE 33: note = MISC_FEATURE - Xaa at position 33 is Lys wherein the side chain is acylated with COC16H32CO2H via a gamma Glu-(MINIPEG)2-gamma Glu spacer
SVSEIELMHNLGKHLNSMERVEWLRKKLQDVHX

SEQ ID NO: 119 - protein, 35 aa, Homo sapiens
  SITE 1: note = MISC_FEATURE - Xaa at position 1 is Lys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIQLMHDLGKHLNSMERVEWLRKKLQDVHX

SEQ ID NO: 120 - protein, 33 aa, Homo sapiens
  SITE 33: note = MISC_FEATURE - Xaa at position 33 is Lys wherein the side chain is acylated with COC16H32CO2H via a gamma Glu-(MINIPEG)2-gamma Glu spacer
SVSEIQLMHDLGKHLNSMERVEWLRKKLQDVHX

SEQ ID NO: 121 - protein, 35 aa, Homo sapiens
  SITE 1: note = MISC_FEATURE - Xaa at position 1 is Lys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIQLMHNLGKHLDSMERVEWLRKKLQDVHX

SEQ ID NO: 122 - protein, 33 aa, Homo sapiens
  SITE 33: note = MISC_FEATURE - Xaa at position 33 is Lys wherein the side chain is acylated with COC16H32CO2H via a gamma Glu-(MINIPEG)2-gamma Glu spacer
SVSEIQLMHNLGKHLDSMERVEWLRKKLQDVHX

SEQ ID NO: 123 - protein, 35 aa, Homo sapiens
  SITE 1: note = MISC_FEATURE - Xaa at position 1 is Lys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIQLMHNLGKHLNSMERVEWLRKKLEDVHX

SEQ ID NO: 124 - protein, 33 aa, Homo sapiens
  SITE 33: note = MISC_FEATURE - Xaa at position 33 is Lys wherein the side chain is acylated with COC16H32CO2H via a gamma Glu-(MINIPEG)2-gamma Glu spacer
SVSEIQLMHNLGKHLNSMERVEWLRKKLEDVHX

SEQ ID NO: 125 - protein, 35 aa, Homo sapiens
  SITE 1: note = MISC_FEATURE - Xaa at position 1 is dLys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 18: note = MISC_FEATURE - Xaa at position 18 is iso-aspartic acid
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIQLMHNLGKHLXSMERVEWLRKKLQDVHX

SEQ ID NO: 126 - protein, 35 aa, Homo sapiens
  SITE 1: note = MISC_FEATURE - Xaa at position 1 is dLys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 12: note = MISC_FEATURE - Xaa at position 12 is iso-aspartic acid
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIQLMHXLGKHLNSMERVEWLRKKLQDVHX

SEQ ID NO: 127 - protein, 35 aa, Homo sapiens
  SITE 1: note = MISC_FEATURE - Xaa at position 1 is dLys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 10: note = MISC_FEATURE - Xaa at position 10 is Met wherein the side chain is oxidized to form a sulfoxide
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIQLXHNLGKHLNSMERVEWLRKKLQDVHX

SEQ ID NO: 128 - protein, 35 aa, Homo sapiens
  SITE 1: note = MISC_FEATURE - Xaa at position 1 is dLys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 20: note = MISC_FEATURE - Xaa at position 20 is Met wherein the side chain is oxidized to form a sulfoxide
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIQLMHNLGKHLNSXERVEWLRKKLQDVHX

SEQ ID NO: 129 - protein, 34 aa, Homo sapiens
SVSEIQLMHALGKHLNSMERVEWLRKKLQDVHNF

SEQ ID NO: 130 - protein, 34 aa, Homo sapiens
SVSEIQLMHFLGKHLNSMERVEWLRKKLQDVHNF

SEQ ID NO: 131 - protein, 34 aa, Homo sapiens
SVSEIQLMHWLGKHLNSMERVEWLRKKLQDVHNF

SEQ ID NO: 132 - protein, 35 aa, Homo sapiens
  SITE 1: note = MISC_FEATURE - Xaa at position 1 is dLys wherein the side chain is acylated with COC16H32CO2H via a gamma Glu-(MINIPEG)2-gamma Glu spacer
  SITE 2: note = MISC_FEATURE - Xaa at position 2 is n-methyl glycine
  SITE 20: note = MISC_FEATURE - Xaa at position 20 is Met wherein the side chain is oxidized to form a sulfoxide
  SITE 35: note = MISC_FEATURE - Xaa at position 35 is Lys wherein the side chain is acylated with COC16H32CO2H via a gamma Glu-(MINIPEG)2-gamma Glu spacer
XXSVSEIQLMHNLGKHLNSMERVVWLRKKLQDVHX

SEQ ID NO: 133 - protein, 32 aa, Homo sapiens
  VARIANT 10: note = MISC_FEATURE - Xaa at position 10 is Asp, Gln or Asn
  VARIANT 13: note = MISC_FEATURE - Xaa at position 13 is Arg, Glu, Asp or Lys
  VARIANT 16: note = MISC_FEATURE - Xaa at position 16 is Asp, Gln or Asn
  VARIANT 18: note = MISC_FEATURE - Xaa at position 18 is Met, Met(O), Leu, or Nleu
  VARIANT 26: note = MISC_FEATURE - Xaa at position 26 is Arg, Glu, Asp or Lys
  VARIANT 27: note = MISC_FEATURE - Xaa at position 27 is Arg, Glu, Asp or Lys
  VARIANT 31: note = MISC_FEATURE - Xaa at position 31 is Gly or Val
SVSEIQLMHXLGXHLXSXERVEWLRXXLQDXH

SEQ ID NO: 134 - protein, 32 aa, Homo sapiens
  VARIANT 10: note = MISC_FEATURE - Xaa at position 10 is Asp, Gln or Asn
  VARIANT 12: note = MISC_FEATURE - Xaa at position 12 is isobutyric acid (Aib) or Gly
  VARIANT 16: note = MISC_FEATURE - Xaa at position 16 is isobutyric acid (Aib) or Asn
  VARIANT 17: note = MISC_FEATURE - Xaa at position 17 is amino isobutyric acid (Aib) or Ser
  VARIANT 18: note = MISC_FEATURE - Xaa at position 18 is Met, Met(O), Leu, or Nleu
SVSEIQLMHXLXKHLXXXERVEWLRKKLQDVH

SEQ ID NO: 135 - protein, 32 aa, Homo sapiens
  VARIANT 10: note = MISC_FEATURE - Xaa at position 10 is Asp, Gln or Asn
  VARIANT 16: note = MISC_FEATURE - Xaa at position 16 is Asp, Gln or Asn
  VARIANT 18: note = MISC_FEATURE - Xaa at position 18 is Met, Met(O), Leu, or Nleu
SVSEIQLMHXLGKHLXSXERVEWLRKKLQDVH

SEQ ID NO: 136-137 - skipped (2 entries)

SEQ ID NO: 138 - protein, 6 aa, synthetic construct
  REGION 1..6: note = synthetic aminoacid extension
  VARIANT 1: note = MISC_FEATURE - Xaa at position 1 is Lys, dLys, ornithine, Cys or homocysteine acylated with a C16-C30 fatty acid or C16-C30 diacid
  VARIANT 6: note = MISC_FEATURE - Xaa at position 6 is Lys, dLys, ornithine, Cys or homocysteine acylated with a C16-C30 fatty acid or C16-C30 diacid
XFVALX

SEQ ID NO: 139 - protein, 7 aa, synthetic construct
  REGION 1..7: note = synthetic aminoacid extension
  VARIANT 1: note = MISC_FEATURE - Xaa at position 1 is Lys, dLys, ornithine, Cys or homocysteine acylated with a C16-C30 fatty acid or C16-C30 diacid
  VARIANT 7: note = MISC_FEATURE - Xaa at position 7 is Lys, dLys, ornithine, Cys or homocysteine acylated with a C16-C30 fatty acid or C16-C30 diacid
XFVALGX

SEQ ID NO: 140-141 - skipped (2 entries)

SEQ ID NO: 142 - protein, 6 aa, synthetic construct
  REGION 1..6: note = synthetic aminoacid extension
  VARIANT 1: note = MISC_FEATURE - Xaa at position 1 is Lys, dLys, ornithine, Cys or homocysteine acylated with a C16-C30 fatty acid or C16-C30 diacid
  VARIANT 6: note = MISC_FEATURE - Xaa at position 6 is Gln or Asn
XFVALX

SEQ ID NO: 143 - protein, 7 aa, synthetic construct
  REGION 1..7: note = synthetic aminoacid extension
  VARIANT 1: note = MISC_FEATURE - Xaa at position 1 is Lys, dLys, ornithine, Cys or homocysteine acylated with a C16-C30 fatty acid or C16-C30 diacid
  VARIANT 7: note = MISC_FEATURE - Xaa at position 7 is Gln or Asn
XFVALGX

SEQ ID NO: 144 - protein, 4 aa, Homo sapiens
FVAL

SEQ ID NO: 145 - protein, 5 aa, Homo sapiens
FVALG

SEQ ID NO: 146 - protein, 6 aa, Homo sapiens
FVALGA

SEQ ID NO: 147 - protein, 4 aa, Homo sapiens
FVAL

SEQ ID NO: 148 - protein, 5 aa, Homo sapiens
FVALG

SEQ ID NO: 149 - protein, 6 aa, Homo sapiens
FVALGA